(19) United States
(12) Patent Application Publication
(10) Pub. No.: US 2023/0219282 A1
(43) Pub. Date: Jul. 13, 2023

(54) ULTRASOUND RESPONSIVE SHAPE MEMORY POLYMER COMPOSITES

(71) Applicant: California Institute of Technology, Pasadena, CA (US)

(72) Inventors: Max KUDISCH, Pasadena, CA (US); Jeong Hoon KO, Pasadena, CA (US); Chiara DARAIO, Pasadena, CA (US); Mikhail G. SHAPIRO, Pasadena, CA (US); Di WU, Pasadena, CA (US); Gunho KIM, Pasadena, CA (US); Robert H. GRUBBS, South Pasadena, CA (US); Ki-Young YOON, Bridgewater, NJ (US)

(21) Appl. No.: 18/096,251

(22) Filed: Jan. 12, 2023

Related U.S. Application Data
(60) Provisional application No. 63/299,256, filed on Jan. 13, 2022.

(51) Int. Cl. B29C 61/06; A61L 31/14; A61L 31/12

(52) U.S. Cl. CPC B29C 61/06; A61L 31/14; A61L 31/128; A61L 2400/16; B29K 2995/0012; B29K 2509/02

(57) ABSTRACT

Aspects disclosed herein include a composite material comprising: one or more shape memory polymers; and a first additive provided in the shape memory polymer(s); wherein: the first additive increases one or more ultrasound-absorption characteristics of the composite material compared to that of the same shape memory polymer(s) free of said first additive; the composite material is characterized by a composite transition temperature (T_{cm,trans}); and the composite material or one or more portions thereof undergo a shape change from a temporary shape to a permanent shape when the composite material or said one or more portions thereof are heated to within 35° C. of T_{cm,trans} or a temperature approximately equal to or greater than T_{cm,trans}.

### CROSS-REFERENCE TO RELATED APPLICATIONS

This application claims the benefit of priority to U.S. Provisional Patent Application No. 63/299,256, filed Jan. 13, 2022, which is hereby incorporated by reference in its entirety.

### BACKGROUND OF INVENTION

Shape memory polymers (SMPs) are polymers which demonstrate shape memory behavior characterized by a spontaneous change in shape triggered by an external stimulus. These polymers possess a permanent shape, corresponding to the shape at formation of chemical or physical crosslinks, and a temporary shape, which may correspond to the shape at crystallization of ordered domains and/or vitrification, for example. The SMP can spontaneously return from its temporary shape to its permanent shape when approaching, at, or exceeding the characteristic transition temperature, Tₜᵣₐₙₛ.

The shape memory process can be used to perform a desired function as a component of a medical device or tool. For example, shape memory polymers have been used for drug delivery, to make self-tightening sutures, for auto-expanding stents, and for other biomedical applications. However, most existing applications make use of heat as the stimulus to trigger a shape memory effect, with relatively few examples of other stimuli.

Significant challenges exist in the art with respect to utilizing shape memory polymer for medical devices. A significant challenge is the external stimulus, the type and characteristics of which are consequential to the usefulness or effectiveness of an SMP or a device with an SMP. For example, while heat is a typical stimulus for triggering the shape memory effect (or, shape change), it is unspecific or non-localized and may easily cause damage to neighboring tissues. Light as a stimulus may be problematic as the relevant wavelength may not sufficiently penetrate skin. Chemical exposure as a stimulus carries the risk of toxicity or other chemical-induced negative effects. Other challenges in the art include fine control over the Tₜᵣₐₙₛ to precisely tailor an SMP to a particular medical application, stability under physiological conditions, and minimizing biological toxicity. These, and other, challenges are interdependent. For example, an SMP that has a useful Tₜᵣₐₙₛ may have stability and/or toxicity issues or may practically only be triggered by a stimulus that is harmful or otherwise counterproductive. These and other challenges are addressed by materials, methods, and devices disclosed herein.

### SUMMARY OF THE INVENTION

Provided herein are materials, methods, devices, and associated aspects, that provide advantages of the shape memory effect of shape memory polymers while obviating disadvantages and addressing challenges such as those discussed above. In various aspects disclosed herein, composite materials disclosed herein provide the shape memory effect advantage of SMPs and further provide for fine tunability of Tₜᵣₐₙₛ, remote non-invasive and biologically-benign triggering using focused ultrasound, ease and high degree of malleability, stability under physiological or in-vivo conditions, and precise control over the degree and location of shape change. These features allow the composite materials disclosed herein to be adapted to a wide range of medical devices, as well as to devices for non-medical applications.

Aspects disclosed herein include a composite material comprising: one or more shape memory polymers; and a first additive provided in the shape memory polymer(s); wherein: the first additive comprises or is (optionally, is) a plurality of inorganic particles; the composite material is characterized by a composite transition temperature (T_{cm,trans}); and the composite material or one or more portions thereof undergo a shape change from a temporary shape to a permanent shape when the composite material or said one or more portions thereof are heated to or beyond T_{cm,trans}.

Aspects disclosed herein include a composite material comprising: one or more shape memory polymers; and a first additive provided in the shape memory polymer(s); wherein: the first additive increases one or more ultrasound-attenuation characteristics of the composite material compared to that of the shape memory polymer(s) alone; the composite material is characterized by a composite transition temperature (T_{cm,trans}); and the composite material or one or more portions thereof undergo a shape change from a temporary shape to a permanent shape when the composite material or said one or more portions thereof are heated to within 35° C. (optionally within 30° C., optionally within 25° C., optionally within 20° C., optionally within 15° C., optionally within 10° C., optionally within 5° C.) of T_{cm,trans} or a temperature approximately equal to (e.g., “approximately equal”=within 20%, optionally within 10%, optionally within 5%, optionally within 1%, optionally equal) or greater than T_{cm,trans}.

Aspects disclosed herein include a composite material comprising: one or more shape memory polymers; a first additive provided in the shape memory polymer(s); wherein: the first additive comprises or is (optionally, is) a plurality of hollow particles; the composite material is characterized by a composite transition temperature (T_{cm,trans}); and the composite material or one or more portions thereof undergo a shape change from a temporary shape to a permanent shape when the composite material or said one or more portions thereof are heated to or beyond T_{cm,trans}.

Aspects disclosed herein include a method of using a composite material, the method comprising: directing one or more focused ultrasound beams at one or more portions of the composite material; thereby, heating the one or more portions to a temperature approximately equal to or greater than a composite transition temperature (T_{cm,trans}); and thereby, causing the composite material to undergo a shape change at the one or more portions thereof; wherein the composite material comprises: one or more shape memory polymers; and a first additive provided in the shape memory polymer(s); wherein: the first additive (a) comprises or is (optionally, is) a plurality of inorganic particles, (b) increases an ultrasound-attenuation characteristic of the composite material compared to that of the shape memory polymer(s) alone, and/or (c) comprises or is (optionally, is) a plurality of hollow particles; the composite material is characterized by the composite transition temperature (T_{cm,trans}); and the one or more portions of the composite material undergo the shape change from a temporary shape to a permanent shape when the one or more portions are heated to within 35° C. (optionally within 30° C., optionally within 25° C., optionally within 20° C., optionally within 15° C., optionally within 10° C., optionally within 5° C.) of T_{cm,trans} or a temperature approximately equal to (e.g., “approximately equal”=within 20%, optionally within 10%, optionally within 5%, optionally within 1%, optionally equal) or greater than T_{cm,trans}.

Aspects disclosed herein include a method of making a composite material, the method comprising: polymerizing a monomer to form a first polymer; crosslinking the first polymer in the presence of a crosslinking precursor and the first additive at a temperature equal to or greater than a crosslinking temperature (T_{cm,crosslink}) to form the composite material having the crosslinked shape memory polymer and the first additive; wherein the composite material comprises: one or more shape memory polymers; and a first additive provided in the shape memory polymer(s); wherein: the first additive (a) comprises or is (optionally, is) a plurality of inorganic particles, (b) increases an ultrasound-attenuation characteristic of the composite material compared to that of the shape memory polymer(s) alone, and/or (c) comprises or is (optionally, is) a plurality of hollow particles; the composite material is characterized by the composite transition temperature (T_{cm,trans}); and the one or more portions of the composite material undergo the shape change from a temporary shape to a permanent shape when the one or more portions are heated to within 35° C. (optionally within 30° C., optionally within 25° C., optionally within 20° C., optionally within 15° C., optionally within 10° C., optionally within 5° C.) of T_{cm,trans} or a temperature approximately equal to (e.g., “approximately equal”=within 20%, optionally within 10%, optionally within 5%, optionally within 1%, optionally equal) or greater than T_{cm,trans}.

Aspects disclosed herein include a method of making a device having a composite material, the method comprising: attaching, providing, or inserting the composite material to or into the device; wherein the composite material comprises: one or more shape memory polymers; and a first additive provided in the shape memory polymer(s); wherein: the first additive (a) comprises or is (optionally, is) a plurality of inorganic particles, (b) increases an ultrasound-attenuation characteristic of the composite material compared to that of the shape memory polymer(s) alone, and/or (c) comprises or is (optionally, is) a plurality of hollow particles; the composite material is characterized by the composite transition temperature (T_{cm,trans}); and the one or more portions of the composite material undergo the shape change from a temporary shape to a permanent shape when the one or more portions are heated to within 35° C. (optionally within 30° C., optionally within 25° C., optionally within 20° C., optionally within 15° C., optionally within 10° C., optionally within 5° C.) of T_{cm,trans} or a temperature approximately equal to (e.g., “approximately equal”=within 20%, optionally within 10%, optionally within 5%, optionally within 1%, optionally equal) or greater than T_{cm,trans}.

Aspects disclosed herein include a device comprising a composite material, wherein the composite material comprises: one or more shape memory polymers; and a first additive provided in the shape memory polymer(s); wherein: the first additive (a) comprises or is (optionally, is) a plurality of inorganic particles, (b) increases an ultrasound-attenuation characteristic of the composite material compared to that of the shape memory polymer(s) alone, and/or (c) comprises or is (optionally, is) a plurality of hollow particles; the composite material is characterized by the composite transition temperature (T_{cm,trans}); and the one or more portions of the composite material undergo the shape change from a temporary shape to a permanent shape when the one or more portions are heated to within 35° C. (optionally within 30° C., optionally within 25° C., optionally within 20° C., optionally within 15° C., optionally within 10° C., optionally within 5° C.) of T_{cm,trans} or a temperature approximately equal to (e.g., “approximately equal”=within 20%, optionally within 10%, optionally within 5%, optionally within 1%, optionally equal) or greater than T_{cm,trans}.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIGS. 1A-1B: Reaction schemes for synthesis of crosslinked polycyclooctene.

FIG. 1A: General scheme. FIG. 1B: G2=Grubbs 2^{nd} generation catalyst. [Ru]=ruthenium based catalyst.

FIGS. 2A-2B: Schemes describing (FIG. 2A) synthesis of uncrosslinked poly(cyclooctene) via ring opening metathesis polymerization and (FIG. 2B) thermal crosslinking of poly(cyclooctene) with a thermal radical initiator, in this case dicumyl peroxide.

FIG. 3: Scheme and chart describing polymer synthesis. Fe₃O₄ nanoparticles are named by particle diameter. Additive density was obtained from product datasheets. ^{a}Melting temperature, Tₘ, and crystallization temperature, T_{c}, were obtained from differential scanning calorimetry using the second cycle heating or cooling curve, respectively.

FIG. 4: ¹H NMR spectrum of crosslinker.

FIG. 5: Scheme and chart describing polymer synthesis to make composites with varying additive and thixotrope. HGM=hollow glass microsphere. SGM=solid glass microsphere. Melting temperature, Tₘ, and crystallization temperature, T_{c}, were obtained from differential scanning calorimetry using the second cycle heating or cooling curve, respectively.

FIG. 6: Example DSC data used to determine T_{c} and Tₘ. Stability of the DSC results over 3 cycles beyond the 1^{st} cycle is noted.

FIG. 7A: Sample thickness vs. equilibration temperature for a sample with compressed temporary shape. FIG. 7B: DSC of a sample with the same thermal history and processing as in FIG. 7A.

FIG. 8A: Scheme and table showing polymerization conditions and Tₘ and T_{c} values measured by DSC. FIG. 8B: Plot of Tₘ vs. crosslinker loading.

FIG. 9: Shape memory demonstration. A compressed cylindrical sample expands in height and contracts in diameter to restore its permanent shape. Time zero is when the sample is first placed on a dish heated to 60° C.

FIG. 10: Custom laser cut clamp used to compress shape memory polymer samples.

FIG. 11A: Schematic description of the setup for ultrasound testing. FIG. 11B: Thermal camera temperature plotted vs. time for an ellipsoid region positioned on the sample. The temperature of the surrounding water did not change significantly from 37.0° C. FIG. 11C: Photographs of ultrasound testing of a folded shape memory polymer. Rapid unfolding is observed upon ultrasound exposure. “US”=ultrasound.

FIGS. 12A-12C: Summary of ultrasound response of materials with varying additive. FIG. 12A: Change in thickness before/after ultrasound of each sample. FIG. 12B: Maximum temperature reached during ultrasound exposure. FIG. 12C: Sample characteristics. Additive density was obtained from product datasheets. ^{a}Melting temperature, Tₘ, and crystallization temperature, T_{c}, were obtained from DSC using the second cycle heating or cooling curve, respectively.

FIGS. 13A-13D: Comparison of spatial heating response to ultrasound. (Left) Self-normalized thermal camera images taken after 150 seconds ultrasound exposure. (Right) photographs of samples before and during ultrasound exposure. “US”=ultrasound.

FIG. 14: Photographs of a flattened sheet (MK1-7C) upon ultrasound exposure and after exposure (right).

FIG. 15: Photographs of 90° bent samples after ultrasound exposure. Sample characteristics are shown. Tₘₐₓ refers to the maximum temperature measured during ultrasound exposure.

FIG. 16: Scheme and chart describing polymer synthesis to make composites with varying additive and thixotrope. HGM=hollow glass microsphere. SGM=solid glass microsphere. Melting temperature, Tₘ, and crystallization temperature, T_{c}, were obtained from differential scanning calorimetry using the second cycle heating or cooling curve, respectively.

FIG. 17A: Scheme and table showing crosslinking conditions and Tₘ and T_{c} values measured by DSC. FIG. 17B: Plot of Tₘ vs. crosslinker loading. The 3 wt. % point was omitted from regression due to significant broadening in the DSC melting curve compared to other samples.

FIG. 18A: Scheme and table showing crosslinking conditions and Tₘ and T_{c} values measured by DSC. FIG. 18B: Plot of Tₘ and T_{c} vs. crosslinker loading. HGM=hollow glass microsphere.

FIG. 19: DSC heating and cooling curves (2^{nd} cycle) for times of 2 h, 4 h, 8 h, and 24 h crosslinking in the vacuum oven at 140° C. Arrows indicate the direction of evolution of signal over the course of the crosslinking reaction. Inset table summarizes changes in melting/crystallization over time.

FIG. 20: DSC heating and cooling curves (2^{nd} cycle) for polymers without additive crosslinked with DCP or DBzP. Approximately the same thermogram was obtained for both samples.

FIG. 21: Specific heat capacity of Vestenamer 8012 as a function of temperature.

FIG. 22: Average ultrasound transmission of Vestenamer 8012 as a function of sample thickness.

FIG. 23A: Photograph of tensile testing setup with speckled sample used for digital image correlation (DIC). FIG. 23B: Stress vs. time plot for same specimen with reported yield stress (dashed line) of Vestenamer 8012. FIG. 23C: Stress vs. strain with linear regression.

FIG. 24: Thermal camera image of sample as clamped on edge of hot plate. Temperature vs. distance (cm) from the hotplate along a line down the center of the sample. Simulated data is overlaid.

FIG. 25A: Table describing sample characteristics and results of ultrasound test. PS=permanent shape thickness, TS=temporary shape thickness, AUS=after ultrasound thickness, Δt=change in thickness, Rᵣ(1)=strain recovery ratio; a value of 1 indicates complete shape recovery resulting from ultrasound, ΔT=change in temp. during the test defined as maximum temp. reached—temp. before ultrasound exposure. FIG. 25B: Rᵣ(1) and ΔT vs. K25 HGM loading. FIG. 25C: Heating kinetic profile for samples containing 5.0% or 0.5% of K25 HGMs. Ultrasound turned on at 30 s and off at 90 s.

FIG. 26A: Table describing sample characteristics and results of ultrasound test. Δt=change in thickness, Δd=change in diameter, R_{r,t}(1)=strain recovery ratio in thickness, R_{r,d}(1)=strain recovery ratio in diameter; a value of 1 indicates complete strain recovery resulting from ultrasound, ΔT=change in temp. during the test defined as maximum temp. reached—temp. before ultrasound exposure, t(50° C.)=time after turning on ultrasound to reach 50° C. FIG. 26B: Plot of ΔT for each additive. FIG. 26C: Properties of HGMs (3M Glass Bubbles) used in the study as obtained from manufacturer data sheets (3M).

FIGS. 27A-27D: Kinetic heating profiles for composites containing (FIG. 27A) K25 HGMs, (FIG. 27B) S35 HGMs, (FIG. 27C) iM30K HGMs, or (FIG. 27D) no additive exposed to ultrasound (670 kHz, 2.7 W cm⁻²).

FIGS. 28A-28D: Kinetic heating profiles for composites containing (FIG. 28A) <125 μm SGMs, (FIG. 28B) 212-300 μm SGMs, (FIG. 28C) 425-600 μm SGMs, or (FIG. 28D) 1000 μm SGMs exposed to ultrasound (670 kHz, 2.7 W cm⁻²).

FIG. 29A: Photograph and thermal image (FIG. 29B) of sample in ultrasound testing setup before ultrasound exposure. FIG. 29C: Photograph and thermal image (FIG. 29D) of sample during ultrasound exposure (670 kHz, 2.7 W cm⁻²) after shape change is complete. FIG. 29E: Kinetic heating profile during the experiment (670 kHz, 2.7 W cm⁻²). Ultrasound was turned on at t=20 s and off at t=40 s.

FIGS. 30A-30C: Simulation 1. Simulated temperature response to focused ultrasound in polymer containing SGMs (left), no additive (middle), or air bubbles (right) for additives of size 300 μm (FIG. 30A), 100 μm (FIG. 30B), and 50 μm (FIG. 30C). The polymer is surrounded by tissue as in a medical device application.

FIG. 31A: Simulation parameters. FIG. 31B: Mean temperature (Simulation 1) reached in polymer matrix in response to focused ultrasound in samples containing solid glass microspheres (SGMs), no additives, or air bubbles. FIG. 31C: Same as B, for Simulation 2, where there is a tissue/air interface (instead of all tissue as in Simulation 1) to more closely match experimental setup.

FIGS. 32A-32C: Simulation 2. Simulated temperature response to focused ultrasound in polymer containing SGMs (left), no additive (middle), or air bubbles (right) for additives of size 300 μm (FIG. 32A), 100 μm (FIG. 32B), and 50 μm (FIG. 32C). This simulation invokes a layer of air surrounding the polymer except on the bottom, where it is in contact with tissue, to approximate experimental setups.

### STATEMENTS REGARDING CHEMICAL COMPOUNDS AND NOMENCLATURE

In general, the terms and phrases used herein have their art-recognized meaning, which can be found by reference to standard texts, journal references and contexts known to those skilled in the art. The following definitions are provided to clarify their specific use in the context of the invention.

The term “shape memory polymer” (or, “SMP”) refers to a polymer that has the ability to return from a deformed shape (a temporary shape) or state to its original shape (a permanent shape) or state when induced by an external stimulus, such as temperature change. This behavior is also referred to as a shape memory behavior. The permanent shape of a shape memory polymer or of a composite material therewith is set, established, or defined upon the formation of chemical or physical crosslinks therein. For example, the crosslinking can occur at a temperature approaching (e.g., within 20%), equal to, or greater than a T_{crosslink}, or a crosslinking temperature. For example, a permanent shape can be imparted upon or forced upon a shape memory polymer or a composite material therewith when or as crosslinking is occurring therein (at a temperature approaching, near, at, or beyond T_{crosslink}) by constraining at least a portion of the polymer or material (such as in a mold), applying a force to at least a portion of the polymer or material, and/or applying tension or compression to the polymer or material, or mechanically or otherwise deforming the polymer or material during crosslinking or as crosslinking occurs. A temporary shape of a shape memory polymer or a composite material therewith can be set by heating the polymer or material to its transition temperature, Tₜᵣₐₙₛ, which may correspond to the polymer's or material's glass transition temperature, T_{g}, and/or its melt temperature, Tₘ. As the temperature approaches (e.g., within 20%), is equal to, or is greater than Tₜᵣₐₙₛ, the polymer or material can be deformed, optionally mechanically, to establish or set the temporary shape. As the deformation, corresponding to the temporary shape, is applied to the polymer or material, the deformation is preferably maintained, mechanically or otherwise, as the polymer or material is cooled to below its Tₜᵣₐₙₛ, thereby setting or defining the temporary shape of the polymer or material. Crystallization of ordered domains and/or vitrification (where Tₜᵣₐₙₛ=Tₘ and/or T_{g}, respectively) serves to hold the polymer in its temporary shape until it is again heated to a temperature approaching, equal to, or greater than Tₜᵣₐₙₛ, in which case, in the absence of the deformation (e.g., mechanical force) which established the temporary shape, the polymer or material will spontaneously return to its permanent shape. This transformation in form is known as the shape memory effect.

As used herein, a “shape” of a material refers to its geometry or physical configuration. In aspects, the term “shape” refers to a material's macroscopic geometry or physical configuration as well as one or more microscopic, nanoscopic, and/or atomic physical characteristics, including but not limited to strain and/or stress. Therefore, as used herein, a “shape change” refers to a change in a material's macroscopic geometry or physical configuration and optionally further includes change in one or more microscopic, nanoscopic, and/or atomic physical characteristics, such as a distribution in stress and/or strain in the material. As merely an illustrative example, if a shape memory polymer or composite material therewith is programmed to contract as part of the shape change from temporary shape to permanent shape but is fixed on both ends during the shape change (e.g., as the material is heated to Tₜᵣₐₙₛ, or T_{cm,trans} in case of composite material), the shape change may be more subtle or limited to a change in forces within the structure. In various aspects, a shape change is an expansion, a contraction, a twisting, an unraveling, a curling, an unfurling, an opening, a closing, a bending, an unbending, a folding, an unfolding, a straightening, a lengthening, a shortening, a redistribution or change in distribution of stress in the material, a redistribution or change in distribution of strain in the material, or any combination of these. In some aspects, a shape change is not necessarily from a temporary shape to the final/original permanent shape, but instead the shape change is complete or stopped at an intermediate shape (e.g., an intermediate shape between temporary shape and permanent shape before original permanent shape is obtained).

The term “transition temperature” or “Tₜᵣₐₙₛ” refers to a characteristic temperature of the phase or shape change corresponding to a shape memory polymer or a composite material having a shape memory polymer, or any portion(s) thereof, undergoing or exhibiting a shape memory effect, a shape change, or otherwise a transformation from its temporary shape to its permanent shape when heated from a lower temperature thereto. In some aspects, a transition temperature of a material corresponds to a glass transition temperature and/or melt transition temperature of the material. In some aspects, a material may have two transition temperatures, a glass transition and a melt transition, wherein only the melt transition lies above room temperature, enabling formation of a temporary shape that is stable at room temperature. For example, in aspects, assuming endotherms are plotted as negative, the transition temperature for the melt transition corresponds to the minimum of the negative peak in the 2^{nd} cycle heating curve in differential scanning calorimetry (DSC) data. In aspects, for determining a Tₜᵣₐₙₛ, when plotting DSC data with the positive exotherm convention, as provided herein, for example, the heating curve upon melting (endothermic) will have a negative peak and Tₘ is defined as the minimum. Use of DSC for determining relevant transition temperature(s), useful for aspects disclosed herein, such as Tₘ, T_{c}, and/or T_{g} is described by Gabbott, P. in “A Practical Introduction to Differential Scanning Calorimetry”, Chapter 1, pp. 1-50, in Principles and Applications of Thermal Analysis, 2008 (DOI: 10.1002/9780470697702.ch1), especially section 1.5.2.3 and 1.5.5, which are incorporated herein by reference in their entireties. A composite material, having one or more shape memory polymers, optionally further comprising one or more additives, can be characterized by a transition temperature of the composite material, for which the shorthand used herein is T_{cm,trans}. A crosslinked shape memory polymer that is free of additives disclosed herein, or an “additive-free crosslinked shape memory polymer”, can be characterized by a transition temperature of the additive free crosslinked shape memory polymer, or a polymer-only transition temperature, or simply T_{pol,trans}.

The terms “crosslinking temperature” and “T_{crosslink}” refer to a characteristic onset or a lower limit temperature of chemical and/or physical crosslinking in a shape memory polymer or a composite material having a shape memory polymer, or any portion(s) thereof, corresponding to the setting, programming, or establishing of its permanent shape when heated from a lower temperature thereto. As used herein, the term “T_{crosslink}” refers to the lower limit or onset temperature of chemical crosslinking as just defined, whereas “Tₚₑᵣₘ” (or, permanent shape programming temperature) refers to the temperature that is actually used or reached for crosslinking and programming/setting the permanent shape. Thus, Tₚₑᵣₘ is equal to or greater than T_{crosslink}. Tₚₑᵣₘ is not necessarily an intrinsic property of the material, but rather depends on the crosslinker (or, crosslinking moiety, or crosslinking precursor) used, and is chosen depending on practical considerations such as, but not limited to, a reasonable reaction time, temperature limits of the mold used, and avoiding decomposition of the SMP. For example, in aspects, a crosslinking temperature corresponds to a temperature at which a thermal initiator decomposes, causing the crosslinking reaction to occur. In some aspects, T_{crosslink} is not necessarily directly measured, but a valid lower limit for Tₚₑᵣₘ, and thus an estimate of T_{crosslink}, can be estimated. For example, in aspects, an upper limit of Tₚₑᵣₘ or range thereof could be defined as the temperature at which the SMP and/or crosslinking moieties thereof decompose. The crosslinking reaction will occur faster the higher the temperature above the lower limit T_{crosslink}. As such, as used herein, Tₚₑᵣₘ corresponds to the lower limit temperature, or onset temperature, of the crosslinking reaction in a given polymer or material. Once the crosslinking step has been completed, (re)heating to T_{crosslink}, Tₚₑᵣₘ, or higher no longer elicits crosslinking or shape programming effect, since the crosslinker has been completely consumed. Generally, heating to T_{crosslink} or Tₚₑᵣₘ does not trigger any shape change in a crosslinked SMP. The minimum time required at a particular given temperature, being approximately equal to or greater than T_{crosslink}, to complete crosslinking can be estimated by a kinetic study where samples of the material are heated to that particular temperature Tₚₑᵣₘ for varying amounts of time. DSC can be used for such a study (see FIG. 19), for example, where bimodal peaks (Tₘ and T_{c}) indicate incomplete crosslinking. The time at which the peaks stabilize at their final shapes, and no longer change, indicates the minimum crosslinking time at a particular temperature at which crosslinking is performed. A composite material having one or more shape memory polymers and further comprising one or more additives, according to aspects disclosed herein, or a monomeric formulation capable of forming the composite material and comprising crosslinking precursor(s) and one or more additives can be characterized by a minimum/onset/lower limit crosslinking temperature of the composite material, for which the shorthand used herein is T_{cm,crosslink}. An actual temperature used to crosslink and program/set a permanent shape for a composite material having one or more shape memory polymers and further comprising one or more additives, according to aspects disclosed herein, or a monomeric formulation capable of forming the composite material and comprising crosslinking precursor(s) and one or more additives may be referred to herein as the permanent shape programming temperature or T_{cm,perm}. In contrast to a composite material having one or more additives, as disclosed herein, an additive-free shape memory polymer comprising crosslinking precursor(s) or an additive-free monomeric formulation having crosslinking precursor(s) and capable of forming a shape memory polymer can be characterized by a minimum/onset/lower limit crosslinking temperature, or simply T_{pol,crosslink}. An actual temperature used to crosslink and program/set a permanent shape for an additive-free shape memory polymer comprising crosslinking precursor(s) or an additive-free monomeric formulation having crosslinking precursor(s) and capable of forming a shape memory polymer may be referred to herein as the permanent shape programming temperature or T_{pol,perm}. For example, in some aspects, crosslinking and permanent shape programming/setting of a composite with DBzP as crosslinking precursor is performed, in some aspects, at 140° C. (Tₚₑᵣₘ) for 8-12 hours, whereas a practical lower limit of crosslinking temperature (T_{crosslink}) of the formulation/mixture may be 91° C., and, in some aspects, a 10-hour HLT is used as lower limit of crosslinking temperature (T_{crosslink}) which may be 73° C. in the case of DBzP for example.

The term “10-hour half-life temperature” or “10-hour HLT” refers to a temperature at which 50% of the substance, compound, or material, such as an organic peroxide crosslinking precursor, will decompose in 10 hours. This parameter corresponds to an optional estimate of lower limit for estimating crosslinking temperature, T_{crosslink}, because a typical crosslinking reaction may be less efficient in a polymer in practice, since the crosslinker is spread out in the polymer matrix in solid state, compared to the 10-hour HLT measurement for a pure crosslinking precursor (e.g., organic peroxide) dissolved in an organic solvent. Relevant 10-hour HLT values for various compounds and descriptions of measurements thereof are available in “Kirk-Othmer Encyclopedia of Chemical Technology”, chapter “PEROXIDES AND PEROXIDE COMPOUNDS, ORGANIC PEROXIDES” (DOI: 10.1002/0471238961.1518070119011403.a01).

As used herein, the term “ultrasound” is intended to be consistent with the term as used in the field of physics. Generally, ultrasound is sound, sound frequencies, sound waves, or acoustic energy characterized by frequencies greater than approximately 20 kHz. In some aspects, ultrasound is characterized by sound wave frequencies of at least approximately 20 kHz and optionally less than or equal to 5 GHz, optionally less than or equal to 1 GHz, optionally less than or equal to 500 MHz, optionally less than or equal to 400 MHz, optionally less than or equal to 300 MHz, optionally less than or equal to 250 MHz, optionally less than or equal to 200 MHz, optionally less than or equal to 150 MHz, optionally less than or equal to 100 MHz, optionally less than or equal to 50 MHz, optionally less than or equal to 30 MHz, optionally less than or equal to 20 MHz, optionally less than or equal to 19 MHz, optionally less than or equal to 18 MHz, optionally less than or equal to 16 MHz, optionally less than or equal to 15 MHz optionally less than or equal to 14 MHz, optionally less than or equal to 10 MHz, optionally less than or equal to 9 MHz, optionally less than or equal to 5 MHz, optionally less than or equal to 4 MHz, optionally less than or equal to 3 MHz, optionally less than or equal to 1 MHz, optionally less than or equal to 900 kHz, optionally less than or equal to 800 MHz, optionally less than or equal to 700 MHz, optionally less than or equal to 600 MHz, optionally less than or equal to 500 MHz. In certain aspects or embodiments herein, the ultrasound frequency range is that which is relevant and useful to a given or specified application or field of applications (e.g., medical devices). Generally, the terms “ultrasound”, “ultrasound frequencies”, “ultrasound waves”, and “ultrasound energy” may be used interchangeably such as when referring to absorption of ultrasound energy by a material or to a beam of ultrasound energy/waves.

The term “focused ultrasound” refers to non-ionizing ultrasound, ultrasound frequencies/waves, or ultrasound energy that is directional and focused or confined. Focused ultrasound may focused or confined to a beam of ultrasound waves or ultrasound energy. A focused ultrasound beam may be characterized by a focal volume, focal area, or focal point at which the ultrasound intensity, energy density, power, power density, and/or a flux of the ultrasound beam is maximum. A focused ultrasound beam may be formed with the aid of a transducer. Focused ultrasound may be used for therapeutic techniques such as high-intensity focused ultrasound (HIFU).

The term “ultrasound-attenuation characteristic” refers to an empirically-derived or a computationally determined characteristic of a material that quantitatively describes or defines the ability of the material to attenuate ultrasound energy or ultrasound frequencies. An “ultrasound-absorption characteristic” is an ultrasound-attenuation characteristic. The term “ultrasound-absorption characteristic” refers to an empirically-derived or a computationally determined characteristic of a material that quantitatively describes or defines the ability of the material to absorb ultrasound energy or ultrasound frequencies. Ultrasound-attenuation characteristics include, but are not limited to, an ultrasound attenuation coefficient, an ultrasound absorptivity (e.g., molar absorptivity), an ultrasound absorption coefficient (e.g., mass absorption coefficient), and an ultrasound absorption cross-section. In some aspects, an ultrasound-attenuation characteristic is preferably an ultrasound attenuation coefficient.

The term “size characteristic” refers to a property, or set of properties, of a particle that directly or indirectly relates to a size attribute. According to some aspects, a size characteristic corresponds to an empirically-derived size characteristic of a particle or particles being detected, such as a size characteristic based on, determined by, or corresponding to data from any technique or instrument that may be used to determine a particle size, such as but not limiting to optical microscope, electron microscope (e.g., SEM and TEM), optical attenuation (absorbance, scattering, and/or reflectance), and/or a light scattering technique (e.g., DLS). For example, a size characteristic can correspond to a spherical particle exhibiting similar or substantially same properties, such as aerodynamic, hydrodynamic, optical, and/or electrical properties, as the particle(s) being detected. According to some aspects, a size characteristic corresponds to a physical dimension, such as a cross-sectional size (e.g., length, width, thickness, or diameter).

The terms “substantially” and “approximately” interchangeably refer to a property, condition, or value that is within 20%, 10%, within 5%, within 1%, optionally within 0.1%, or is equal to a reference property, condition, or value, respectively. The term “substantially equal”, “substantially equivalent”, or “substantially unchanged”, when used in conjunction with a reference value (e.g., describing a property or condition), refers to a value that is within 20%, within 10%, optionally within 5%, optionally within 1%, optionally within 0.1%, or optionally is equal to the provided reference value. For example, a temperature is substantially equal to 100° C. (or, “is substantially 100° C.” or “is approximately 100° C.”) if the value of the temperature is within 20%, optionally within 10%, optionally within 5%, optionally within 1%, within 0.1%, or optionally equal to 100° C. The term “substantially greater”, when used in conjunction with a reference value, refers to a value that is at least 1%, optionally at least 5%, optionally at least 10%, or optionally at least 20% greater than the provided reference value. The term “substantially less”, when used in conjunction with a reference value refers to a value that is at least 1%, optionally at least 5%, optionally at least 10%, or optionally at least 20% less than the provided reference value. As used herein, the terms “substantially” and “approximately” are equivalent and interchangeable. As used herein, the term “about” means a range of values including the specified value, which a person of ordinary skill in the art would consider reasonably similar to the specified value. In embodiments, “about” means within a standard deviation using measurements generally acceptable in the art. In embodiments, “about” means a range extending to ±10% of the specified value. In embodiments, “about” means the specified value.

The term “moiety” refers to a group, such as a functional group, of a chemical compound or molecule. A moiety is a collection of atoms that are part of the chemical compound or molecule. Crosslinking moieties disclosed herein include moieties characterized as monovalent, divalent, trivalent, etc. valence states, or any combination thereof. Generally, but not necessarily, a moiety comprises more than one functional group.

The term “NTP” refers to the set of conditions defined as normal temperature and pressure, which are a temperature of 20° C. (293.15 K, 68° F.) and an absolute pressure of 1 atm (14.696 psi, 101.325 kPa).

The term “wt. %” refers to a weight percent, or a mass fraction represented as a percentage by mass. As used herein, a concentration of the first additive in the one or more shape memory polymers may be characterized using a wt. % of the first additive in the one or more shape memory polymers, wherein wt. %=[(weight of first additive)/(weight of the one or more polymers only, excluding first additive)].

In an embodiment, a composition or compound of the invention, such as an alloy or precursor to an alloy, is isolated or substantially purified. In an embodiment, an isolated or purified compound is at least partially isolated or substantially purified as would be understood in the art. In an embodiment, a substantially purified composition, compound or formulation of the invention has a chemical purity of 95%, optionally for some applications 99%, optionally for some applications 99.9%, optionally for some applications 99.99%, and optionally for some applications 99.999% pure.

### DETAILED DESCRIPTION OF THE INVENTION

In the following description, numerous specific details of the devices, device components and methods of the present invention are set forth in order to provide a thorough explanation of the precise nature of the invention. It will be apparent, however, to those of skill in the art that the invention can be practiced without these specific details.

Various aspects herein are in the fields of stimulus responsive materials and shape memory polymers and their use in medical devices. In some aspects, the stimulus is ultrasound and various aspects are in the field of ultrasound responsive shape memory polymers. Composite materials, in various aspects, comprise one or more shape memory polymers of which a shape change can be triggered through the application of ultrasound. In aspects herein, composite materials further comprise one or more additives that improve or enhance the ability of the composite material or SMP thereof to absorb ultrasound energy, thereby triggering a shape change without damaging nearby tissue. In various aspects, composite materials are designed with control of Tₜᵣₐₙₛ to fall below, at, or above body temperature (T=37° C.) to enable their use in medical applications.

The shape memory process can be used to perform a desired function as a component of a medical device or tool. For example, shape memory polymers have been used for drug delivery,² to make self-tightening sutures,³ for auto-expanding stents,⁴ and for other biomedical applications.¹ However, most existing applications make use of heat as the stimulus to trigger a shape memory effect, with relatively few examples of other stimuli.

The external stimuli used to actuate shape memory polymers include heat, light, ultrasound, magnetic fields, resistive heating, pH change, and chemical exposure.¹ Of these, only ultrasound, magnetic fields, and pH changes are capable of noninvasively activating a SMP that is part of a medical implant. Heating is unspecific and will cause damage to neighboring tissues. Light cannot penetrate significantly beyond the skin. Chemical exposure introduces significant toxicity concerns. In contrast, focused ultrasound can be used deep within the body to generate localized heating and is in widely used for the ablation of tumors.⁵ This use has been applied clinically in humans⁶ and the associated equipment can be adapted to actuate a shape memory polymer.

Achieving synthetic control over Tₜᵣₐₙₛ is desirable to precisely tailor an SMP to a particular medical application. For most medical applications, a Tₜᵣₐₙₛ that is tunable over a range of temperatures spanning the physiological temperature is desirable. Poly(cis-cyclooctene) (PCOE) is a promising polymer for achieving control over Tₜᵣₐₙₛ in that it has been demonstrated to show tunable Tₜᵣₐₙₛ (in this case Tₘ) in the desired range as a function of cis/trans ratio of alkene moieties in the polymer backbone.¹⁰ In addition, the Tₘ has been tuned through varying the loading of the crosslinker dicumyl peroxide, where higher loadings were found to decrease Tₘ.¹¹ However, in these examples, ultrasound actuation was not realized. Thus, development of a PCOE based SMP that can be activated via ultrasound would be highly desirable to realize a system where Tₘ is tunable.

Finally, to increase the safety margin within the human body, the SMP should demonstrate significantly stronger absorption of ultrasound than surrounding bone and tissue.¹² Composite materials such as Teflon coated NaCl crystals embedded in a polymer matrix have been shown to generate very significant heating effects upon ultrasound stimulation.¹³ The heating effect was attributed to friction at the interface between the Teflon coating and the polymer. However, this effect has not been used to trigger shape memory, and high pressure was required in addition to ultrasound in this setup, rendering it unsuitable for medical use. As such, development of a composite SMP material that is suitable for medical use represents a promising strategy to increase the safety margin of an ultrasound responsive SMP.

In various aspects, composite materials comprise a semicrystalline shape memory polymer which comprises a chemically and/or physically crosslinked network and contains one or more additives. For example, an additive may be intentionally introduced pockets of gas in the SMP and/or intentionally introduced materials different from the SMP itself. Composite materials of aspects disclosed herein, also referred to herein as “polymer/additive composites” or simply “composites”, are characterized by enhanced ultrasound induced heating as compared with the equivalent crosslinked shape memory polymer free of additives (additive-free crosslinked shape memory polymer). Composite materials of aspects disclosed herein demonstrate a shape memory effect upon heating to T_{cm,trans} which may also be triggered through the application of an external stimulus. Both the permanent and temporary shapes can be fully programmed through synthesis and processing and are not limited to any particular geometry.

In various aspects, the permanent shape is set at the time or occurrence of crosslink formation, while the temporary shape is defined through later processing of the already crosslinked polymer. An irreversible shape memory effect, or an irreversible shape change, is a shape change from the temporary shape to the permanent shape upon application of the external stimulus but where the temporary shape is not recovered upon removal or turning off of the external stimulus (e.g., focused ultrasound). A reversible shape memory effect, or a reversible shape change, is a shape change between temporary shape and permanent shapes as the external stimulus is turned on and off, such that the temporary shape may be recovered when the external stimulus is removed or turned off. In some embodiments, the external stimulus is ultrasound, light, chemical exposure, magnetic or electric field exposure, or pH change.

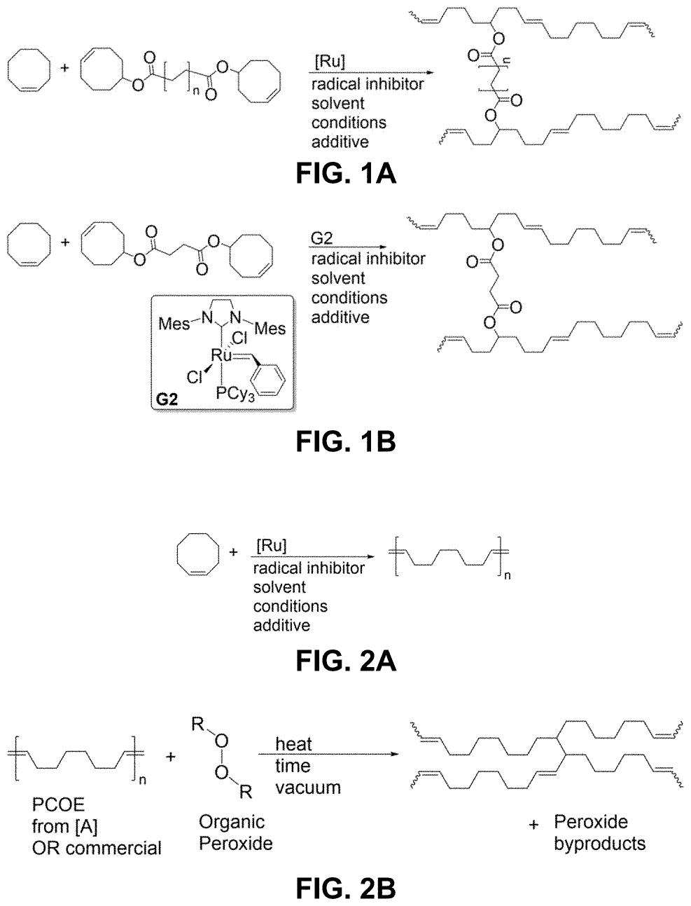

In various aspects, the semicrystalline polymer is poly(cyclooctene) prepared via ring opening metathesis polymerization (ROMP) with a ruthenium catalyst and crosslinked chemically in a single step by a difunctional cyclooctene crosslinker (FIG. 1A). In various aspects, the catalyst is G2, and the cyclooctene moieties of the crosslinker are separated by 2 carbons (n=2, FIG. 1B). In various aspects, the semicrystalline polymer is poly(cyclooctene) prepared via ring opening metathesis polymerization (ROMP) with a ruthenium catalyst and crosslinked in a second step with a thermally activated radical initiator (FIG. 2A). The poly(cyclooctene) may also be purchased commercially (example: Vestenamer 8012, Evonik) and crosslinked in a second step with a thermally activated radical initiator (FIG. 2B). Prior to crosslinking at elevated temperature, poly(cyclooctene) containing a thermally activated radical initiator may be drawn into filament and extruded using 3D-printing.

In various aspects, Vestenamer 8012 (Evonik) is crosslinked with dicumyl peroxide (DCP) or dibenzoyl peroxide (DBzP). The polymer is dissolved in toluene with the crosslinker and may be mixed with an additive to enhance ultrasound induced heating. After removing the solvent and processing the dried polymer into the desired shape, crosslinking is achieved by exposure to elevated temperature under vacuum (FIGS. 2A-2B).

In various aspects, a thermal radical initiator may be included as an additive within a crosslinked polymer prepared according to aspects herein to be activated at a later time directly through exposure to elevated temperature or indirectly through exposure to an external stimulus. Elevated temperature or the external stimulus is used to form an interpenetrating second crosslinked network while the polymer is held mechanically in a temporary shape. The interpenetrating network may be used to enable a reversible shape memory effect.

In various aspects, monomer may be introduced via swelling into the polymer containing thermal initiator prepared according to aspects herein, and the use of elevated temperature or an external stimulus will induce radical polymerization.

In various aspects, the polymer is prepared according to aspects herein and the thermal radical initiator is DCP and the external stimulus is focused ultrasound stimulation.

### CERTAIN ASPECTS AND EMBODIMENTS

Various aspects are contemplated herein, several of which are set forth in the paragraphs below. It is explicitly contemplated that any aspect or portion thereof can be combined to form an aspect. In addition, it is explicitly contemplated that: any reference to aspect 1 includes reference to aspects 1a, 1b, 1c, 1d, 1e, 1f, 1g, and/or 1h, etc., and any combination thereof; any reference to aspect 2 includes reference to aspects 2a, 2b, and 2c, and so on (any reference to an aspect includes reference to that aspect's lettered versions). Moreover, the terms “any preceding aspect” and “any one of the preceding aspects” means any aspect that appears prior to the aspect that contains such phrase (for example, the sentence “Aspect 32: The method or system of any preceding aspect . . . ” means that any aspect prior to aspect 32 is referenced, including letter versions, including aspects 1a through 31). For example, it is contemplated that, optionally, any material, method, or device of any the below aspects may be useful with or combined with any other aspect provided below. Further, for example, it is contemplated that any embodiment or aspect described above may, optionally, be combined with any of the below listed aspects.

Aspect 1a: A composite material comprising:
one or more shape memory polymers; and
a first additive provided in the shape memory polymer(s); wherein:
the first additive comprises or is (optionally, is) a plurality of inorganic particles; the composite material is characterized by a composite transition temperature (T_{cm,trans}); and
the composite material or one or more portions thereof undergo a shape change from a temporary shape to a permanent shape when the composite material or said one or more portions thereof are heated to within 35° C. of T_{cm,trans} or a temperature approximately equal to or greater than T_{cm,trans}.

Aspect 1b: A composite material comprising:
one or more shape memory polymers; and
a first additive provided in the shape memory polymer(s); wherein:
the first additive increases (optionally by at least 25%, optionally by at least 50%, optionally by at least 75%, optionally by at least 100%, optionally by at least 150%, optionally by at least 175%, optionally by at least 200%) one or more ultrasound-attenuation characteristics of the composite material (or at least of one or more portions thereof having the first additive) same one or more shape memory polymers free of said first additive;
the composite material is characterized by a composite transition temperature (T_{cm,trans}); and
the composite material or one or more portions thereof undergo a shape change from a temporary shape to a permanent shape when the composite material or said one or more portions thereof are heated to within 35° C. (optionally within 30° C., optionally within 25° C., optionally within 20° C., optionally within 15° C., optionally within 10° C., optionally within 5° C.) of T_{cm,trans} or a temperature approximately equal to (e.g., “approximately equal”=within 20%, optionally within 10%, optionally within 5%, optionally within 1%, optionally equal) or greater than T_{cm,trans}.

Aspect 1c: A composite material comprising:
one or more shape memory polymers;
a first additive provided in the shape memory polymer(s); wherein:
the first additive comprises or is (optionally, is) a plurality of hollow particles;
the composite material is characterized by a composite transition temperature (T_{cm,trans}); and
the composite material or one or more portions thereof undergo a shape change from a temporary shape to a permanent shape when the composite material or said one or more portions thereof are heated to within 35° C. of T_{cm,trans} or a temperature approximately equal to or greater than T_{cm,trans}.

Aspect 1d: A method of using a composite material, the method comprising:
directing one or more focused ultrasound beams at one or more portions of the composite material;
thereby, heating the one or more portions to a temperature approximately equal to or greater than a composite transition temperature (T_{cm,trans}); and
thereby, causing the composite material to undergo a shape change at the one or more portions thereof;
wherein the composite material comprises:
   one or more shape memory polymers; and
   a first additive provided in the shape memory polymer(s); wherein:
   the first additive (a) comprises or is (optionally, is) a plurality of inorganic particles, (b) increases an ultrasound-attenuation characteristic of the composite material (or at least of one or more portions thereof having the first additive) compared to same one or more shape memory polymers free of said first additive, and/or (c) comprises or is (optionally, is) a plurality of hollow particles;
   the composite material is characterized by the composite transition temperature (T_{cm,trans}); and
   the one or more portions of the composite material undergo the shape change from a temporary shape to a permanent shape when the one or more portions are heated to within 35° C. (optionally within 30° C., optionally within 25° C., optionally within 20° C., optionally within 15° C., optionally within 10° C., optionally within 5° C.) of T_{cm,trans} or a temperature approximately equal to (e.g., “approximately equal”=within 20%, optionally within 10%, optionally within 5%, optionally within 1%, optionally equal) or greater than T_{cm,trans}.

Aspect 1e: A method of making a composite material, the method comprising:
polymerizing a monomer to form a first polymer;
crosslinking the first polymer in the presence of a crosslinking precursor and the first additive at a temperature approximately (e.g., within 20%) equal to or greater than a crosslinking temperature (T_{cm,crosslink}) (optionally at a temperature greater than or equal to 65° C., optionally a temperature greater than or equal to 75° C., a temperature greater than or equal to 85° C., a temperature greater than or equal to 90° C., a temperature greater than or equal to 95° C., a temperature greater than or equal to 100° C., a temperature greater than or equal to 105° C., a temperature greater than or equal to 110° C., a temperature greater than or equal to 115° C.) to form the composite material having the crosslinked shape memory polymer and the first additive;
wherein the composite material comprises:
   one or more shape memory polymers; and
   a first additive provided in the shape memory polymer(s); wherein:
   the first additive (a) comprises or is (optionally, is) a plurality of inorganic particles, (b) increases an ultrasound-attenuation characteristic of the composite material (or at least of one or more portions thereof having the first additive) compared to same one or more shape memory polymers free of said first additive, and/or (c) comprises or is (optionally, is) a plurality of hollow particles;
   the composite material is characterized by the composite transition temperature (T_{cm,trans}); and
   the one or more portions of the composite material undergo the shape change from a temporary shape to a permanent shape when the one or more portions are heated to within 35° C. (optionally within 30° C., optionally within 25° C., optionally within 20° C., optionally within 15° C. optionally within 10° C., optionally within 5° C.) of T_{cm,trans} or a temperature approximately equal to (e.g., “approximately equal”=within 20%, optionally within 10%, optionally within 5%, optionally within 1%, optionally equal) or greater than T_{cm,trans}.

Aspect 1f: A method of making a device having a composite material, the method comprising:
attaching, providing, or inserting the composite material to or into the device;
wherein the composite material comprises:
   one or more shape memory polymers; and
   a first additive provided in the shape memory polymer(s); wherein:
   the first additive (a) comprises or is (optionally, is) a plurality of inorganic particles, (b) increases an ultrasound-attenuation characteristic of the composite material (or at least of one or more portions thereof having the first additive) compared to same one or more shape memory polymers free of said first additive, and/or (c) comprises or is (optionally, is) a plurality of hollow particles;
   the composite material is characterized by the composite transition temperature (T_{cm,trans}); and
   the one or more portions of the composite material undergo the shape change from a temporary shape to a permanent shape when the one or more portions are heated to within 35° C. (optionally within 30° C., optionally within 25° C., optionally within 20° C., optionally within 15° C. optionally within 10° C., optionally within 5° C.) of T_{cm,trans} or a temperature approximately equal to (e.g., “approximately equal”=within 20%, optionally within 10%, optionally within 5%, optionally within 1%, optionally equal) or greater than T_{cm,trans}.

Aspect 1g: A device comprising: a composite material, wherein the composite material comprises:
one or more shape memory polymers; and
a first additive provided in the shape memory polymer(s); wherein:
the first additive (a) comprises or is (optionally, is) a plurality of inorganic particles, (b) increases an ultrasound-attenuation characteristic of the composite material (or at least of one or more portions thereof having the first additive) compared to that of the shape memory polymer(s) alone, and/or (c) comprises or is (optionally, is) a plurality of hollow particles;
the composite material is characterized by the composite transition temperature (T_{cm,trans}); and
the one or more portions of the composite material undergo the shape change from a temporary shape to a permanent shape when the one or more portions are heated to within 35° C. (optionally within 30° C., optionally within 25° C., optionally within 20° C., optionally within 15° C., optionally within 10° C., optionally within 5° C.) of T_{cm,trans} or a temperature approximately equal to (e.g., “approximately equal”=within 20%, optionally within 10%, optionally within 5%, optionally within 1%, optionally equal) or greater than T_{cm,trans}.

Aspect 1 h: A composite material comprising:
one or more shape memory polymers; and
a first additive provided in the one or more shape memory polymers; wherein:
(a) the first additive comprises or is (optionally, is) a plurality of inorganic particles, (b) the first additive increases (optionally by at least 25%, optionally by at least 50%, optionally by at least 75%, optionally by at least 100%, optionally by at least 150%, optionally by at least 175%, optionally by at least 200%) an ultrasound attenuation coefficient of the composite material (or at least of one or more portions thereof having the first additive) compared to that of the same one or more shape memory polymers free of said first additive; and/or (c) the first additive comprises or is (optionally, is) a plurality of hollow particles;
the composite material is characterized by a composite transition temperature (T_{cm,trans});
the first additive is provided at least at one or more portions of the composite material; and
the composite material or the one or more portions thereof undergo a shape change from a temporary shape to a permanent shape when the composite material or said one or more portions thereof are heated to within 35° C. (optionally within 30° C., optionally within 25° C., optionally within 20° C., optionally within 15° C., optionally within 10° C., optionally within 5° C.) of T_{cm,trans} or a temperature approximately equal to (e.g., “approximately equal”=within 20%, optionally within 10%, optionally within 5%, optionally within 1%, optionally equal) or greater than T_{cm,trans}.

Aspect 2a: The composite material, method, and/or device of any preceding Aspect, wherein the first additive is provided at (or at least at) the one or more portions of the shape memory polymer(s). Aspect 2b: The composite material, method, and/or device of any preceding Aspect, wherein the first additive is provided at the one or more portions of the shape memory polymer(s); and wherein at least a portion of the one or more shape memory polymers is free of the first additive. Aspect 2c: The composite material, method, and/or device of any preceding Aspect, wherein the first additive is provided at (or at least at) one or more portions of the composite material. Aspect 2d: The composite material, method, and/or device of any preceding Aspect, wherein the first additive is provided at one or more portions of the composite material; and wherein at least another portion of the one or more shape memory polymers is free of the first additive.

Aspect 3: The composite material, method, and/or device of any preceding Aspect, wherein the first additive is provided throughout the internal volume of the shape memory polymer(s).

Aspect 4: The composite material, method, and/or device of any preceding Aspect being capable of absorbing ultrasound throughout or at the one or more portions of the composite material; wherein a temperature of the composite material increases where its absorbs the ultrasound; and wherein the composite material undergoes the shape change when heated to within 35° C. of T_{cm,trans} or a temperature approximately equal to or greater than T_{cm,trans} as a result of absorbing of the ultrasound.

Aspect 5: The composite material, method, and/or device of any preceding Aspect, wherein the one or more portions of the composite material having the first additive are heated when said one or more portions of the composite material are exposed to ultrasound.

Aspect 6: The composite material, method, and/or device of any preceding Aspect, wherein the first additive absorbs the ultrasound frequencies; and wherein the first additive is heated by its absorption of ultrasound and/or wherein heat is created by friction between the first additive and the one or more shape memory polymers when the first additive absorbs the ultrasound.

Aspect 7: The composite material, method, and/or device of any preceding Aspect, wherein the composite material undergoes the shape change only at the one or more portions thereof having the first additive exposed to the ultrasound.

Aspect 8a: The composite material, method, and/or device of any preceding Aspect, wherein the first additive increases one or more ultrasound-attenuation characteristic (optionally, an ultrasound absorption coefficient) of the composite material and/or of the one or more portions thereof (having the first additive) by at least 50% (optionally at least 75%, optionally at least 90%, optionally at least 100%, optionally at least 120%, optionally at least 140%, optionally at least 160%, optionally at least 180%, optionally at least 200%, optionally at least 220%, optionally at least 240%, optionally at least 260%, optionally at least 280%, optionally at least 300%, optionally at least 320%, optionally at least 340%) compared to that of the same one or more shape memory polymers free of said first additive (additive-free crosslinked shape memory polymer). Aspect 8b: The composite material, method, and/or device of any preceding Aspect, wherein the first additive increases an ultrasound absorption coefficient of the composite material and/or of the one or more portions thereof (having the first additive) by at least 50% (optionally at least 75%, optionally at least 90%, optionally at least 100%, optionally at least 120%, optionally at least 140%, optionally at least 160%, optionally at least 180%, optionally at least 200%, optionally at least 220%, optionally at least 240%, optionally at least 260%, optionally at least 280%, optionally at least 300%, optionally at least 320%, optionally at least 340%) compared to that of the same one or more shape memory polymers free of said first additive (additive-free crosslinked shape memory polymer). Aspect 8c: The composite material, method, and/or device of any preceding Aspect, wherein the first additive increases one or more ultrasound-attenuation characteristic (optionally, an ultrasound absorption coefficient) of the composite material and/or the one or more portions thereof (having the first additive) by at least 50% (optionally at least 75%, optionally at least 90%, optionally at least 100%, optionally at least 120%, optionally at least 140%, optionally at least 160%, optionally at least 180%, optionally at least 200%, optionally at least 220%, optionally at least 240%, optionally at least 260%, optionally at least 280%, optionally at least 300%, optionally at least 320%, optionally at least 340%) compared to that of same one or more shape memory polymers free of said first additive (additive-free crosslinked shape memory polymer) at ultrasound frequencies selected from the range of 400 kHz to 600 kHz (optionally at 500 kHz). Aspect 8d: The composite material, method, and/or device of any preceding Aspect, wherein the first additive increases an ultrasound absorption coefficient of the composite material and/or the one or more portions thereof (having the first additive) by at least 50% (optionally at least 75%, optionally at least 90%, optionally at least 100%, optionally at least 120%, optionally at least 140%, optionally at least 160%, optionally at least 180%, optionally at least 200%, optionally at least 220%, optionally at least 240%, optionally at least 260%, optionally at least 280%, optionally at least 300%, optionally at least 320%, optionally at least 340%) compared to that of the same one or more shape memory polymers free of said first additive (additive-free crosslinked shape memory polymer) at ultrasound frequencies selected from the range of 400 kHz to 600 kHz (optionally at 500 kHz).

Aspect 9a: The composite material, method, and/or device of any preceding Aspect, wherein the one or more ultrasound-absorption characteristic of the composite material or of the one or more portions of the composite material having the first additive is an ultrasound attenuation coefficient and/or an ultrasound absorption characteristic. Aspect 9b: The composite material, method, and/or device of any preceding Aspect, wherein the one or more ultrasound-absorption characteristics of the composite material or of the one or more portions of the composite material having the first additive is an ultrasound attenuation coefficient.

Aspect 10a: The composite material, method, and/or device of any preceding Aspect, wherein the composite material and/or at least the one or more portions thereof (having the first additive) are characterized by an ultrasound attenuation coefficient selected from the range of 0.05 dB/mm to 10 dB/mm. Aspect 10b: The composite material, method, and/or device of any preceding Aspect, wherein the composite material and/or the one or more portions thereof (having the first additive) is characterized by an ultrasound attenuation coefficient of at least 0.16 dB/mm (optionally at least 0.17 dB/mm, optionally at least 0.18 dB/mm, optionally at least 0.19 dB/mm, optionally at least 0.20 dB/mm, optionally at least 0.21 dB/mm, optionally at least 0.22 dB/mm, optionally at least 0.23 dB/mm, optionally at least 0.24 dB/mm, optionally at least 0.25 dB/mm, optionally at least 0.26 dB/mm, optionally at least 0.27 dB/mm, optionally at least 0.28 dB/mm, optionally at least 0.29 dB/mm, optionally at least 0.30 dB/mm, optionally at least 0.31 dB/mm, optionally at least 0.32 dB/mm, optionally at least 0.33 dB/mm, optionally at least 0.34 dB/mm, optionally at least 0.35 dB/mm, optionally at least 0.37 dB/mm, optionally at least 0.40 dB/mm, optionally at least 0.42 dB/mm, optionally at least 0.45 dB/mm, optionally at least 0.47 dB/mm, optionally at least 0.50 dB/mm, optionally at least 0.51 dB/mm, optionally at least 0.52 dB/mm, optionally at least 0.53 dB/mm, optionally at least 0.54 dB/mm, optionally at least 0.55 dB/mm, optionally at least 0.56 dB/mm, optionally at least 0.57 dB/mm, optionally at least 0.58 dB/mm, optionally at least 0.59 dB/mm, optionally at least 0.60 dB/mm, optionally at least 0.9 dB/mm, optionally at least 1.0 dB/mm, optionally at least 1.2 dB/mm, optionally at least 1.5 dB/mm, optionally at least 1.75 dB/mm, optionally at least 2.0 dB/mm, optionally at least 2.25 dB/mm, optionally at least 2.50 dB/mm, optionally at least 2.75 dB/mm, optionally at least 3.0 dB/mm, optionally at least 3.25 dB/mm, optionally at least 3.5 dB/mm, optionally at least 3.75 dB/mm) at ultrasound frequencies selected from the range of 400 kHz to 600 kHz (optionally at 500 kHz). Aspect 10c: The composite material, method, and/or device of any preceding Aspect, wherein the composite material and/or the one or more portions thereof (having the first additive) is characterized by an ultrasound attenuation coefficient of selected from the range of 0.15 dB/mm (optionally 0.5 dB/mm) to 10 dB/mm (optionally 5 dB/mm), wherein any value or range therebetween inclusively is explicitly contemplated. Aspect 10d: The composite material, method, and/or device of any preceding Aspect, wherein the composite material and/or the one or more portions thereof (having the first additive) is characterized by an ultrasound attenuation coefficient of selected from the range of 0.05 dB/mm (optionally 0.08 dB/mm) to 10 dB/mm, and wherein any value or range therebetween inclusively is explicitly contemplated.

Aspect 11a: The composite material, method, and/or device of any preceding Aspect, wherein T_{cm,trans} is selected from the range of 25° C. to 100° C. Aspect 11a: The composite material, method, and/or device of any preceding Aspect, wherein T_{cm,trans} is selected from the range of 25° C. (optionally 26° C., optionally 27° C., optionally 28° C., optionally 29° C., optionally 30° C., optionally 31° C., optionally 32° C., optionally 33° C., optionally 34° C., optionally 35° C., optionally 36° C., optionally 37° C., optionally 38° C., optionally 39° C., optionally 40° C.) to 150° C. (optionally 40° C., optionally 45° C., optionally 50° C., optionally 55° C., optionally 60° C., optionally 65° C., optionally 70° C., optionally 75° C., optionally 80° C., optionally 85° C., optionally 90° C., optionally 95° C., optionally 100° C., optionally 110° C., optionally 120° C., optionally 130° C., optionally 140° C.), wherein any value or range therebetween inclusively is explicitly contemplated.

Aspect 12: The composite material, method, and/or device of any preceding Aspect, wherein T_{cm,trans} is approximately the melt transition temperature (Tₘ) of the composite material.

Aspect 13: The composite material, method, and/or device of any preceding Aspect, wherein shape change is an expansion, a contraction, a twisting, an unraveling, a curling, an unfurling, an opening, a closing, a bending, an unbending, a folding, an unfolding, a straightening, a lengthening, a shortening, a redistribution or change in distribution of stress in the material, a redistribution or change in distribution of strain in the material, or any combination of these.

Aspect 14a The composite material, method, and/or device of any preceding Aspect, wherein the shape change occurs as a result of exposure of the composite material or the one or more portions thereof to ultrasound characterized by frequencies selected from the range of approximately 300 kHz (optionally within 20% thereof) to approximately 3 MHz (optionally within 20% thereof) and an energy intensity selected from the range of approximately 1 W/cm² (optionally within 20% thereof) to approximately 3 W/cm² (optionally within 20% thereof).

Aspect 14b: The composite material, method, and/or device of any preceding Aspect, wherein the shape change occurs and/or is complete within 300 seconds (optionally 260 seconds, optionally 240 seconds, optionally 200 seconds, optionally 180 seconds, optionally 150 seconds, optionally 120 seconds, optionally 100 seconds, optionally 60 seconds, optionally 45 seconds, optionally 30 seconds, optionally 15 seconds) of exposure of the composite material or the one or more portions thereof to ultrasound characterized by frequencies selected from the range of approximately 300 kHz to approximately 3 MHz and an energy intensity selected from the range of approximately 1 W/cm² to approximately 3 W/cm². Aspect 14c: The composite material, method, and/or device of any preceding Aspect, wherein the shape change occurs and/or is complete within 300 seconds (optionally 260 seconds, optionally 240 seconds, optionally 200 seconds, optionally 180 seconds, optionally 150 seconds, optionally 120 seconds, optionally 100 seconds, optionally 60 seconds, optionally 45 seconds, optionally 30 seconds, optionally 15 seconds) of exposure of the composite material or the one or more portions thereof to ultrasound.

Aspect 15a: The composite material, method, and/or device of any preceding Aspect, wherein the one or more portions having the first additive exhibit heating at a rate selected from the range of 0.1° C./s to 5° C./s, wherein any value or range therebetween inclusively is explicitly contemplated, with exposure to ultrasound characterized by frequencies selected from the range of approximately 300 kHz to approximately 3 MHz and an energy intensity selected from the range of approximately 1 W/cm² to approximately 3 W/cm². Aspect 15b: The composite material, method, and/or device of any preceding Aspect, wherein the one or more portions having the first additive exhibit an average heating rate selected from the range of 0.1° C./s to 5° C./s (where average heating rate corresponds to ΔT/timeₑₓₚₒₛᵤᵣₑ, where ΔT is the change in temperature from beginning of test to maximum temperature reached and timeₑₓₚₒₛᵤᵣₑ is the corresponding time of ultrasound exposure) with exposure to ultrasound characterized by frequencies selected from the range of approximately 300 kHz to approximately 3 MHz and an energy intensity selected from the range of approximately 1 W/cm² to approximately 3 W/cm². Aspect 15c: The composite material, method, and/or device of any preceding Aspect, wherein the one or more portions having the first additive exhibit an average heating rate selected from the range of 0.1° C./s (optionally 0.15° C./s, optionally 0.2 C/s, optionally 0.25 C/s, optionally 0.3 C/s) to 5° C./s (optionally 0.35 C/s, optionally 0.36 C/s, optionally 0.37 C/s, optionally 0.39 C/s, optionally 0.40 C/s, optionally 0.42 C/s, optionally 0.45 C/s, optionally 0.47 C/s, optionally 0.49° C./s) (where average heating rate corresponds to ΔT/timeₑₓₚₒₛᵤᵣₑ, where ΔT is the change in temperature from beginning of test to maximum temperature reached and timeₑₓₚₒₛᵤᵣₑ is the corresponding time of ultrasound exposure) with exposure to ultrasound characterized by frequencies selected from the range of approximately 300 kHz to approximately 3 MHz and an energy intensity selected from the range of approximately 1 W/cm² to approximately 3 W/cm². Aspect 15d: The composite material, method, and/or device of any preceding Aspect, wherein the one or more portions having the first additive exhibit an average heating rate selected from the range of 0.1° C./s (optionally 0.15° C./s, optionally 0.2 C/s, optionally 0.25 C/s, optionally 0.3 C/s) to 5° C./s (optionally 0.35 C/s, optionally 0.36 C/s, optionally 0.37 C/s, optionally 0.39 C/s, optionally 0.40 C/s, optionally 0.42 C/s, optionally 0.45 C/s, optionally 0.47 C/s, optionally 0.49° C./s) (where average heating rate corresponds to ΔT/timeₑₓₚₒₛᵤᵣₑ, where ΔT is the change in temperature from beginning of test to maximum temperature reached and timeₑₓₚₒₛᵤᵣₑ is the corresponding time of ultrasound exposure) with exposure to ultrasound.

Aspect 16a: The composite material, method, and/or device of any preceding Aspect, wherein the composite material is characterized by a density selected from the range of 0.01 to 22.5 g/cm³, and wherein any value or range therebetween inclusively is explicitly contemplated. Aspect 16a: The composite material, method, and/or device of any preceding Aspect, wherein the composite material is characterized by a Young's modulus selected from the range of 1.0 MPa to 1000 MPa at NTP, and wherein any value or range therebetween inclusively is explicitly contemplated, such as optionally 10 MPa to 100 MPa at NTP.

Aspect 17a: The composite material, method, and/or device of any preceding Aspect, wherein the first additive comprises a plurality of organic particles. Aspect 17b: The composite material, method, and/or device of any preceding Aspect, wherein the first additive comprises a plurality of inorganic particles. Aspect 17c: The composite material, method, and/or device of any preceding Aspect, wherein the first additive comprises a plurality of organic particles and a plurality of inorganic particles.

Aspect 18a: The composite material, method, and/or device of any preceding Aspect, wherein the first additive in the composite material is characterized by an ultrasound attenuation coefficient selected from the range of 0.05 dB/mm to 10 dB/mm. Aspect 18b: The composite material, method, and/or device of any preceding Aspect, wherein the first additive is an ultrasound-absorbing material characterized by an ultrasound attenuation coefficient of at least 0.16 dB/mm (optionally at least 0.17 dB/mm, optionally at least 0.18 dB/mm, optionally at least 0.19 dB/mm, optionally at least 0.20 dB/mm, optionally at least 0.21 dB/mm, optionally at least 0.22 dB/mm, optionally at least 0.23 dB/mm, optionally at least 0.24 dB/mm, optionally at least 0.25 dB/mm, optionally at least 0.26 dB/mm, optionally at least 0.27 dB/mm, optionally at least 0.28 dB/mm, optionally at least 0.29 dB/mm, optionally at least 0.30 dB/mm, optionally at least 0.31 dB/mm, optionally at least 0.32 dB/mm, optionally at least 0.33 dB/mm, optionally at least 0.34 dB/mm, optionally at least 0.35 dB/mm, optionally at least 0.37 dB/mm, optionally at least 0.40 dB/mm, optionally at least 0.42 dB/mm, optionally at least 0.45 dB/mm, optionally at least 0.47 dB/mm, optionally at least 0.50 dB/mm, optionally at least 0.51 dB/mm, optionally at least 0.52 dB/mm, optionally at least 0.53 dB/mm, optionally at least 0.54 dB/mm, optionally at least 0.55 dB/mm, optionally at least 0.56 dB/mm, optionally at least 0.57 dB/mm, optionally at least 0.58 dB/mm, optionally at least 0.59 dB/mm, optionally at least 0.60 dB/mm, optionally at least 0.65 dB/mm, optionally at least 0.70 dB/mm, optionally at least 0.75 dB/mm, optionally at least 0.80 dB/mm, optionally at least 0.85 dB/mm, optionally at least 0.9 dB/mm, optionally at least 1.0 dB/mm, optionally at least 1.2 dB/mm, optionally at least 1.5 dB/mm, optionally at least 1.75 dB/mm, optionally at least 2.0 dB/mm, optionally at least 2.25 dB/mm, optionally at least 2.50 dB/mm, optionally at least 2.75 dB/mm, optionally at least 3.0 dB/mm, optionally at least 3.25 dB/mm, optionally at least 3.5 dB/mm, optionally at least 3.75 dB/mm) at ultrasound frequencies selected from the range of 400 kHz to 600 kHz (optionally at 500 kHz). Aspect 18c: The composite material, method, and/or device of any preceding Aspect, wherein the first additive is an ultrasound-absorbing material characterized by an ultrasound attenuation coefficient of selected from the range of 0.15 dB/mm (optionally 0.5 dB/mm) to 10 dB/mm (optionally 5 dB/mm), wherein any value or range therebetween inclusively is explicitly contemplated.

Aspect 19: The composite material, method, and/or device of any preceding Aspect, wherein the first additive comprises a plurality of hollow particles (e.g., microspheres).

Aspect 20: The composite material, method, and/or device of any preceding Aspect, wherein the first additive comprises a plurality of non-hollow particles (e.g., microspheres).

Aspect 21: The composite material, method, and/or device of any preceding Aspect, wherein the first additive comprises a plurality of hollow glass beads (e.g., microspheres; e.g., glass bubbles), non-hollow glass beads (e.g., microspheres), or any combination thereof.

Aspect 22: The composite material, method, and/or device of any preceding Aspect, wherein the first additive comprises a plurality of salt particles, a plurality of metal oxide particles, a plurality of metal particles, a plurality of organic particles, or any combination thereof.

Aspect 23a: The composite material, method, and/or device of any preceding Aspect, wherein the first additive comprises a plurality of metal chloride particles, iron oxide particles, silica particles, silica gel particles, glass beads (e.g., glass particles, glass bubbles, or glass microspheres), metal particles (e.g., gold, platinum, iridium, silver, tungsten, or another metal having similar high density), or any combination thereof. Aspect 23b: The composite material, method, and/or device of any preceding Aspect, wherein the first additive comprises a plurality of metal chloride particles, iron oxide particles, silica particles, silica gel particles, glass beads (e.g., glass particles, glass bubbles, or glass microspheres), metal particles (e.g., gold, platinum, iridium, silver, tungsten, or another metal having similar high density, or alloys thereof, or alloys therewith; e.g., high density metal alloys), poly(tetrafluoroethylene)(PTFE) particles, or any combination thereof.

Aspect 24a: The composite material, method, and/or device of any preceding Aspect, wherein the first additive comprises a plurality of hollow glass microspheres or particles and/or non-hollow glass microspheres or particles characterized by a median diameter selected from the range of approximately 15 μm (optionally 16 μm, optionally 17 μm, optionally 18 μm, optionally 19 μm, optionally 20 μm, optionally 21 μm, optionally 22 μm, optionally 23 μm, optionally 24 μm, optionally 25 μm, optionally 30 μm, optionally 35 μm) to approximately 1000 μm (optionally 36 μm, optionally 40 μm, optionally 45 μm, optionally 50 μm, optionally 55 μm, optionally 60 μm, optionally 65 μm, optionally 70 μm, optionally 75 μm, optionally 80 μm, optionally 85 μm, optionally 90 μm, optionally 95 μm, optionally 100 μm, optionally 200 μm, optionally 300 μm, optionally 400 μm, optionally 500 μm, optionally 600 μm, optionally 700 μm, optionally 800 μm, optionally 900 μm). Aspect 24b: The composite material, method, and/or device of any preceding Aspect, wherein the first additive comprises a plurality of hollow glass microspheres or particles and/or non-hollow glass microspheres or particles characterized by an average diameter selected from the range of approximately 15 μm (optionally 16 μm, optionally 17 μm, optionally 18 μm, optionally 19 μm, optionally 20 μm, optionally 21 μm, optionally 22 μm, optionally 23 μm, optionally 24 μm, optionally 25 μm, optionally 30 μm, optionally 35 μm) to approximately 1000 μm (optionally 36 μm, optionally 40 μm, optionally 45 μm, optionally 50 μm, optionally 55 μm, optionally 60 μm, optionally 65 μm, optionally 70 μm, optionally 75 μm, optionally 80 μm, optionally 85 μm, optionally 90 μm, optionally 95 μm, optionally 100 μm, optionally 200 μm, optionally 300 μm, optionally 400 μm, optionally 500 μm, optionally 600 μm, optionally 700 μm, optionally 800 μm, optionally 900 μm), wherein any value or range therebetween inclusively is explicitly contemplated. Aspect 24c: The composite material, method, and/or device of any preceding Aspect, wherein the first additive comprises a plurality of hollow particles and/or non-hollow particles characterized by a median diameter selected from the range of approximately 15 μm (optionally 16 μm, optionally 17 μm, optionally 18 μm, optionally 19 μm, optionally 20 μm, optionally 21 μm, optionally 22 μm, optionally 23 μm, optionally 24 μm, optionally 25 μm, optionally 30 μm, optionally 35 μm) to approximately 1000 μm (optionally 36 μm, optionally 40 μm, optionally 45 μm, optionally 50 μm, optionally 55 μm, optionally 60 μm, optionally 65 μm, optionally 70 μm, optionally 75 μm, optionally 80 μm, optionally 85 μm, optionally 90 μm, optionally 95 μm, optionally 100 μm, optionally 200 μm, optionally 300 μm, optionally 400 μm, optionally 500 μm, optionally 600 μm, optionally 700 μm, optionally 800 μm, optionally 900 μm), wherein any value or range therebetween inclusively is explicitly contemplated.

Aspect 25: The composite material, method, and/or device of any preceding Aspect, wherein the first additive comprises a plurality of hollow microspheres characterized by a median internal cavity diameter selected from the range of 1 μm (optionally 2 μm, optionally 3 μm, optionally 4 μm, optionally 5 μm, optionally 6 μm, optionally 7 μm, optionally 8 μm, optionally 9 μm, optionally 10 μm, optionally 11 μm, optionally 12 μm, optionally 13 μm, optionally 14 μm, optionally 15 μm, optionally 20 μm) to 990 μm (optionally 6 μm, optionally 7 μm, optionally 8 μm, optionally 9 μm, optionally 10 μm, optionally 11 μm, optionally 12 μm, optionally 13 μm, optionally 14 μm, optionally 15 μm, optionally 20 μm, optionally 25 μm, optionally 30 μm, optionally 50 μm, optionally 100 μm, optionally 150 μm, optionally 200 μm) and/or an average wall thickness selected from the range of 0.2 μm (optionally 0.3 μm, optionally 0.4 μm, optionally 0.5 μm, optionally 0.6 μm, optionally 0.7 μm, optionally 0.8 μm, optionally 0.9 μm, optionally 1 μm) to 5 μm (optionally 1.2 μm, optionally 1.3 μm, optionally 1.4 μm, optionally 1.5 μm, optionally 2 μm, optionally 3 μm, optionally 4 μm, optionally 4.5 μm), wherein any value or range therebetween inclusively is explicitly contemplated.

Aspect 26a: The composite material, method, and/or device of any preceding Aspect, wherein a concentration of the first additive in the one or more shape memory polymers is selected from the range of 0.4 wt. % (optionally 0.5 wt. %, optionally 0.6 wt. %, optionally 0.7 wt. %, optionally 0.8 wt. %, optionally 0.9 wt. %, optionally 1.0 wt. %, optionally 1.1 wt. %, optionally 1.2 wt. %, optionally 1.5 wt. %, optionally 1.7 wt. %, optionally 2.0 wt. %, optionally 2.2 wt. %, optionally 2.5 wt. %, optionally 2.7 wt. %, optionally 3.0 wt. %, optionally 3.2 wt. %, optionally 3.5 wt. %, optionally 3.7 wt. %, optionally 4.0 wt. %, optionally 4.2 wt. %, optionally 4.5 wt. %, optionally 4.7 wt. %, optionally 5.0 wt. %) to 50 wt. % (optionally 5 wt. %, optionally 6 wt. %, optionally 7 wt. %, optionally 8 wt. %, optionally 9 wt. %, optionally 10 wt. %, optionally 11 wt. %, optionally 12 wt. %, optionally 13 wt. %, optionally 14 wt. %, optionally 15 wt. %, optionally 20 wt. %, optionally 25 wt. %, optionally 30 wt. %, optionally 35 wt. %, optionally 40 wt. %, optionally 45 wt. %), wherein any value or range therebetween inclusively is explicitly contemplated, with respect to weight of the one or more polymers in the composite material. Aspect 26b: The composite material, method, and/or device of any preceding Aspect, wherein the first additive comprises a plurality of metal oxide particles (e.g., iron oxide particles); and wherein a concentration of the first additive in the one or more shape memory polymers is selected from the range of approximately 1 wt. % (optionally 1.0 wt. %, optionally 1.1 wt. %, optionally 1.2 wt. %, optionally 1.5 wt. %, optionally 1.7 wt. %, optionally 2.0 wt. %, optionally 2.2 wt. %, optionally 2.5 wt. %, optionally 2.7 wt. %, optionally 3.0 wt. %, optionally 3.2 wt. %, optionally 3.5 wt. %, optionally 3.7 wt. %, optionally 4.0 wt. %, optionally 4.2 wt. %, optionally 4.5 wt. %, optionally 4.7 wt. %, optionally 5.0 wt. %) to 30 wt. % (optionally 5 wt. %, optionally 6 wt. %, optionally 7 wt. %, optionally 8 wt. %, optionally 9 wt. %, optionally 10 wt. %, optionally 11 wt. %, optionally 12 wt. %, optionally 13 wt. %, optionally 14 wt. %, optionally 15 wt. %), wherein any value or range therebetween inclusively is explicitly contemplated, with respect to weight of the one or more polymers in the composite material. Aspect 26c: The composite material, method, and/or device of any preceding Aspect, wherein the first additive comprises a plurality of hollow and/or non-hollow glass microspheres (e.g., K25 hollow glass microspheres; e.g., iM30K hollow glass microspheres); and wherein a concentration of the first additive in the one or more shape memory polymers is selected from the range of approximately 0.4 wt. % (optionally 0.5 wt. %, optionally 0.6 wt. %, optionally 0.7 wt. %, optionally 0.8 wt. %, optionally 0.9 wt. %, optionally 1.0 wt. %, optionally 1.1 wt. %, optionally 1.2 wt. %, optionally 1.5 wt. %) to 50 wt. % (optionally 5 wt. %, optionally 6 wt. %, optionally 7 wt. %, optionally 8 wt. %, optionally 9 wt. %, optionally 10 wt. %), wherein any value or range therebetween inclusively is explicitly contemplated, with respect to weight of the one or more polymers in the composite material.

Aspect 27a: The composite material, method, and/or device of any preceding Aspect, wherein the first additive is biologically inert and/or is substantially insoluble in a biological fluid under physiological or in-vivo conditions. Aspect 27b: The composite material, method, and/or device of any preceding Aspect, wherein the first additive is substantially insoluble in saline fluid over a time period selected from the range of 1 day to at least 6 months.

Aspect 28: The composite material, method, and/or device of any preceding Aspect, wherein the first additive or the plurality of particles thereof is characterized by a density selected from the range of 0.01 g/cm³ (optionally 0.02 g/cm³, optionally 0.03 g/cm³, optionally 0.05 g/cm³, optionally 0.07 g/cm³, optionally 0.09 g/cm³, optionally 0.1 g/cm³, optionally 1.2 g/cm³, optionally 1.5 g/cm³, optionally 1.7 g/cm³, optionally 2.0 g/cm³, optionally 2.5 g/cm³) to 22.5 g/cm³ (optionally 0.8 g/cm³, optionally 1.0 g/cm³, optionally 1.5 g/cm³, optionally 2.0 g/cm³, optionally 2.5 g/cm³, optionally 3.0 g/cm³, optionally 3.5 g/cm³, optionally 4.0 g/cm³, optionally 4.5 g/cm³, optionally 5.0 g/cm³, optionally 5.2 g/cm³, optionally 5.5 g/cm³, optionally 6.0 g/cm³, optionally 6.5 g/cm³), optionally 0.1 g/cm³ to 5.2 g/cm³, wherein any value or range therebetween inclusively is explicitly contemplated.

Aspect 29: The composite material, method, and/or device of any preceding Aspect, wherein the first additive or the plurality of particles thereof is characterized by a heat capacity selected from the range of 0.10 JC⁻¹g⁻¹ (optionally 0.11 JC⁻¹g⁻¹, optionally 0.15 JC⁻¹g⁻¹, optionally 0.20 JC⁻¹g⁻¹, optionally 0.25 JC⁻¹g⁻¹, optionally 0.30 JC⁻¹g⁻¹, optionally 0.5 JC⁻¹g⁻¹, optionally 0.65 JC⁻¹g⁻¹, optionally 0.7 JC⁻¹g⁻¹, optionally 0.75 JC⁻¹g⁻¹, optionally 0.8 JC⁻¹g⁻¹, optionally 0.9 JC⁻¹g⁻¹, optionally 1.0 JC⁻¹g⁻¹, optionally 1.5 JC⁻¹g⁻¹) to 4.5 JC⁻¹g⁻¹ (optionally 1.0 JC⁻¹g⁻¹, optionally 1.5 JC⁻¹g⁻¹, optionally 2.0 JC⁻¹g⁻¹, optionally 2.5 JC⁻¹g⁻¹, optionally 3.0 JC⁻¹g⁻¹, optionally 3.5 JC⁻¹g⁻¹, optionally 4.0 JC⁻¹g⁻¹, optionally 4.2 JC⁻¹g⁻¹, optionally 4.5 JC⁻¹g⁻¹, optionally 5.0 JC⁻¹g⁻¹), optionally 0.7 JC⁻¹g⁻¹ to optionally 1.0 JC⁻¹g⁻¹, wherein any value or range therebetween inclusively is explicitly contemplated.

Aspect 30: The composite material, method, and/or device of any preceding Aspect, wherein the first additive or the plurality of particles thereof is characterized by a thermal conductivity selected from the range of 0.02 Wm⁻¹K⁻¹ (optionally 0.03 Wm⁻¹K⁻¹, optionally 0.04 Wm⁻¹K⁻¹, optionally 0.05 Wm⁻¹K⁻¹, optionally 0.08 Wm⁻¹K⁻¹, optionally 0.10 Wm⁻¹K⁻¹, optionally 0.15 Wm⁻¹K⁻¹, optionally 0.20 Wm⁻¹K⁻¹, optionally 0.25 Wm⁻¹K⁻¹, optionally 0.5 Wm⁻¹K⁻¹, optionally 0.75 Wm⁻¹K⁻¹, optionally 1.0 Wm⁻¹K⁻¹, optionally 1.25 Wm⁻¹K⁻¹, optionally 1.5 Wm⁻¹K⁻¹) to 500.00 Wm⁻¹K⁻¹ (optionally 2 Wm⁻¹K⁻¹, optionally 3 Wm⁻¹K⁻¹, optionally 5 Wm⁻¹K⁻¹, optionally 6 Wm⁻¹K⁻¹, optionally 9 Wm⁻¹K⁻¹, optionally 10 Wm⁻¹K⁻¹, optionally 15 Wm⁻¹K⁻¹, optionally 50 Wm⁻¹K⁻¹, optionally 100 Wm⁻¹K⁻¹, optionally 200 Wm⁻¹K⁻¹, optionally 300 Wm⁻¹K⁻¹, optionally 400 Wm⁻¹K⁻¹, optionally 428 Wm⁻¹K⁻¹, optionally 500 Wm⁻¹K⁻¹), optionally 0.04 Wm⁻¹K⁻¹ to 6.0 Wm⁻¹K⁻¹, wherein any value or range therebetween inclusively is explicitly contemplated.

Aspect 31: The composite material, method, and/or device of any preceding Aspect, wherein the first additive or the plurality of particles thereof is characterized by a density selected from the range of 0.1 to 0.6 g/cm³, wherein any value or range therebetween inclusively is explicitly contemplated, such as optionally 0.25 to 0.6 g/cm³.

Aspect 32: The composite material, method, and/or device of any preceding Aspect, wherein the first additive or the plurality of particles thereof is characterized by a thermal conductivity selected from the range of 0.04 to 0.20 Wm⁻¹K⁻¹, wherein any value or range therebetween inclusively is explicitly contemplated.

Aspect 33a: The composite material, method, and/or device of any preceding Aspect, wherein the first additive comprises or is (optionally, is) a plurality of particles having characteristic sizes selected from the range of 0.030 to 1000 μm, wherein any value or range therebetween inclusively is explicitly contemplated, such as optionally 15 μm to 55 μm. Aspect 33b: The composite material, method, and/or device of any preceding Aspect, wherein the first additive comprises or is (optionally, is) a plurality of particles characterized by a median size selected from the range of 0.030 to 1000 μm, wherein any value or range therebetween inclusively is explicitly contemplated, such as optionally 15 μm to 55 μm. Aspect 33c: The composite material, method, and/or device of any preceding Aspect, wherein the first additive comprises or is (optionally, is) a plurality of particles characterized by an average size selected from the range of 0.030 μm to 1000 μm, wherein any value or range therebetween inclusively is explicitly contemplated, such as optionally 15 μm to 55 μm. Aspect 33d: The composite material, method, and/or device of any preceding Aspect, wherein the first additive comprises a plurality of particles having a median characteristic size selected from the range of 0.030 to 1000 μm, wherein any value or range therebetween inclusively is explicitly contemplated, such as optionally 15 μm to 55 μm.

Aspect 34: The composite material, method, and/or device of any preceding Aspect, wherein one or more additive-free crosslinked shape memory polymers equivalent to the one or more shape memory polymers of the composite material are characterized by a polymer-only transition temperature (T_{pol,trans}); and wherein T_{cm,trans} deviates from T_{pol,trans} by no more than 5° C. (optionally no more than 4° C., optionally no more than 3° C., optionally no more than 2° C., optionally no more than 1.5° C., optionally no more than 1° C.) and/or no more than 10% (optionally no more than 8%, optionally no more than 5%). For example, Tₘ varies by no more than 1.5° C. with vs. without additives where the additives are 5 wt. % HGMs or SGMs (MK-1-39, 8 examples, DBzP as crosslinker). For example, Tₘ varies by no more than 2.0° C. with vs. without additives, where additives are HGMs or SGMs (MK-1-21, 12 examples, DCP as crosslinker, varying wt. % 0.5-5.0). With Fe₃O₄, large variations in Tₘ were seen at 5.0 and 15.0 wt. %. For example, Tₘ varies by no more than 1.9° C. with vs. without additives, where additives are NaCl, silica gel, K25 HGMs, Fe3O4 NPs, are used at 1.0 wt. % (MK1-7, 8 examples, di-COE as crosslinker).

Aspect 35: The composite material, method, and/or device of any preceding Aspect, wherein the one or more shape memory polymers is characterized by an upper limit of crosslinking temperature of at least 65° C. (optionally at least 70° C., optionally at least 75° C., optionally at least 80° C., optionally at least 85° C., optionally at least 90° C., optionally at least 95° C., optionally at least 100° C., optionally at least 105° C., optionally at least 110° C., optionally at least 115° C., optionally at least 120° C., optionally at least 125° C., optionally at least 130° C.).

Aspect 36: The composite material, method, and/or device of any preceding Aspect, wherein the one or more shape memory polymers comprise crosslinking moieties derived from a crosslinking precursor is one or more organic peroxide compounds having a 10-hour half-life temperature (HLT) at least 10° C. greater (optionally at least 5° C. greater, optionally at least 12° C. greater, optionally at least 15° C. greater) than a melt temperature (Tₘ) and/or the T_{pol,trans} of the one or more shape memory polymers.

Aspect 37a: The composite material, method, and/or device of any preceding Aspect, wherein the one or more shape memory polymers comprise a crosslinking moieties derived from a crosslinking precursor selected from the group consisting of: a di(4-cyclooctenol) succinate, dicumyl peroxide (DCP), dibenzoyl peroxide (DBzP), di(tert-butyl) peroxide, and any combination thereof. Aspect 37b: The composite material, method, and/or device of any preceding Aspect, wherein the one or more shape memory polymers comprise a crosslinking moieties derived from a crosslinking precursor selected from the group consisting of: a di(4-cyclooctenol) succinate, dicumyl peroxide (DCP), dibenzoyl peroxide (DBzP), di(tert-butyl) peroxide, any derivative thereof, any analogue thereof, and any combination thereof.

Aspect 38a: The composite material, method, and/or device of any preceding Aspect, wherein one or more shape memory polymers comprise a poly(cyclooctene), a polycaprolactone, a poly(lactic acid), a poly(lactic-co-glycolic acid), a polyethylene, a polypropylene, a thermoplastic polyurethane (TPU), or any combination thereof. Aspect 38b: The composite material, method, and/or device of any preceding Aspect, wherein the one or more shape memory polymers comprise a poly(cyclooctene), a polycaprolactone, or any combination thereof. Aspect 38c: The composite material, method, and/or device of any preceding Aspect, wherein one or more shape memory polymers comprise a poly(cyclooctene), a polycaprolactone, a poly(lactic acid), a poly(lactic-co-glycolic acid), a polyethylene, a polypropylene, a thermoplastic polyurethane (TPU), any derivative thereof, any analogue thereof, or any combination thereof.

Aspect 39: The composite material, method, and/or device of any preceding Aspect, comprising a second additive being different from the first additive.

Aspect 40a: The composite material, method, and/or device of Aspect 39, wherein the second additive comprises a plurality of inorganic particles, a plurality of hollow particles, a plurality of particles characterized by an ultrasound attenuation coefficient selected from the range of 0.05 dB/mm to 10 dB/mm, or any combination thereof. Aspect 40b: The composite material, method, and/or device of Aspect 39, wherein the second additive is according to any of the preceding Aspects but is different from the first additive.

Aspect 41: The composite material, method, and/or device of any preceding Aspect, wherein the temporary shape is compressed with respect to the permanent shape and wherein the shape change comprises expansion.

Aspect 42: A device comprising the composite material of any preceding Aspect.

Aspect 43: The device of Aspect 42 or 1g, wherein the composite material is an ultrasound-activated actuator or switch of the device.

Aspect 44: The device of Aspect 42, 43, or 1g being a medical device.

Aspect 45: The device of Aspect 42, 43, 44, or 1g being a medical tool, a medical accessory, or medical component for a therapeutic treatment of a living subject or for a surgical procedure on a living subject.

Aspect 46: A method of using the composite material of any preceding Aspect, the method comprising:
directing one or more focused ultrasound beams at the one or more portions of the composite material;
thereby, heating the one or more portions to a temperature approximately equal to or greater than the T_{cm,trans}; and
thereby, causing the composite material to undergo the shape change at the one or more portions thereof.

Aspect 47: The method of Aspect 46 or 1d, wherein the step of directing comprises exposing each of the one or more portions to the one or more focused ultrasound beams for a consecutive/uninterrupted time being less than 5 minutes.

Aspect 48: The method of Aspect 46, 47, or 1d, wherein the step of directing comprises controlling and varying an exposure time, power, and/or exposure area of the one or more focused ultrasound beams.

Aspect 49: The method of any of Aspects 46-48 or 1d, wherein the step of directing comprises exposing the one or more portions to different focused ultrasound beams characterized by different exposure time, power, and/or exposure area.

Aspect 50: The method of any of Aspects 46-49 or 1d comprising actuating an actuator or switch of a device; wherein the actuator or switch comprises the composite material; and wherein the shape change causes the actuating.

Aspect 51a: The method of any of Aspects 46-50 or 1d comprising setting the temporary shape of the composite material at a temperature equal to or greater than T_{cm,trans} and less than a crosslinking temperature (T_{cm,crosslink}) of the composite material. Aspect 51b: The method of any of Aspects 46-50 or 1d comprising setting the temporary shape of the composite material at a temperature equal to or greater than T_{cm,trans} and maintaining said temperature as the composite material is cooled to below T_{cm,trans}. Aspect 51c: The method of any of Aspects 46-50 or 1 d comprising setting the temporary shape of the composite material at a temperature equal to or greater than T_{cm,trans}.

Aspect 52: A method of making the composite material of any of the preceding Aspects, the method comprising:
polymerizing a monomer to form a first polymer;
crosslinking the first polymer in the presence of a crosslinking precursor and the first additive at a temperature approximately (e.g., within 20%) equal to or greater than a crosslinking temperature (T_{cm,crosslink}) (optionally at a temperature greater than or equal to 65° C., optionally a temperature greater than or equal to 75° C., a temperature greater than or equal to 85° C., a temperature greater than or equal to 90° C., a temperature greater than or equal to 95° C., a temperature greater than or equal to 100° C., a temperature greater than or equal to 105° C., a temperature greater than or equal to 110° C., a temperature greater than or equal to 115° C.) to form the composite material having the crosslinked shape memory polymer and the first additive.

Aspect 53: The method of Aspect 52 or 1e, wherein the first additive is provided with the monomer and the step of polymerizing is performed in the presence of the first additive such that the first polymer comprises the first additive.

Aspect 54: The method of Aspect 52 or 1e, wherein the step of polymerization is performed in absence of the first additive and the first additive is provided to the step of crosslinking.

Aspect 55: The method of any of Aspects 52-54 or 1e, wherein the step of crosslinking is performed separately after the step of polymerizing.

Aspect 56: The method of any of Aspects 52-55 or 1e, wherein the steps of polymerizing and crosslinking are performed substantially concurrently as one step.

Aspect 57a: The method of any of Aspects 52-56 or 1e, wherein the crosslinking precursor is selected from the group consisting of: a di(4-cyclooctenol) succinate, dicumyl peroxide (DCP), dibenzoyl peroxide (DBzP), di(tert-butyl) peroxide, and any combination thereof. Aspect 57b: The method of any of Aspects 52-56 or 1e, wherein the crosslinking precursor is selected from the group consisting of: a di(4-cyclooctenol) succinate, dicumyl peroxide (DCP), dibenzoyl peroxide (DBzP), di(tert-butyl) peroxide, any derivative thereof, any analogue thereof, and any combination thereof.

Aspect 58a: The method of any of Aspects 52-57 or 1e, wherein the monomer is selected from the group consisting of: cyclooctene, cyclopentene, cycloheptene, butadiene, and any combination thereof. Aspect 58b: The method of any of Aspects 52-57 or 1e, wherein the monomer is selected from the group consisting of: cyclooctene, cyclopentene, cycloheptene, butadiene, any derivative thereof, any analogue thereof, and any combination thereof.

Aspect 59: The method of any of Aspects 52-58 or 1e, comprising selecting the first additive and a concentration thereof to tune the T_{cm,trans} and one or more ultrasound-absorption characteristics of the resulting composite material.

Aspect 60: The method of any of Aspects 52-59 or 1e, wherein the step of crosslinking comprises setting the permanent shape of the composite material at a temperature equal to or greater than the crosslinking temperature (T_{cm,crosslink}) for a crosslinking time period.

Aspect 61: The method of Aspect 60 or 1e, wherein the step of setting the permanent shape comprises confining the composite material, molding the composite material, or otherwise applying a mechanical force to the composite material while the composite material or at least a portion thereof is at a temperature equal to or greater than the crosslinking temperature (T_{cm,crosslink}).

Aspect 62: The method of any of Aspects 60-61 or 1e, wherein an upper limit of crosslinking temperature of the composite material is at least 65° C.

Aspect 63a: The method of any of Aspects 52-62 or 1e comprising setting the temporary shape of the composite material at a temperature within 35° C. of T_{cm,trans} or a temperature approximately equal to or greater than T_{cm,trans} and less than a crosslinking temperature (T_{cm,crosslink}) or an upper limit of crosslinking temperature of the composite material. Aspect 63b: The method of any of Aspects 52-62 comprising setting the temporary shape of the composite material at a temperature equal to or greater than T_{cm,trans} and maintaining said temperature as the composite material is cooled to below T_{cm,trans}. Aspect 63c: The method of any of Aspects 52-62 comprising setting the temporary shape of the composite material at a temperature equal to or greater than T_{cm,trans}.

Aspect 64: The method of Aspect 63 or 1e, wherein the step of setting the temporary shape comprises confining the composite material, molding the composite material, or otherwise applying a mechanical force to the composite material while is composite material or at least a portion thereof is at a temperature equal to or greater than T_{cm,trans}.

Aspect 65: A method of making the device of any of the preceding claims, the method comprising:
attaching, providing, or inserting the composite material of any of the preceding Aspects to or into the device.

Aspect 66: The method of Aspect 65 or 1f, comprising setting the temporary shape of the composite material at a temperature within 35° C. of T_{cm,trans} or a temperature approximately equal to or greater than T_{cm,trans} and less than a crosslinking temperature (T_{cm,crosslink}) or an upper limit of crosslinking temperature of the composite material. Aspect 66b: The method of any of Aspects 65 or 1f comprising setting the temporary shape of the composite material at a temperature equal to or greater than T_{cm,trans} and maintaining said temperature as the composite material is cooled to below T_{cm,trans}. Aspect 66c: The method of any of Aspects 65 or 1f comprising setting the temporary shape of the composite material at a temperature equal to or greater than T_{cm,trans}.

Aspect 67: The method of Aspect 66 or 1f, wherein the step of setting the temporary shape comprises confining the composite material, molding the composite material, or otherwise applying a mechanical force to the composite material while is composite material or at least a portion thereof is at a temperature equal to or greater than T_{cm,trans}.

Aspect 68: The method of any of Aspects 65-67 or 1f, wherein the step of setting the temporary shape is performed prior to attaching, providing, or inserting the composite material to or into the device.

Aspect 69: A method of making the device of any of the preceding claims, the method comprising: shaping or forming the composite material of any of the preceding Aspects thereby forming the device, the device being substantially formed of the composite material.

Aspect 70a: The composite material, method, and/or device of any preceding Aspect having one shape memory polymer. Aspect 70b: The composite material, method, and/or device of any preceding Aspect having two or more shape memory polymers.

Aspect 71: The composite material, method, and/or device of any preceding Aspect, wherein the shape change is from the temporary shape of the composite material an intermediate shape, the intermediate shape being a shape at some point between the temporary shape and the original permanent shape but before or without obtaining the original permanent shape.

Aspect 72: The method of Aspect 52-64 or 1e comprising providing the first additive at only one or more portions of the shape memory polymer(s) such that the first additive is non-uniformly or non-homogeneously provided in the shape memory polymer(s).

Aspect 73a: The composite material, method, and/or device of any preceding Aspect, wherein the first additive is non-uniformly or non-homogeneously present in the shape memory polymer(s). Aspect 73b: The composite material, method, and/or device of any preceding Aspect, wherein the first additive is uniformly or homogeneously present in the shape memory polymer(s).

Aspect 74: A composite material comprising:
one or more shape memory polymers; and
a first additive provided in the one or more shape memory polymers; wherein:
(a) the first additive comprises or is (optionally, is) a plurality of inorganic particles, (b) the first additive increases an ultrasound attenuation coefficient of the composite material (or at least of one or more portions thereof having the first additive) compared to that of the same one or more shape memory polymers free of said first additive; and/or (c) the first additive comprises or is (optionally, is) a plurality of hollow particles;
the composite material is characterized by a composite transition temperature (T_{cm,trans});
the first additive is provided at least at one or more portions of the composite material; and
the composite material or the one or more portions thereof undergo a shape change from a temporary shape to a permanent shape when the composite material or said one or more portions thereof are heated to within 35° C. of T_{cm,trans} or a temperature approximately equal to or greater than T_{cm,trans}.

Aspect 75: The composite material of Aspect 74, wherein the first additive is provided throughout the internal volume of the one or more shape memory polymers.

Aspect 76: The composite material of Aspect 74, wherein the one or more portions of the composite material having the first additive are heated when said one or more portions of the composite material are exposed to ultrasound.

Aspect 77: The composite material of claim Aspect 76, wherein the first additive absorbs the ultrasound; and wherein the first additive is heated by its absorption of ultrasound and/or wherein heat is created by friction between the first additive and the one or more shape memory polymers when the first additive absorbs the ultrasound.

Aspect 78: The composite material of Aspect 74, wherein the composite material undergoes the shape change only at the one or more portions thereof having the first additive exposed to the ultrasound.

Aspect 79: The composite material of Aspect 74, wherein the first additive increases the ultrasound attenuation coefficient of the composite material or of the one or more portions thereof having the first additive by at least 100% compared to that of the same one or more shape memory polymers free of said first additive.

Aspect 80: The composite material of Aspect 74, wherein the composite material or at least the one or more portions thereof are characterized by an ultrasound attenuation coefficient selected from the range of 0.05 dB/mm to 10 dB/mm.

Aspect 81: The composite material of Aspect 74, wherein T_{cm,trans} is selected from the range of 25 to 100° C.

Aspect 82: The composite material of Aspect 81, wherein T_{cm,trans} is the melt transition temperature (Tₘ) of the composite material.

Aspect 83: The composite material of Aspect 74, wherein shape change is an expansion, a contraction, a twisting, an unraveling, a curling, an unfurling, an opening, a closing, a bending, an unbending, a folding, an unfolding, a straightening, a lengthening, a shortening, a redistribution or change in distribution of stress in the material, a redistribution or change in distribution of strain in the material, or any combination of these.

Aspect 84: The composite material of Aspect 74, wherein the shape change occurs as a result of exposure of the composite material or the one or more portions thereof to ultrasound characterized by frequencies selected from the range of approximately 300 kHz to approximately 3 MHz and an energy intensity selected from the range of approximately 1 W/cm² to approximately 3 W/cm².

Aspect 85: The composite material of Aspect 74, wherein the one or more portions having the first additive exhibit heating at a rate of 0.1 C/s to 5 C/s with exposure to ultrasound characterized by frequencies selected from the range of approximately 300 kHz to approximately 3 MHz and an energy intensity selected from the range of approximately 1 W/cm² to approximately 3 W/cm².

Aspect 86: The composite material of Aspect 74, wherein the composite material is characterized by Young's modulus selected from the range of 1.0 MPa to 1000 MPa at NTP.

Aspect 87: The composite material of Aspect 74, wherein the first additive comprises a plurality of organic particles.

Aspect 88: The composite material of Aspect 74, wherein the first additive in the composite material is characterized by an ultrasound attenuation coefficient selected from the range of 0.05 dB/mm to 10 dB/mm.

Aspect 89: The composite material of Aspect 74, wherein the first additive comprises a plurality of hollow particles.

Aspect 90: The composite material of Aspect 74, wherein the first additive comprises a plurality of hollow glass beads, non-hollow glass beads, or any combination thereof.

Aspect 91: The composite material of Aspect 74, wherein the first additive comprises a plurality of salt particles, a plurality of metal oxide particles, a plurality of metal particles, a plurality of organic particles, or any combination thereof.

Aspect 92 composite material of Aspect 74, wherein the first additive comprises a plurality of hollow glass microspheres and/or non-hollow glass microspheres characterized by a median diameter selected from the range of 15 to 1000 μm.

Aspect 93: The composite material of Aspect 92, wherein the first additive comprises a plurality of hollow microspheres characterized by a median internal cavity diameter selected from the range of 1 to 100 μm and/or an average wall thickness selected from the range of 0.8 to 1.2 μm.

Aspect 94: The composite material of Aspect 74, wherein a concentration of the first additive in the one or more shape memory polymers is selected from the range of 0.5 wt. % to 50 wt. % with respect to weight of the one or more polymers.

Aspect 95: The composite material of Aspect 74, wherein the first additive is biologically inert and/or is substantially insoluble in a biological fluid under physiological conditions.

Aspect 96: The composite material of Aspect 74, wherein the first additive is characterized by a density selected from the range of 0.01 to 22.5 g/cm³, a heat capacity selected from the range of 0.11 to 4.2 JC⁻¹g⁻¹, and a thermal conductivity selected from the range of 0.02 to 428.00 Wm⁻¹K⁻¹; and wherein the first additive comprises a plurality of particles having a median characteristic size selected from the range of 0.030 to 1000 μm.

Aspect 97: The composite material of Aspect 74, wherein one or more additive-free crosslinked shape memory polymers equivalent to the one or more shape memory polymers of the composite material are characterized by a polymer-only transition temperature (T_{pol,trans}); and wherein T_{cm,trans} deviates from T_{pol,trans} by no more than 5° C. and/or 10%.

Aspect 98: The composite material of Aspect 74, wherein the one or more shape memory polymers comprise crosslinking moieties derived from a crosslinking precursor selected from the group consisting of an organic peroxide having a 10-hour half-life temperature (HLT) at least 10° C. greater than a melt temperature (Tₘ) of the one or more shape memory polymers.

Aspect 99: The composite material of Aspect 74, wherein the one or more shape memory polymers comprise crosslinking moieties derived from a crosslinking precursor selected from the group consisting of: a di(4-cyclooctenol) succinate, dicumyl peroxide (DCP), dibenzoyl peroxide (DBzP), di(tert-butyl) peroxide, and any combination thereof.

Aspect 100: The composite material of Aspect 74, wherein shape memory polymer comprises poly(cyclooctene), polycaprolactone, poly(lactic acid), poly(lactic-co-glycolic acid), polyethylene, polypropylene, thermoplastic polyurethane (TPU), or any combination thereof.

Aspect 101: A device comprising:
a composite material;
wherein the composite material comprises:
   one or more shape memory polymers; and
   a first additive provided in the one or more shape memory polymers; wherein:
   (a) the first additive comprises or is (optionally, is) a plurality of inorganic particles, (b) the first additive increases an ultrasound attenuation coefficient of the composite material (or at least of one or more portions thereof having the first additive) compared to that of the same one or more shape memory polymers free of first additive, and/or (c) the first additive comprises or is (optionally, is) a plurality of hollow particles;
   the composite material is characterized by the composite transition temperature (T_{cm,trans}); and
   the one or more portions of the composite material undergo the shape change from a temporary shape to a permanent shape when the one or more portions are heated to within 35° C. of T_{cm,trans} or a temperature approximately equal to or greater than T_{cm,trans}.

Aspect 102: A method of using a composite material, the method comprising:
directing one or more focused ultrasound beams at one or more portions of the composite material;
thereby, heating the one or more portions to a temperature approximately equal to or greater than a composite transition temperature (T_{cm,trans}); and
thereby, causing the composite material to undergo a shape change at the one or more portions thereof;
wherein the composite material comprises:
   one or more shape memory polymers; and
   a first additive provided in the one or more shape memory polymers; wherein:
   (a) the first additive comprises or is (optionally, is) a plurality of inorganic particles, (b) the first additive increases an ultrasound attenuation coefficient of the composite material (or at least of one or more portions thereof having the first additive) compared to that of the same one or more shape memory polymers free of first additive, and/or (c) the first additive comprises or is (optionally, is) a plurality of hollow particles;
   the composite material is characterized by the composite transition temperature (T_{cm,trans}); and
   the one or more portions of the composite material undergo the shape change from a temporary shape to a permanent shape when the one or more portions are heated to within 35° C. of T_{cm,trans} or a temperature approximately equal to or greater than T_{cm,trans}.

Aspect 103: A method of making a composite material, the method comprising:
polymerizing a monomer to form a first polymer;
crosslinking the first polymer in the presence of a crosslinking precursor and a first additive at a temperature approximately equal to or greater than a crosslinking temperature (T_{cm,crosslink}) to form the composite material having the crosslinked shape memory polymer and the first additive;
wherein the composite material comprises:
   the shape memory polymer; and
   the first additive provided in the shape memory polymer; wherein:
   (a) the first additive comprises or is (optionally, is) a plurality of inorganic particles, (b) the first additive increases an ultrasound attenuation coefficient of the composite material (or at least of one or more portions thereof having the first additive) compared to that of the same one or more shape memory polymers free of first additive, and/or (c) the first additive comprises or is (optionally, is) a plurality of hollow particles;
   the composite material is characterized by the composite transition temperature (T_{cm,trans}); and
   the one or more portions of the composite material undergo the shape change from a temporary shape to a permanent shape when the one or more portions are heated to within 35° C. of T_{cm,trans} or a temperature approximately equal to or greater than T_{cm,trans}.

Some of the following Examples pertain to synthesis of polymers, composites, or ingredients therefor.

### Example 1: Single Step Polymerization and Crosslinking of Cis-Cyclooctene to Make Composites

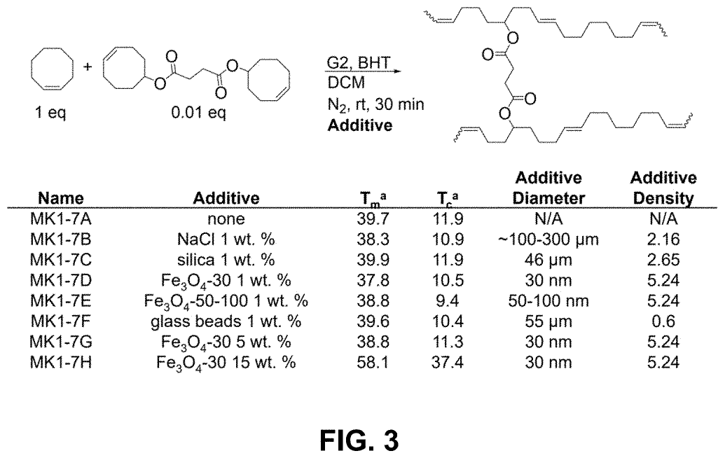

To a 20 mL scintillation vial containing the additive (1-15 wt. % relative to cis-cyclooctene+crosslinker) and a stir bar in a N₂ filled glovebox is added 1.026 mL of a mixture containing cis-cyclooctene (1.000 mL, 7.673 mmol, 1.0 eq.) and crosslinker (0.026 mL, 0.0767 mmol, 0.01 eq.). While stirring, 6.000 mL of a stock solution containing 2,6-Di-tert-butyl-4-methylphenol (0.2 mg, 0.0008 mmol, 0.0001 eq.) and G2, Benzylidene[1,3-bis(2,4,6-trimethylphenyl)-2-imidazolidinylidene]dichloro(tricyclohexylphosphine)ruthenium, (0.65 mg, 0.00077 mmol, 0.0001 eq.) dichloromethane (DCM) are combined. The solution is allowed to stir for 30 minutes, during which time gelation is observed. The vial is then removed from the glovebox and 6 mL of DCM containing 1 drop of ethyl vinyl ether is added under air. After 30 minutes, the solution above the gel is removed, and the gel is dried under vacuum on a Schlenk line for 48 hours to obtain a solid polymer sample, the color of which varies depending on the additive (FIG. 3).

Reagents used in this Example are commercially acquired and used as received except for as described here. cis-cyclooctene is stirred over CaH₂ for 24 hours and then distilled under vacuum, followed by storage in a Schlenk flask under Ar atmosphere until use in the above procedure. The crosslinker is synthesized from 4-cyclooctenol and succinic acid as follows.

Succinic acid (1 eq., 0.468 g, 3.962 mmol) and 4-dimethylaminopyridine (0.1 eq., 0.050 g, 0.3962 mmol) are dissolved in DCM (15 mL) under air. Triethylamine (3 eq., 1.700 mL, 11.886 mmol) is added to the solution at room temperature. At 0° C., 1-Ethyl-3-(3′-dimethylaminopropyl)carbodiimide hydrochloride (EDC-HCl, 2.8 eq., 2.127 g, 11.094 mmol) is added, followed by addition of cis-cyclooct-4-en-1-ol (2 eq., 1.0 g, 7.924 mmol). The reaction mixture is stirred at room temperature overnight. The mixture was diluted with DCM (30 mL) and an aqueous extraction is performed with water and brine. The product is purified with column chromatography on silica gel. 60% yield is obtained as a colorless oil. Purity is supported with ¹H nuclear magnetic resonance (NMR) spectroscopy. ¹H NMR (500 MHz, CDCl₃) δ 5.74-5.56 (m, 4H), 4.83 (td, J=9.5, 4.3 Hz, 2H), 2.55 (s, 4H), 2.39-2.26 (m, 2H), 2.19-2.06 (m, 6H), 1.94-1.79 (m, 4H), 1.75-1.67 (m, 2H), 1.64-1.54 (m, 6H).

### Example 2A: Cross-Linking of Poly(Cis-Cyclooctene) with Dicumyl Peroxide (DCP)

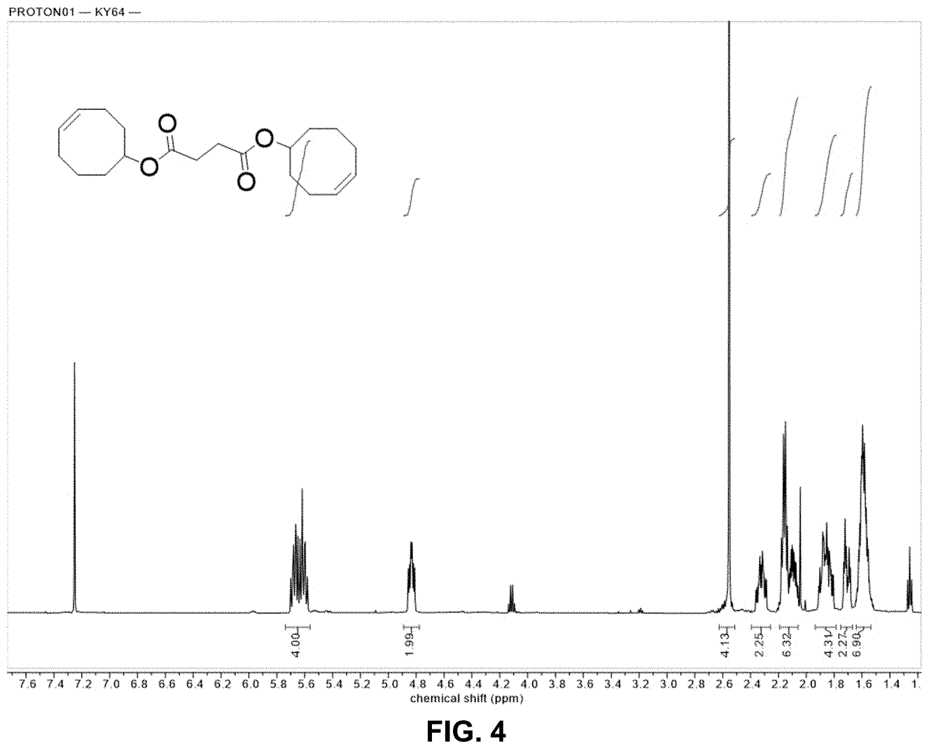

To a scintillation vial containing dicumyl peroxide (0.5-5 wt. % relative to poly(cis-cyclooctene, 0.005-0.050 g) is added poly(cis-cyclooctene) (Vestenamer, 1.000 g) and 10 mL toluene under air at room temperature. The mixture is stirred for 48 hours at which point it is noted to be a homogeneous viscous solution. The solvent is removed by rotary evaporation followed by drying for 48 hours on a Schlenk line. Dried polymer samples are placed in an oven at 130° C. for approximately 30 hours. Crosslinked samples are characterized by differential scanning calorimetry (DSC). (FIG. 4).

### Example 2B: Crosslinking poly(cis-cyclooctene)

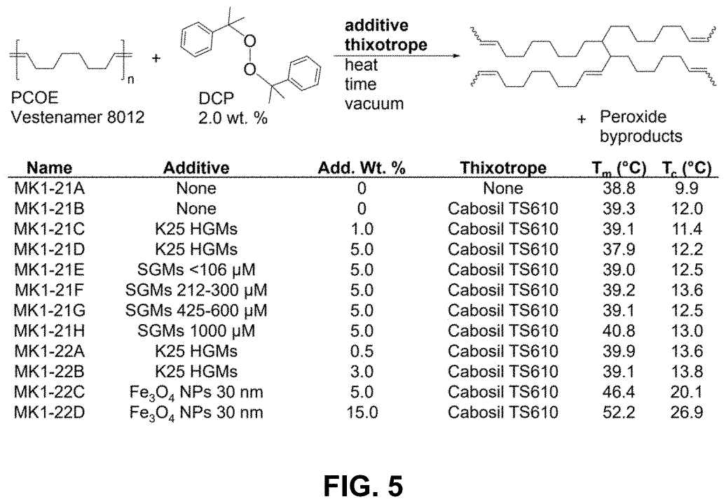

To a scintillation vial containing dicumyl peroxide (2.0 wt. % relative to poly(cis-cyclooctene, 1.000 g) is added poly(cis-cyclooctene) (Vestenamer, 1.000 g), an ultrasound absorbing additive (0.5-15 wt. %), a thixotrope (Cabosil TS610, 1.0 wt. %), and 10 mL toluene under air at room temperature. The mixture is stirred for 48 hours at which point it is noted to be a heterogeneous viscous solution where only the additive remains undissolved. The vial is agitated until the suspension is homogeneously mixed and the solvent is immediately removed by rotary evaporation followed by drying for 48 hours on a Schlenk line. Dried polymer samples are placed in a suitable mold and crosslinked in a vacuum oven (set to 150° C. for approximately 43 hours. Crosslinked samples are characterized by differential scanning calorimetry (DSC) (FIG. 5).

Some of the follow Examples pertain to shape memory performance and thermal characterization.

### Example 3: DSC Example Thermogram and Procedure

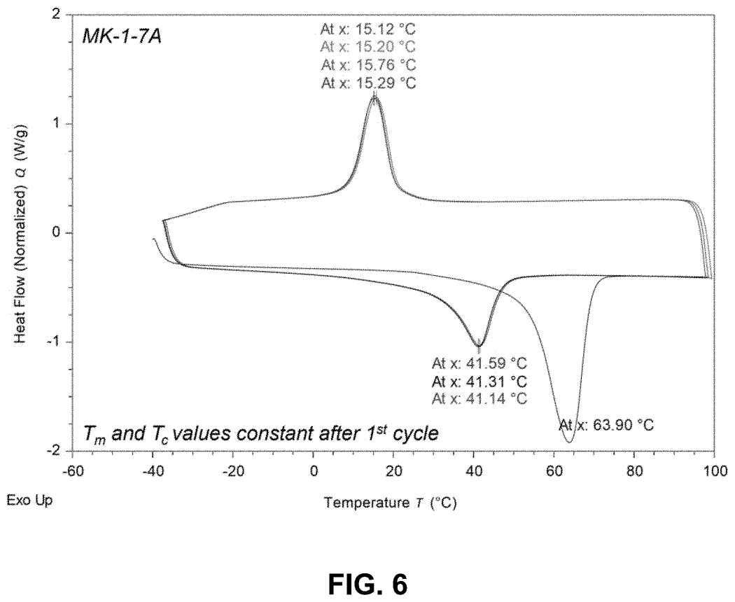

A dried polymer sample synthesized as described in Example 1 and weighing 7.6 mg is placed in an aluminum pan and hermetically sealed. DSC is performed at a ramp rate of 10° C./min for the indicated number of cycles between −40.0° C. and 100.0° C. (FIG. 6) using a TA instruments DSC-25. Air is used as the purge gas.

### Example 4: Relationship Between DSC Results and Sample Processing

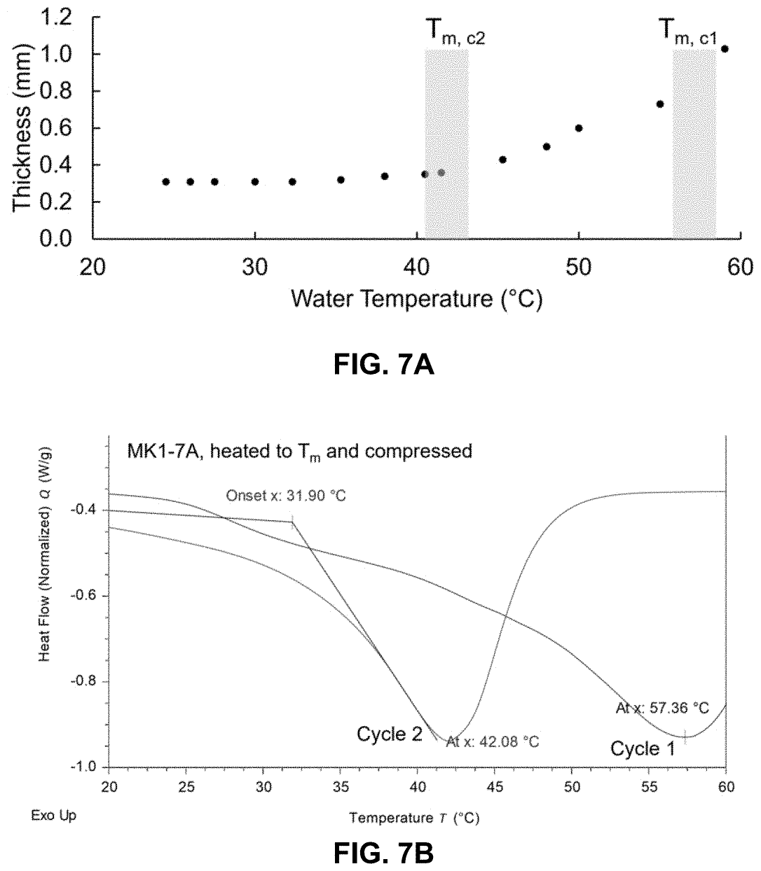

A sample prepared as in Example 1 with no additive (MK-1-7A) is heated to Tₘ in a 60° C. metal bead bath and then compressed while hot in custom clamp (see below for details). The sample is allowed to cool to room temperature and is cut to a rectangular sheet for DSC (weight 18.7 mg). DSC is run to test the hypothesis that the Tₘ obtained from repeated DSC cycling is in fact the temperature at which shape memory occurs. The sample processed for DSC reflects the processing commonly used for shape memory testing. In a second experiment, a sample from the same batch of polymer is heated to Tₘ and compressed from thickness 1.83 mm to 0.33 mm using the custom clamp without the mold insert piece. The flattened sample is equilibrated for 5 minutes in a water bath at increasing temperatures and the thickness is measured afterwards using calipers. The resulting plot of thickness vs. temperature is compared with the DSC results (FIGS. 7A-7B). In embodiments, the shape memory effect does not begin until after the true Tₘ (cycle 2) is exceeded. A more significant change in thickness is observed as the temperature approaches the cycle 1 Tₘ, that obtained without erasure of the thermal history.

### Example 5A: Tₘ is Controllable Through Varying Crosslinker Loading

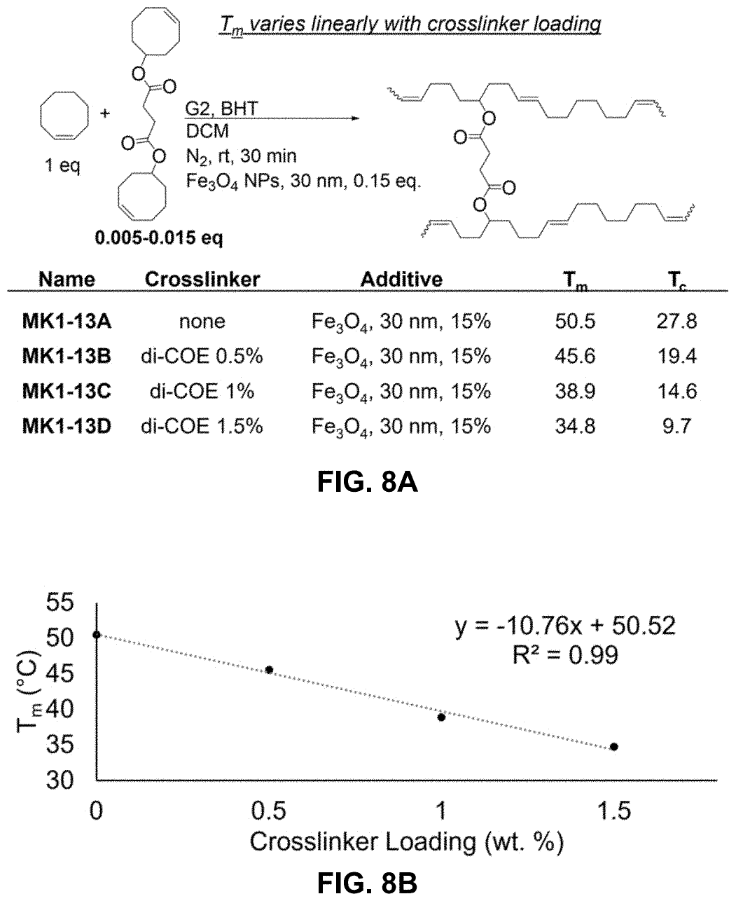

Composite samples are synthesized as in Example 1 with varying crosslinker loading from 0.5-1.5 mol % relative to monomer. Resulting samples are characterized by DSC and found to exhibit a linear relationship between Tₘ and crosslinker loading (FIGS. 8A-8B).

### Example 5B: Heat Triggered Shape Memory Response of a Compressed Sample

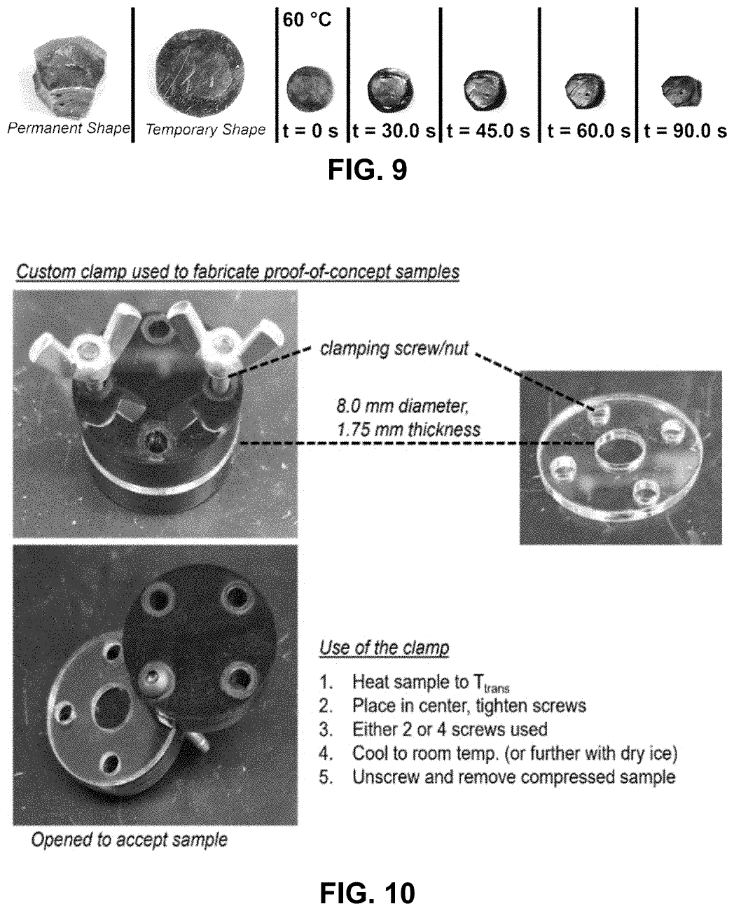

Shape memory is observed in a composite material, crosslinked poly(cis-cyclooctene) containing Fe₃O₄ nanoparticles, prepared as shown in FIG. 7A, MK1-13D. The nanoparticles are included in the polymer to enhance ultrasound induced heating. The material is cut into a roughly cylindrical shape of diameter 5.74 mm and height 4.98 mm. The material is then heated to Tₜᵣₐₙₛ by placing it in an oven at 130° C. for 5 minutes. While hot, the material is compressed using a poly(methyl methacrylate) screw clamp (custom made, see below) with an 8 mm diameter mold insert. Cooling to room temperature and then over dry ice for 10 minutes results in a flat, cylindrical sample of 8.14 mm diameter and 1.93 mm height. Allowing the sample to equilibrate at room temperature completes fabrication and shape memory programming of sample. The sample is placed in a glass dish in a metal bead bath set to 60° C. and observed to contract in diameter, restoring the original dimensions (final dimensions 5.90 mm diameter×4.95 mm height, FIG. 9).

A custom screw clamp is fabricated from poly(methyl methacrylate) by laser cutting and includes 3 pieces (FIG. 10). The 4 outer holes can accommodate up to 4 M3 screws and wing nuts. The optional interior piece with an 8 mm diameter hole may be used to mold the material to create a 2 mm thickness disc.

Some of the following Examples pertain to ultrasound induced heating and the shape memory response.

### Example 6: Ultrasound Triggered Shape Memory Unfolding

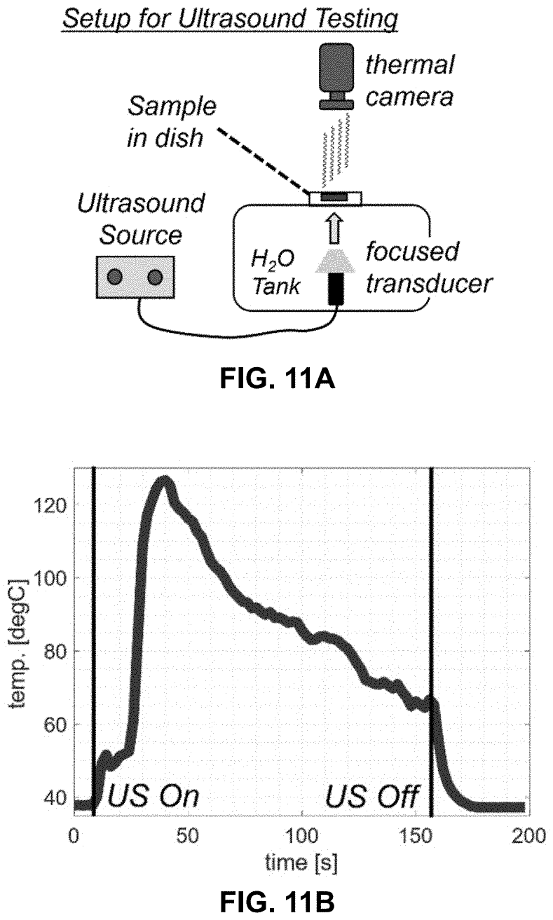

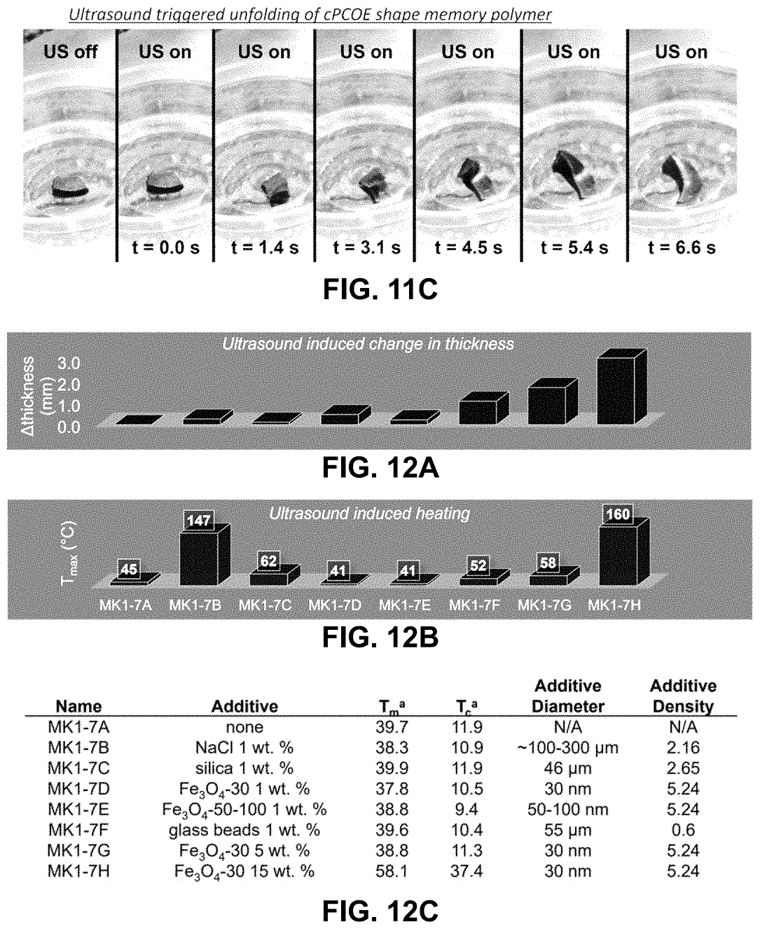

A sample prepared as in FIG. 3 (MK1-7H) is programmed to its temporary shape as follows. The material is cut into a rectangular strip of dimensions 3.45 mm×9.87 mm×1.37 mm. The material is then heated to Tₜᵣₐₙₛ by placing it in an oven at 130° C. for 5 minutes. While hot, the material is folded 180° and compressed using a poly(methyl methacrylate) screw clamp (custom made, see above). Cooling to room temperature completed fabrication and shape memory programming. The sample is tested in an apparatus (See FIG. 11A) having a focused ultrasound transducer (Precision Acoustics Model 960) operated at 30 V input voltage and 670 kHz aimed upwards towards a sample stage in a temperature-controlled water tank, and a thermal camera (FLIR A655sc) is placed aiming downwards perpendicular to the sample. The ultrasound power is, for example, approximately 2.7 W cm⁻². The sample stage holds a Petri dish with a Mylar film bottom that is transparent to ultrasound. The water level comes up to the bottom of the dish and the temperature is set to 37.0° C. H₂O is pipetted into the well in the bottom of the dish (˜1 mL) and the sample is placed in the thin layer of fluid. Upon ultrasound stimulation, the sample is observed to unfold within a period of ˜7 seconds (FIG. 11C). The temperature of the sample as recorded with the thermal camera rises to ˜50° C. quickly and is stable at that temperature during the period of shape change. After shape change is complete, the sample temperature continues to rise. Notably, the surrounding water does not change significantly in temperature throughout the process.

### Example 7: Ultrasound Triggered Heating and Shape Memory Thickening

Samples from each of the composites synthesized as in FIG. 3 are each cut into a roughly circular shape of ˜4 mm thickness and heated to Tₜᵣₐₙₛ in an oven at 130° C. for 5 minutes. While hot, the material is compressed using a poly(methyl methacrylate) screw clamp (custom made, see above) with the 8 mm disc molding insert. Samples are cooled to below room temperature on dry ice and then allowed to equilibrate at room temperature. The 8 mm discs are then exposed to ultrasound using the setup described in Example 6. Temperature of the samples is monitored and the thickness of each sample is measured before and after ultrasound exposure (FIGS. 12A-12C). From the results, it can be observed that increased loading of the Fe₃O₄ nanoparticles is associated with an increase in maximum temperature reached under ultrasound exposure. In some cases, at the lowest loading of 1 wt. %, only additives with particle diameter in the 10-1000 μm range show a significant heating response. In some cases, high density alone is insufficient to elicit a strong heating response as indicated by the Fe₃O₄ nanoparticle samples at 1 wt. %.

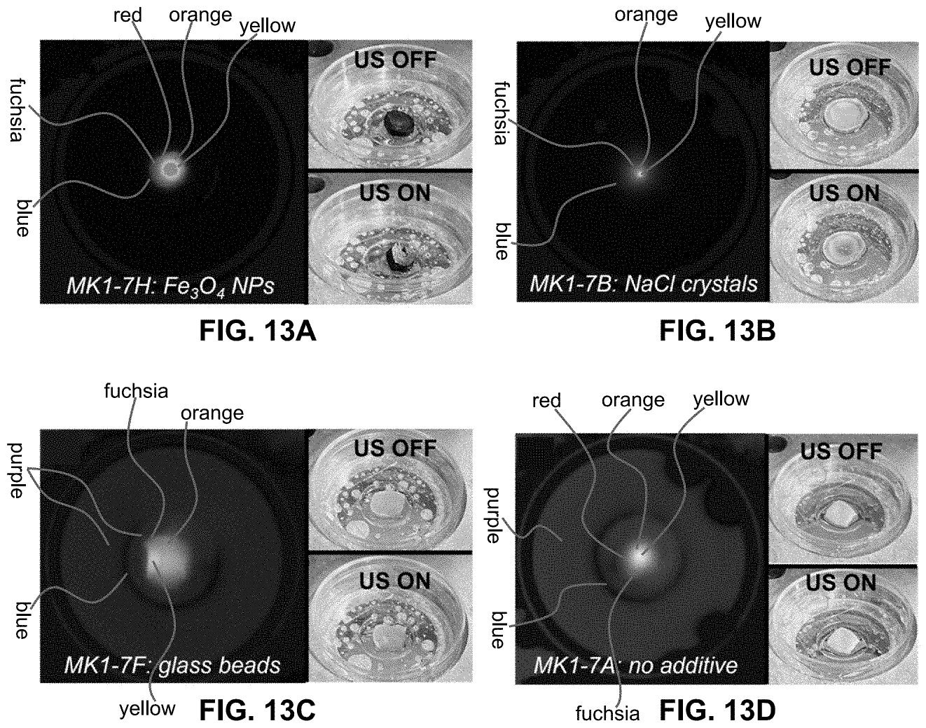

The additive chosen also affected the localization of the heating response (FIGS. 13A-13D). Compared to the no additive control (FIG. 13D), the presence of glass beads (FIG. 13C) in the sample leads to a wider area over which the maximum temperature is distributed, while the presence of NaCl crystals (FIG. 13B) leads to a very narrow region of maximum temperature. The NaCl sample also visually shows a localized heating effect in the photographs manifested by a small translucent region that results from the melt state being reached. Individual crystals in the NaCl sample are observable in the center of the melted region, indicating that the heating effect may depend on additive location. In contrast, the sample with glass beads appears uniform in the photograph. In some cases, the amount of shape memory thickness increase observed for the samples can be limited in the case of NaCl despite the high maximum temperature reached. Likely, this may be due to the low temperature of surrounding polymer chains maintaining crystallization and thus preventing bulk shape change. As such, the use of NaCl as an additive can result in limiting shape change to localized regions, which is advantageous for some applications contemplated herein.

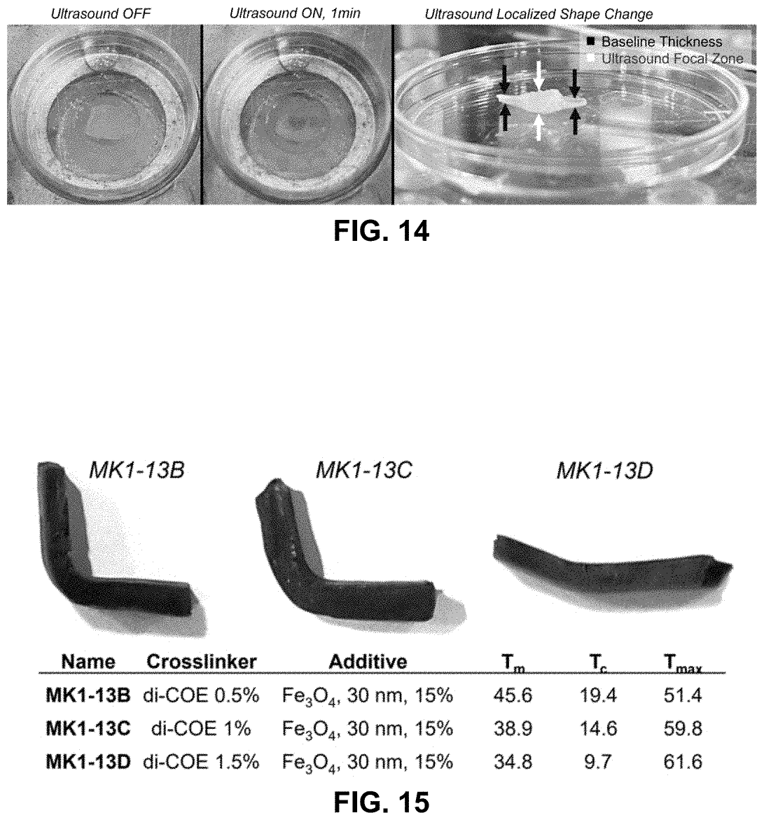

More broadly, in various aspects, the shape change is localized to the region in which heating meets or exceeds Tₜᵣₐₙₛ. In support of this observation, a sample containing silica gel as additive is prepared as above, except it was compressed further to form a flattened disc. Photographs of the sample during and after ultrasound exposure confirm that the region of shape change is limited to the region of heating, which in this case occurs only within the area of ultrasound focus (FIG. 14).

### Example 8: Effect of Crosslinker Loading and Tₘ on Extent of Ultrasound Induced Shape Change

Composites prepared as described in FIGS. 8A-8B containing varying crosslinker loading are cut into straight rectangular rods with a razor. Samples are heated in an oven at 130° C. for 5 minutes and then bent over a metal 90° template while hot. The template is cooled on dry ice. The samples are then allowed to equilibrate at room temperature, completing shape programming. Samples are placed in the same ultrasound testing setup as described above, oriented vertically, with one end perpendicular to the Petri dish, and ultrasound is applied for 1 minute. Photographs are taken after ultrasound exposure (FIG. 15). The sample with the highest crosslinker loading and lowest Tₘ show the greatest degree of shape recovery in this experiment. The heating response is higher with the 1% and 1.5% loading samples. Notably, when ultrasound is turned off after 1 minute in this example, the shape memory process did not continue. As such, intermediate shape recovery is demonstrated and may be controllable by tuning the ultrasound exposure time.

### Example 9: Cross-Linking of Poly(Cis-Cyclooctene) with Dibenzoyl Peroxide (DBzP)

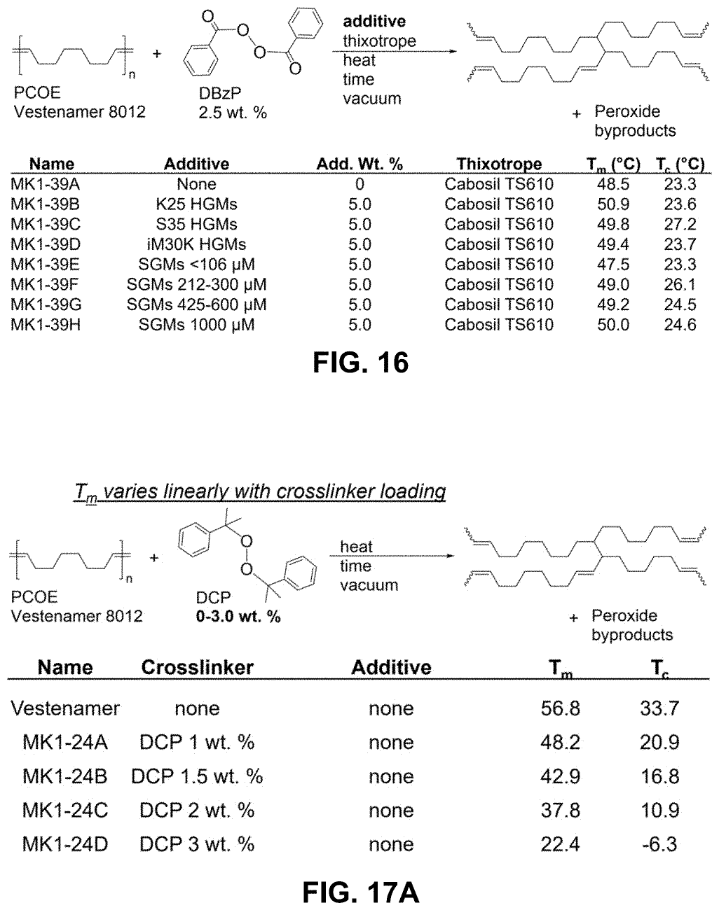

To a scintillation vial containing poly(cis-cyclooctene) (Vestenamer, 1.000 g), an ultrasound absorbing additive (5.0 wt. %, relative to polymer), a thixotrope (Cabosil TS610, 1.0 wt. %), dibenzoyl peroxide (2.5 wt. %), and 10 mL toluene are added under air at room temperature. The mixture is stirred for 48 hours at which point it is noted to be a heterogeneous viscous solution, where only the additive remains undissolved optionally. The vial is agitated until the suspension is homogeneously mixed and the solvent is immediately removed by rotary evaporation followed by drying for 48 hours on a Schlenk line. Dried polymer samples are placed in a suitable mold and crosslinked in a vacuum oven (set to 140° C. for approximately 18 hours. Crosslinked samples are characterized by differential scanning calorimetry (DSC) (FIG. 16).

### Example 10: Tₘ is Controllable with Dicumyl Peroxide Loading

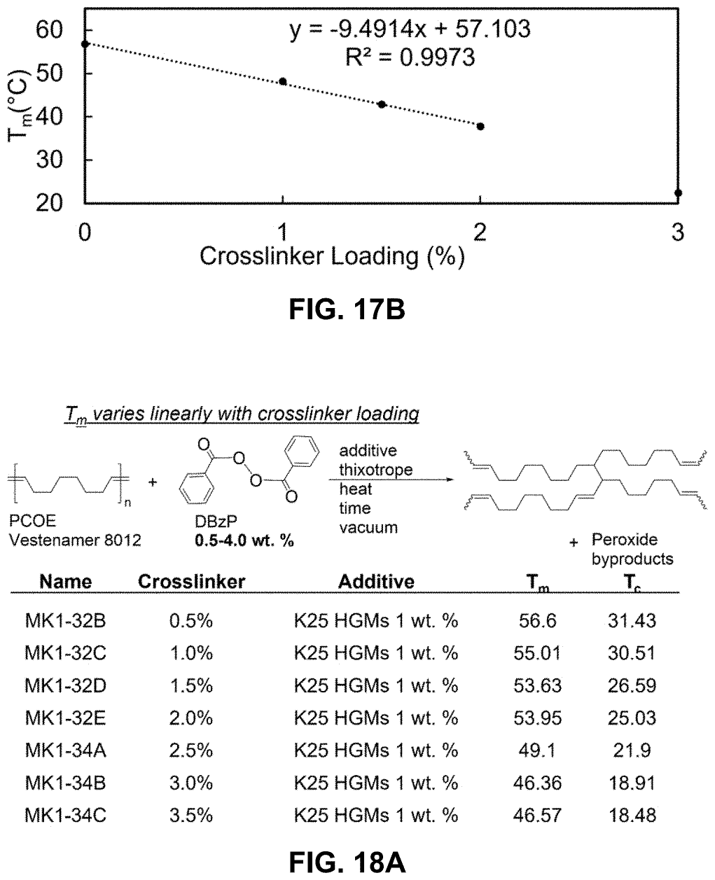

Polymer samples are synthesized as in Example 2B without additive or thixotrope and with varying crosslinker loading from 0-3.0 wt. % of DCP relative to polymer. Resulting samples are characterized by DSC and found to exhibit a linear relationship between Tₘ and crosslinker loading (FIGS. 17A-17B).

### Example 11: Tₘ is Controllable with Dibenzoyl Peroxide Loading

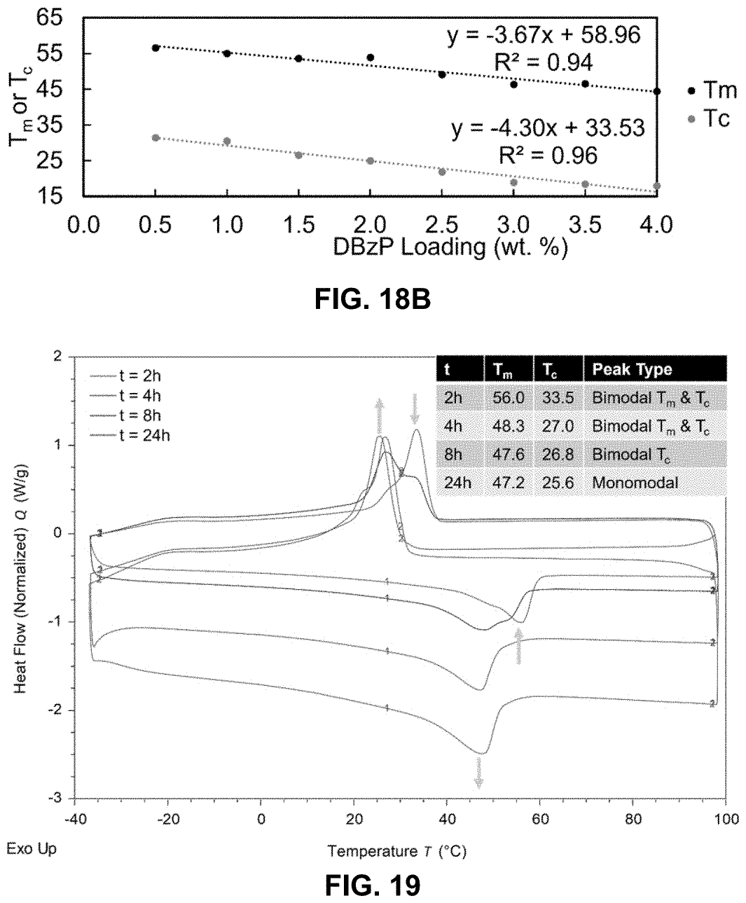

Polymer samples are synthesized as in Example 9 with varying crosslinker loading from 0.5-4.0 wt. % of DBzP relative to polymer. Resulting samples are characterized by DSC and found to exhibit a linear relationship between Tₘ or T_{c} and crosslinker loading (FIGS. 18A-18B).

The kinetics of the crosslinking reaction are followed by making samples as in Example 9 containing 2.5% DBzP but varying the time of crosslinking in the vacuum oven (2 h, 4 h, 8 h, and 24 h time points). The resulting samples are characterized by DSC and found to exhibit bimodal melting and crystallization curves when crosslinking is incomplete. Only the 24 h time point show monomodal peaks for both Tₘ and T_{c}, suggesting that the time for complete curing is 8 h<t_{cure}<24 h. (FIG. 19)

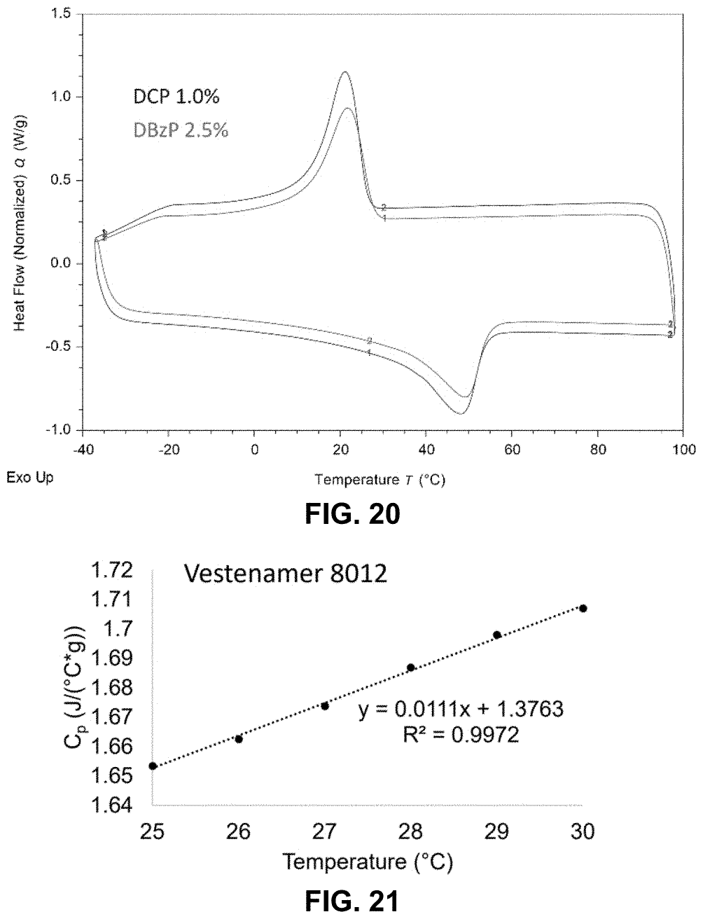

Lastly, a polymer is prepared with approximately the same thermal characteristics (Tₘ and T_{c}) using either DCP or DBzP as crosslinker by varying crosslinker loading. This agreement suggests that these crosslinkers may be used interchangeably to make composites with targeted and controlled melting and crystallization transitions (FIG. 20).

### Example 12: Specific Heat Capacity of Vestenamer 8012

Calibration for modulated DSC (mDSC) is completed with a sapphire standard (TA Instruments, 21.97 mg). A quasi-isothermal mDSC run is performed with a Vestenamer sample (9.9 mg) at increasing temperatures with 1° C. steps from 25-30° C. Temperature is modulated by 1° C. for 60 s, followed by a 15 min isothermal period in which data was collected during the last 10 minutes. The average Cₚ value for each 10 min segment is plotted with a linear fit (FIG. 21).

### Example 13: Ultrasound Attenuation Coefficient of Vestenamer 8012

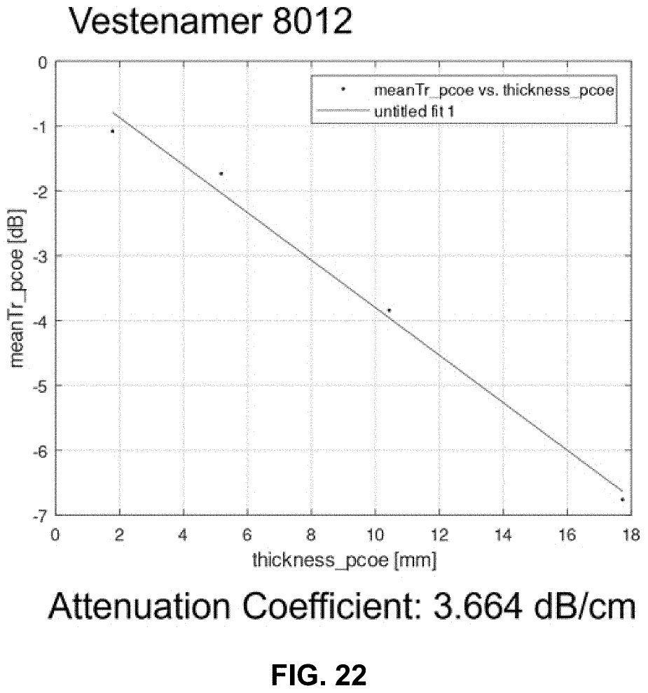

Four cylindrical samples of increasing thickness (1.78-17.73 mm) and constant diameter are molded from Vestenamer 8012. Samples are placed in a 3D-printed holder inline with a hydrophone and a 550 kHz ultrasound transducer. The hydrophone detects ultrasound transmitted by the sample. The average transmission is plotted as a function of thickness, providing an attenuation coefficient of 3.664 dB/cm. (FIG. 22)

### Example 14: Tensile Testing of Vestenamer 8012

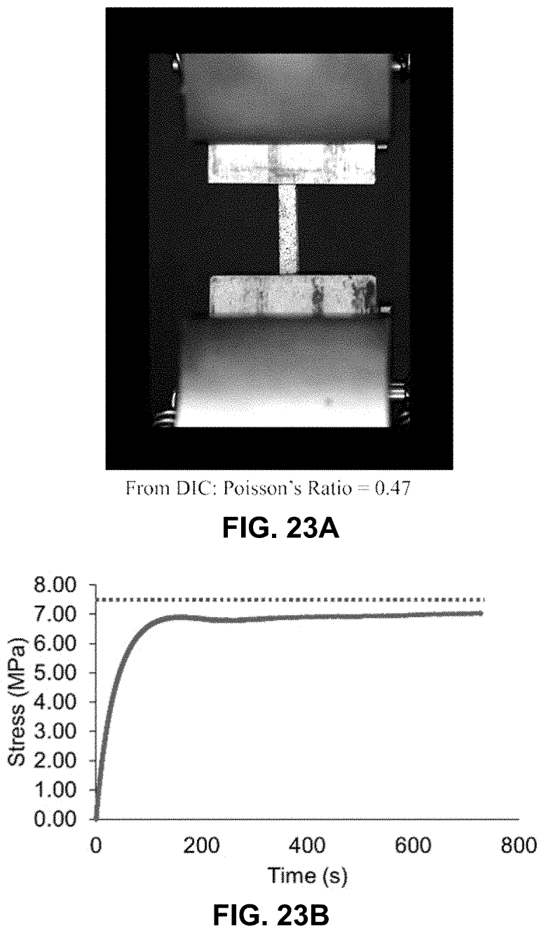

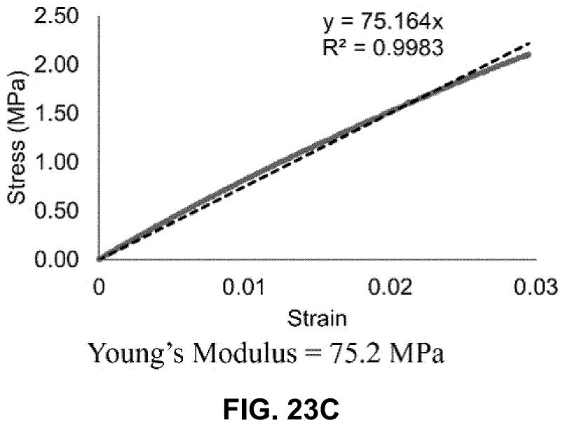

Vestenamer 8012 is molded to form an ASTM type C dogbone specimen which is analyzed using a uniaxial tensile testing setup (Instron) with a 250 N load cell. The sample was speckled with black spray paint and high-resolution photographs are acquired during the test (Pentax K-1 camera equipped with 100 mm macro lens). The photographs are used with digital image correlation software (Vic) to extract Poisson's ratio. (FIGS. 23A-23C)

### Example 15: Thermal Conductivity Measurement of Vestenamer 8012

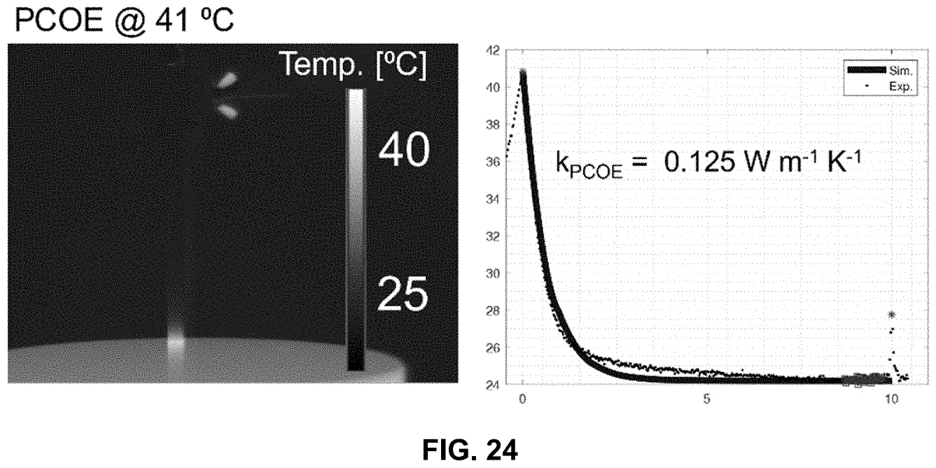

A sample of Vestenamer 8012 is molded into a rectangular prism (1.0×0.5×10.0 cm) and placed on the edge of a hot plate set to 41° C. The temperature is measured using a thermal camera (FLIR A655sc) and the resulting data is compared with simulated data (COMSOL Multiphysics). The thermal conductivity of the simulation that best fit the data is, for example, 0.125 W m⁻¹ K⁻¹. (FIG. 24)

### Example 16: Ultrasound Induced Heating and Shape Memory with DCP Composites

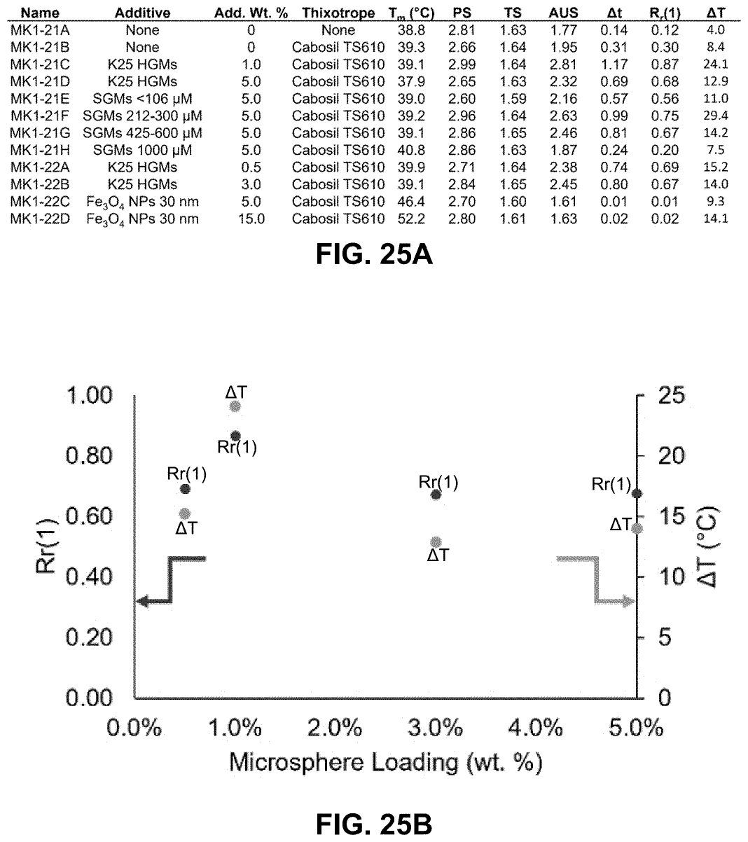

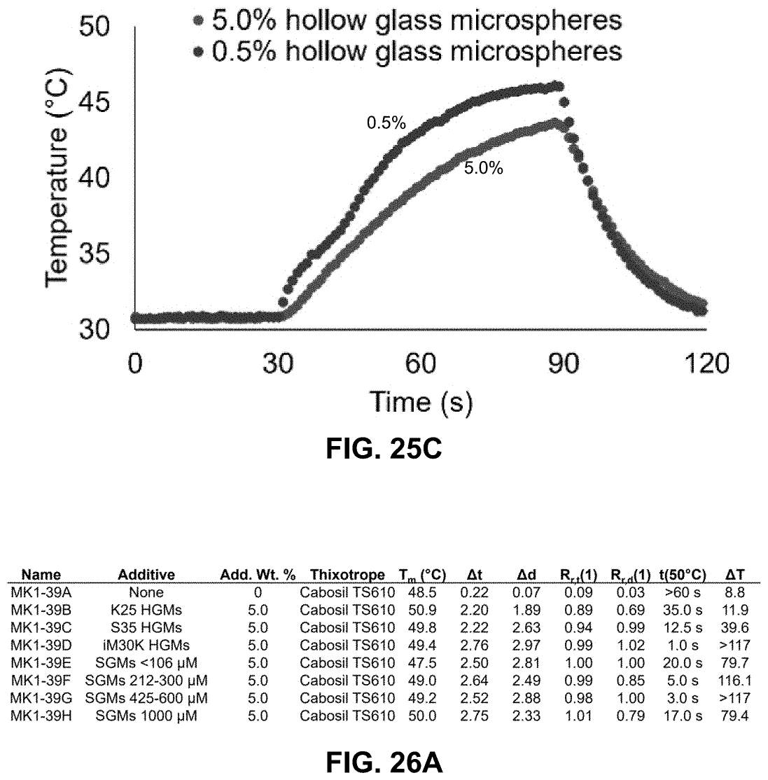

The 12 samples are prepared as in Example 2B using a drilled Teflon mold to make cylindrical samples of diameter 5.0-5.2 mm and thickness 2.7-3.0 mm. Samples are heated beyond Tₘ to ˜70° C. in an oven and compressed with the custom clamp (FIG. 9) using the 8 mm diameter insert to form discs of thickness 1.63+/−0.02 mm as the temporary shape. These discs are placed in a Petri dish in the ultrasound setup described in Example 6 and exposed to ultrasound for 1 minute each. The heating and shape memory responses are quantified and appear in FIG. 25A. For shape memory, Rᵣ(1) is the strain recovery ratio for 1 shape memory cycle defined as:

$${R}_{r} ( 1 ) = \frac{{ε}_{m} - {ε}_{p}}{{ε}_{m}} where {ε}_{m} = \frac{{t}_{1} - {t}_{2}}{{t}_{1}} and {ε}_{p} = \frac{{t}_{1} - {t}_{3}}{{t}_{1}}$$

Here t₁ is the thickness of the permanent shape, t₂ is the thickness of the temporary shape, and t₃ is the thickness after the ultrasound experiment. Samples are exposed to ultrasound in the setup described in Example 6 except with the bath temperature set to 32.0° C. to accommodate the lower Tₘ of these samples. The Aptflex standard is measured in between each sample and exhibited a heating profile that varied by up to 18.3%. This variation is in response to bubble buildup on the transducer. Loading of beads does not appear to significantly affect the temperature response in terms of maximum temperature reached or the resulting strain recovery ratio Rᵣ. However, there is a significant difference in heating kinetic profile, where the sample containing more K25 HGMs heated and cooled at a slower rate. (FIGS. 25A-25C)

### Example 17: Ultrasound Induced Heating and Shape Memory with DBzP Composites

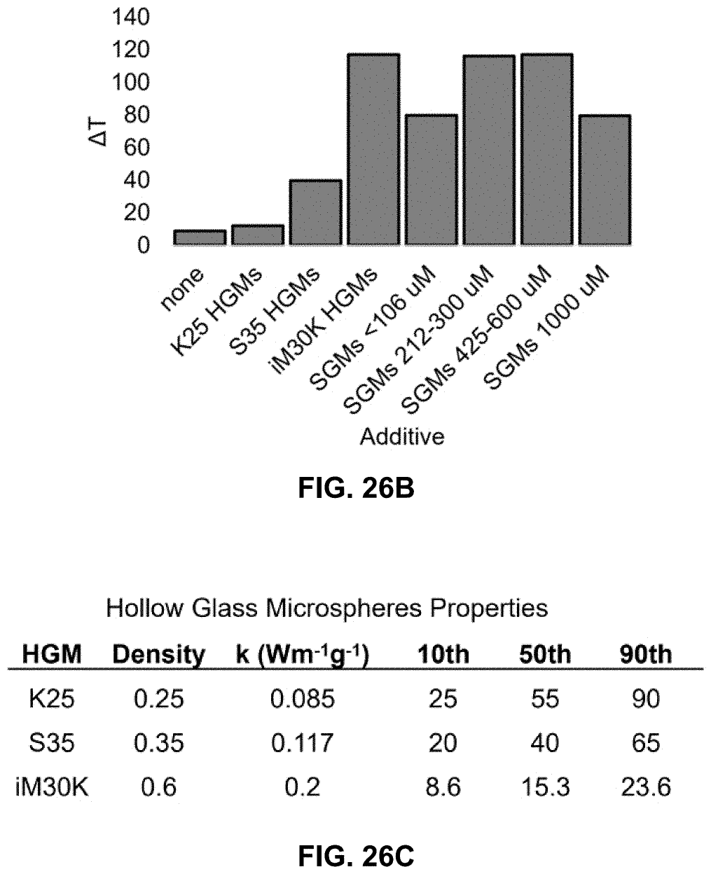

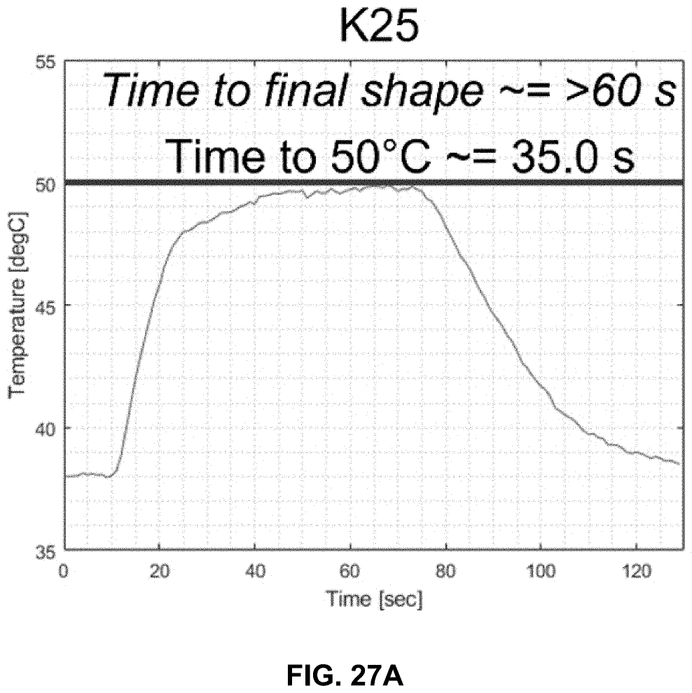

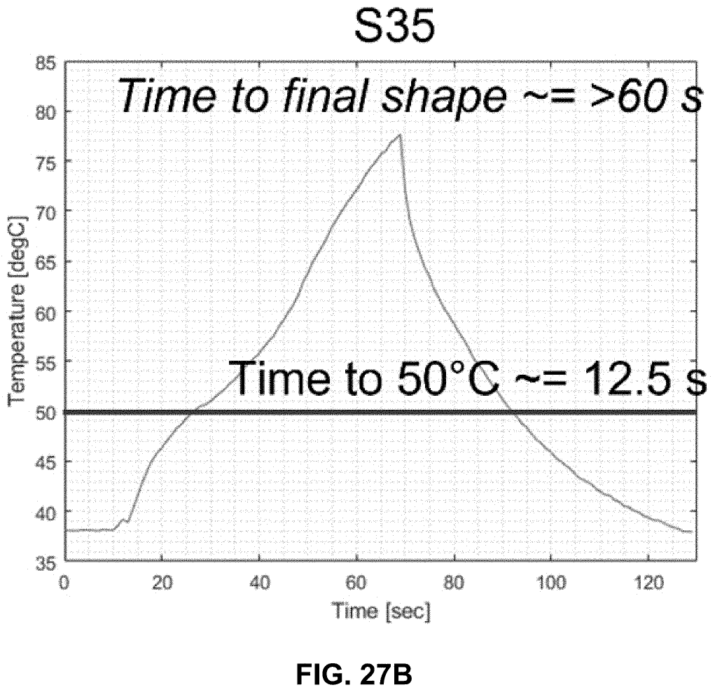

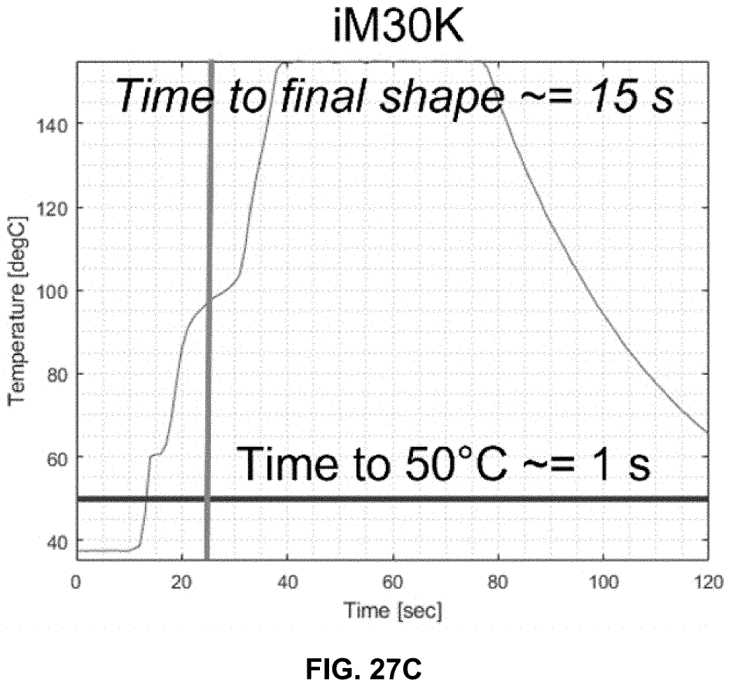

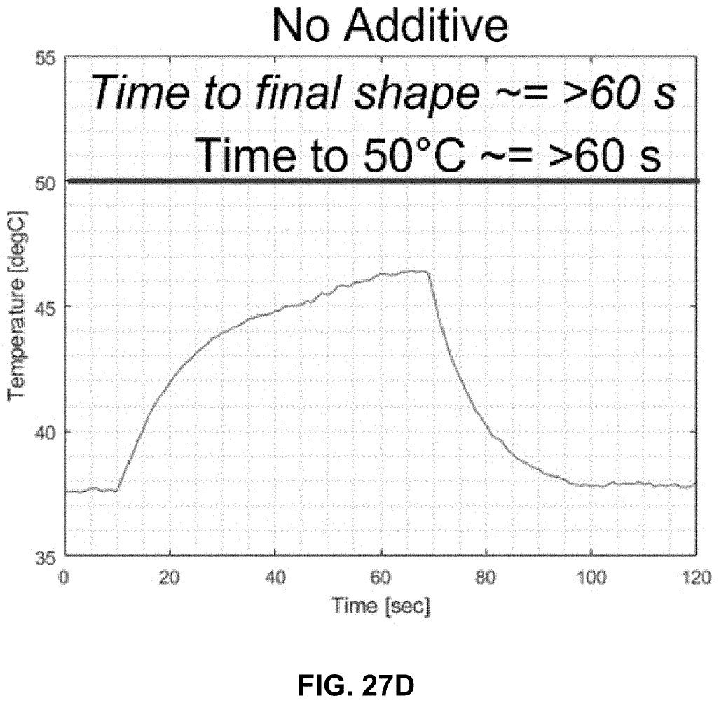

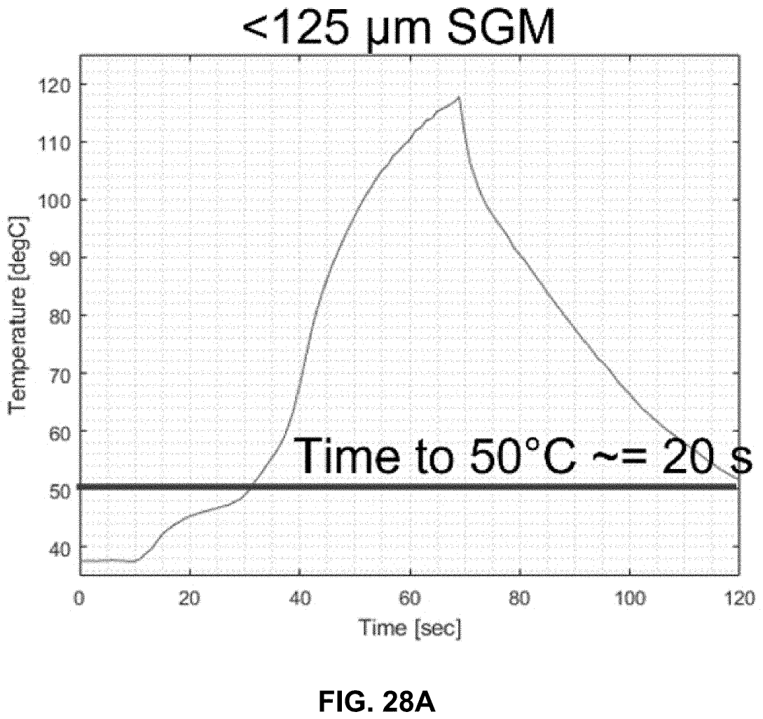

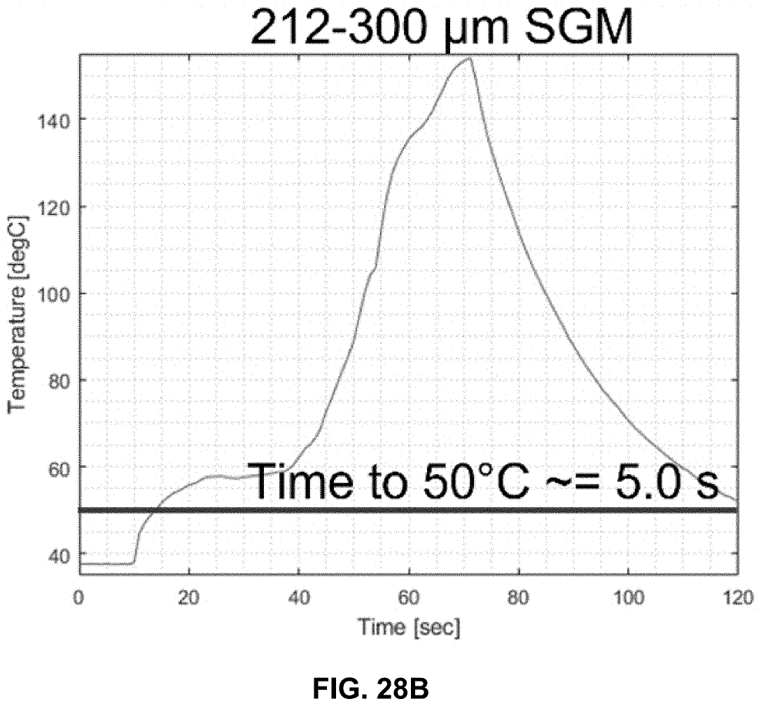

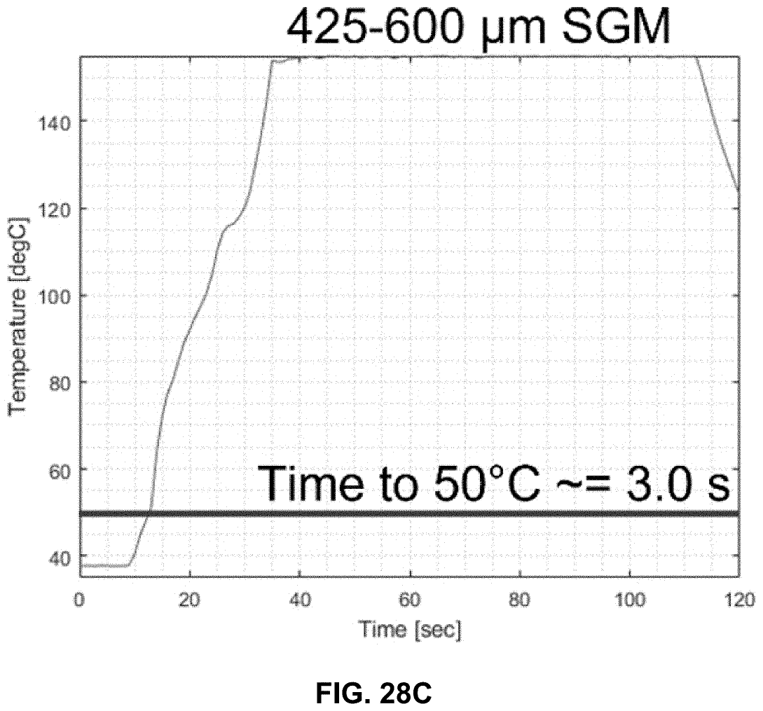

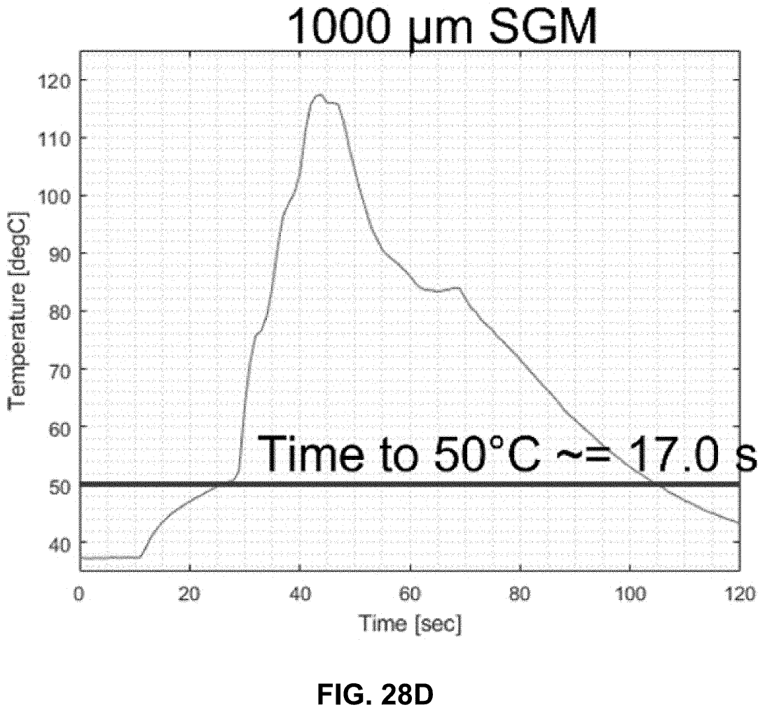

Eight (8) samples prepared as in Example 9 are crosslinked using a drilled teflon mold to make cylindrical samples of diameter 5.0-5.2 mm and thickness 4.0-4.5 mm. Samples are heated beyond Tₘ to −70° C. in an oven and compressed with the custom clamp (FIG. 9) using the 8 mm diameter insert to form discs of thickness 1.70+/−0.02 mm and diameter 7.94+/−0.11 mm as the temporary shape. These discs are placed in a Petri dish in the ultrasound setup described in Example 6 with the water bath set to 37° C. and exposed to ultrasound for 1 minute each. The Aptflex standard is measured in between each sample and exhibited a heating profile that varied by up to 3.8%. This variation is significantly less than in the previous experiment due to use of a water circulating pump to prevent bubble buildup on the transducer. It is noted that the sample containing no additive but with thixotrope compared well with previous results (Example H) with temperature rise 8-9° C. in both cases, despite different crosslinker (DBzP vs. DCP) and thermal properties (Tₘ, T_{c}). The higher Tₘ and T_{c} in the DBzP case is targeted to minimize shape change in the absence of ultrasound with Tₘ˜10-14° C. above body temperature. Compared to this control sample, samples containing HGMs or SGMs show significantly enhanced ultrasound responses, both in terms of heating (ΔT and heating rate) and shape memory (R_{r,t}(1) and R_{r,d}(1)). In particular, the composites containing iM30K HGMs showed the greatest heating response, exceeding the upper limit of the thermal camera at 155° C. and heating to 50° C. in the shortest time (˜1 s). iM30K HGMs also led to the fastest shape memory, achieving complete recovery of the permanent shape in only 15 s as measured by video camera (Samsung Note 20 Ultra 5G). In the SGM series, a maximum temperature response is observed in the 425-600 μm sample, with larger and smaller SGMs showing reduced response. Overall, iM30K HGMs can be concluded to be the most sensitive additive identified tested according to this Example 17, with the largest temperature response and fastest shape memory response. (FIGS. 26A-26C, FIGS. 27A-27D and FIGS. 28A-28D)

### Example 18: A Self Tightening Knot—Demonstrating Low Thermal Exposure to Surroundings with Successful Shape Change

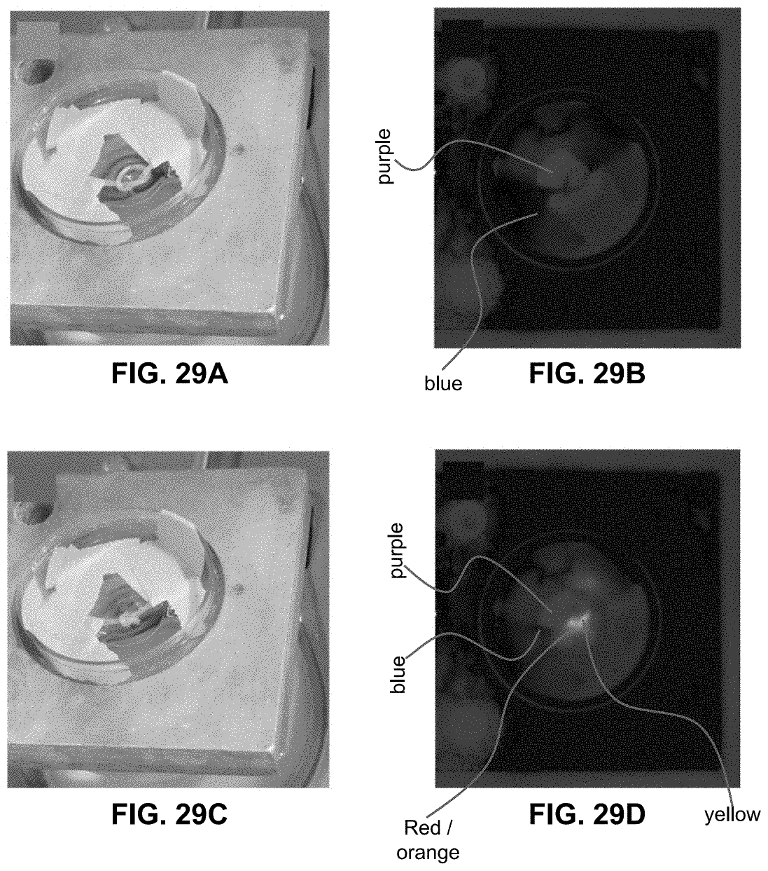

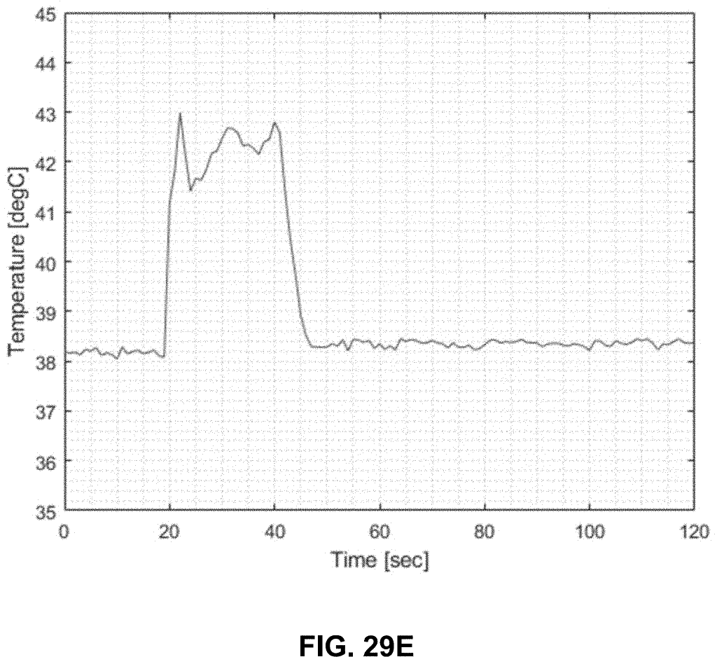

A narrow rod composite is fabricated as in Example 9 containing 1 wt. % K25 HGMs. The sample is heated to 70° C. in an oven, pulled to approximately 100% strain, tied in an overhand knot, and allowed to cool to room temperature to complete programming. The programmed sample is secured to a dish without a mylar film (open on the bottom) using masking tape. The sample is exposed to ultrasound in the typical setup, except it is in direct contact with the water bath and half submerged. The sample is observed to self-tighten while the temperature monitored via thermal camera never exceeded 43.0° C. Importantly, high temperatures are localized on the composite sample. The bath temperature changed by less than 0.2° C. during the test. The water bath surroundings are contemplated to behave similarly to tissue in terms of heat dissipation. As such, shape change is demonstrated in this experiment without exposure of surroundings to dangerous temperatures. (FIGS. 29A-29E)

### Example 19: Simulations of Ultrasound Induced Heating of Composites in Tissue

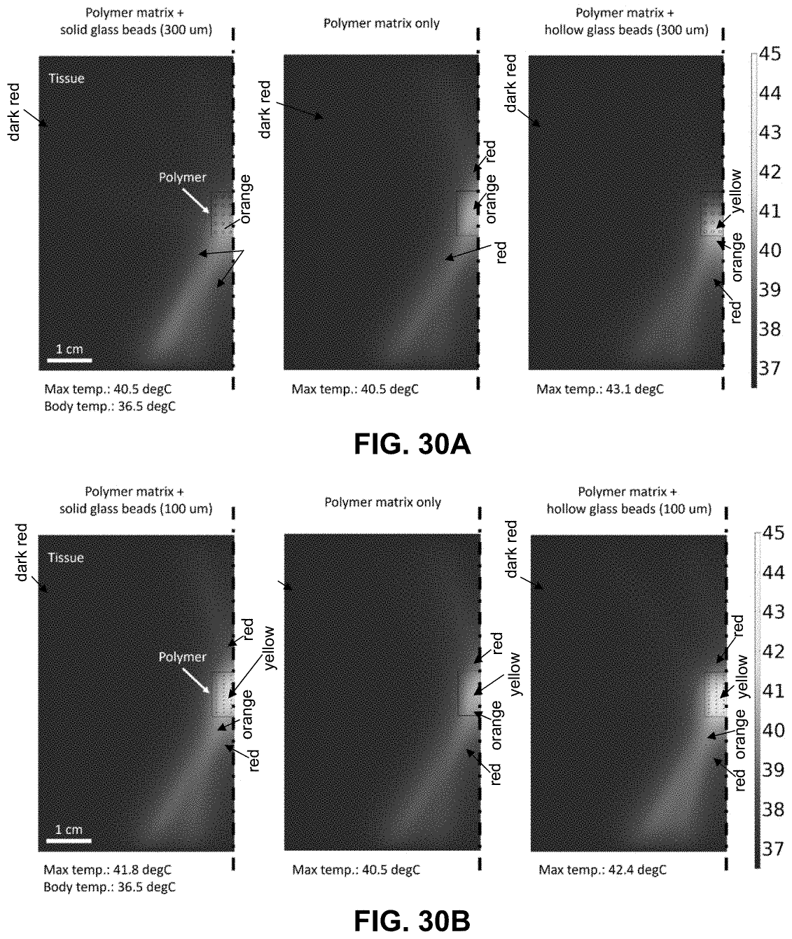

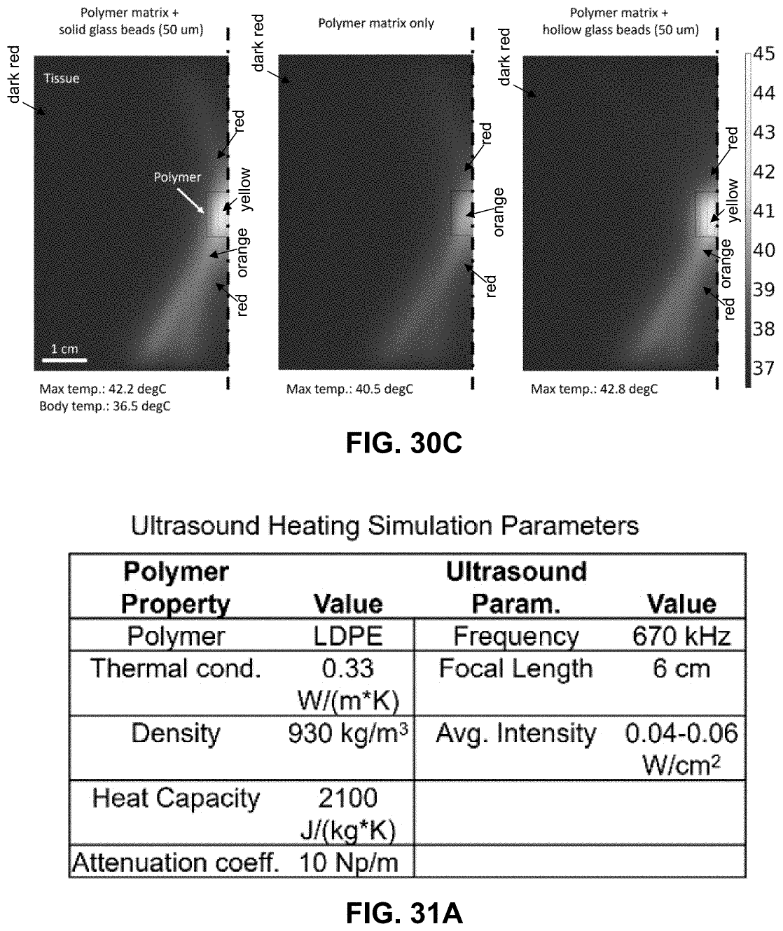

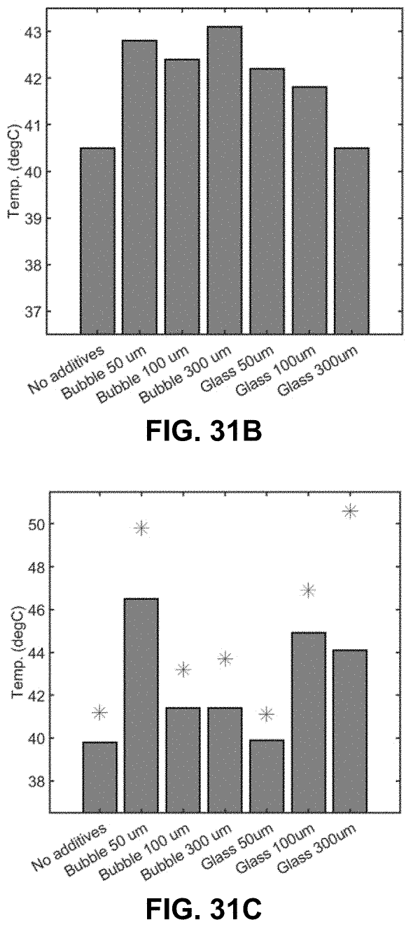

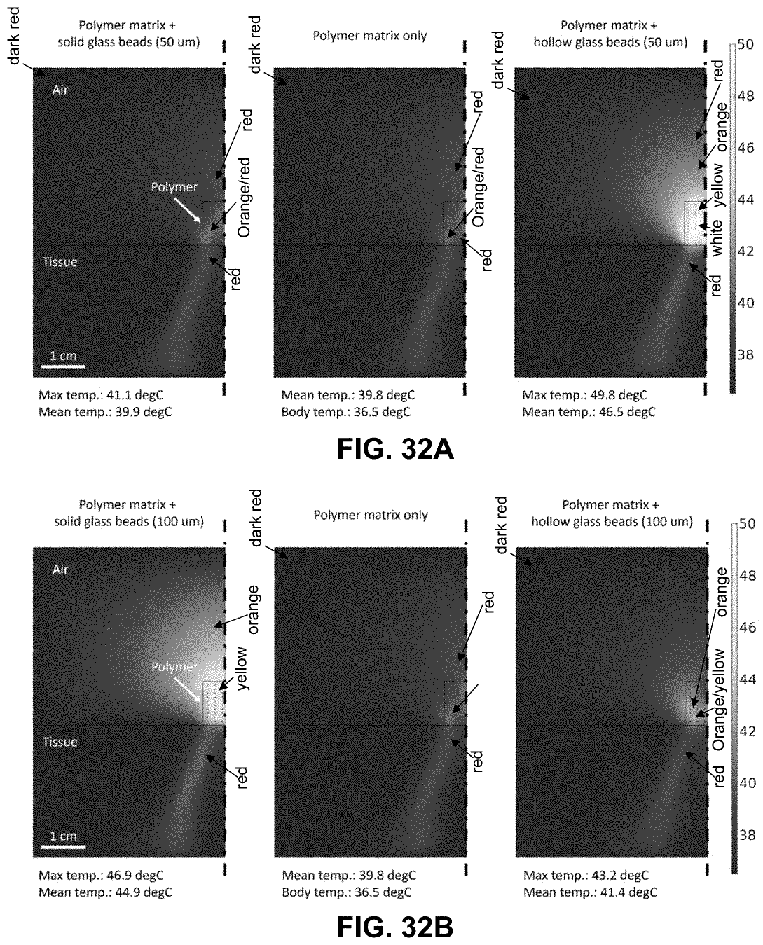

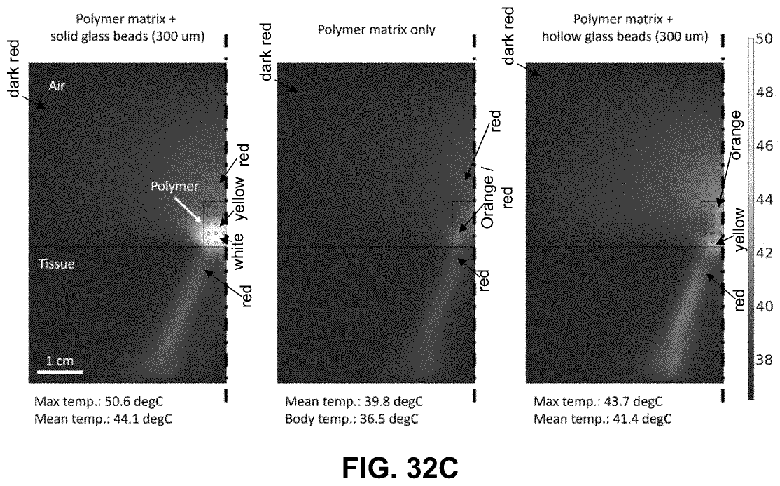

2D Simulations are performed using COMSOL Multiphysics with a symmetric boundary condition on the right edge. A polymer sample is modeled containing either SGMs (left), no additive (middle), or a hollow air pocket to approximate HGMs. Low density polyethylene (LDPE) is used as the polymer in place of poly(cis-cyclooctene) since it was already integrated into the software and has similar properties. Additive size is 50, 100, or 300 μm, with the number of particles inversely proportional to size. The polymer properties and ultrasound parameters used in the simulation are given in the FIGS. 30 and 31. Larger sized particles in the composites result in a more localized heating effect. In addition, air pockets result in higher temperatures compared with SGMs or no additive. It is noted that the polymer is surrounded by tissue in the simulation, whereas in ultrasound testing the polymer sits on a thin layer of water in the dish and is otherwise exposed to air in order to monitor the temperature with the thermal camera. Tissue is contemplated to limit the maximum temperatures reached in the polymer due to its higher thermal conductivity as compared with air. Thus, simulation 1 is most relevant to conditions in vivo, where the polymer is surrounded by tissue. Importantly, the highest temperatures are constrained to the polymer interior, limiting the exposure of tissue to thermal damage. (FIGS. 30A-30C and FIGS. 31A-31C)

A second simulation (Simulation 2) is performed where instead air is added in place of tissue to more closely model the experimental setup. The polymer reaches significantly higher temperatures in some samples. Different behaviors are observed as compared with certain other cases. For the bubble case, temperature maps show uniform distribution and maximum heating with 50 μm bubbles. 100 and 300 μm cases have localized heating within the lower part of the matrix, and the max temperatures are smaller. For the SGM case, the uniform temperature distribution and the max temperature occur with 100 μm SGMs. For 50 μm SGMs, very small heating is contemplated. For 300 μm, very localized heating occurs at the bottom edge. In summary, the results roughly reflect observations during the experiments, particularly greatest heating with smallest HGMs, iM30k (15.3 μm), followed by S35 (40 μm), and K25 (55 μm). When SGMs are used, the highest temperature is observed with medium-sized glass beads (Avg. size 500 μm) instead of smaller or bigger glass beads. These results suggest that at least the trend of performance of various additives can be simulated, while more quantitative agreement may be achievable in future modeling.

### REFERENCES CITED ABOVE

1. Delaey, J.; Dubruel, P.; Van Vlierberghe, S., Shape-Memory Polymers for Biomedical Applications. Advanced Functional Materials 2020, 30 (44), 1909047.
2. Han, J.; Fei, G.; Li, G.; Xia, H., High Intensity Focused Ultrasound Triggered Shape Memory and Drug Release from Biodegradable Polyurethane. Macromolecular Chemistry and Physics 2013, 214 (11), 1195-1203.
3. Lendlein, A.; Langer, R., Biodegradable, Elastic Shape-Memory Polymers for Potential Biomedical Applications. Science 2002, 296 (5573), 1673-1676.
4. Yeazel, T. R.; Becker, M. L., Advancing Toward 3D Printing of Bioresorbable Shape Memory Polymer Stents. Biomacromolecules 2020, 21 (10), 3957-3965.
5. Maloney, E.; Hwang, J. H., Emerging HIFU applications in cancer therapy. International Journal of Hyperthermia 2015, 31 (3), 302-309.
6. Izadifar, Z.; Izadifar, Z.; Chapman, D.; Babyn, P., An Introduction to High Intensity Focused Ultrasound: Systematic Review on Principles, Devices, and Clinical Applications. Journal of Clinical Medicine 2020, 9 (2).
7. Li, G.; Yan, Q.; Xia, H.; Zhao, Y., Therapeutic-Ultrasound-Triggered Shape Memory of a Melamine-Enhanced Poly(vinyl alcohol) Physical Hydrogel. ACS Applied Materials & Interfaces 2015, 7 (22), 12067-12073.
8. Li, G.; Fei, G.; Liu, B.; Xia, H.; Zhao, Y., Shape recovery characteristics for shape memory polymers subjected to high intensity focused ultrasound. RSC Advances 2014, 4 (62), 32701-32709.
9. Bhargava, A.; Peng, K.; Stieg, J.; Mirzaeifar, R.; Shahab, S., Focused ultrasound actuation of shape memory polymers; acoustic-thermoelastic modeling and testing. RSC Advances 2017, 7 (72), 45452-45469.
10. Calderon, N.; Morris, M. C., Melting temperature of trans-polyoctenamer. Journal of Polymer Science Part A-2: Polymer Physics 1967, 5 (6), 1283-1292.
11. Liu, C.; Chun, S. B.; Mather, P. T.; Zheng, L.; Haley, E. H.; Coughlin, E. B., Chemically Cross-Linked Polycyclooctene: Synthesis, Characterization, and Shape Memory Behavior. Macromolecules 2002, 35 (27), 9868-9874.
12. Athanassiadis, A. G.; Ma, Z.; Moreno-Gomez, N.; Melde, K.; Choi, E.; Goyal, R.; Fischer, P., Ultrasound-Responsive Systems as Components for Smart Materials. Chemical Reviews 2021.
13. You, S.; Chen, M.-W.; Dlott, D. D.; Suslick, K. S., Ultrasonic hammer produces hot spots in solids. Nature Communications 2015, 6 (1), 6581.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Certain molecules disclosed herein may contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every composition, material, formulation, device, system, combination of components, or method described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1-73. (canceled)

74. A composite material comprising:
one or more shape memory polymers; and
a first additive provided in the one or more shape memory polymers; wherein:
(a) the first additive comprises a plurality of inorganic particles, (b) the first additive increases an ultrasound attenuation coefficient of the composite material compared to that of the same one or more shape memory polymers free of said first additive; and/or (c) the first additive comprises a plurality of hollow particles;
the composite material is characterized by a composite transition temperature (T_{cm,trans});
the first additive is provided at least at one or more portions of the composite material; and
the composite material or the one or more portions thereof undergo a shape change from a temporary shape to a permanent shape when the composite material or said one or more portions thereof are heated to within 35° C. of T_{cm,trans} or a temperature approximately equal to or greater than T_{cm,trans}.

75. The composite material of claim 74, wherein the first additive is provided throughout the internal volume of the one or more shape memory polymers.

76. The composite material of claim 74, wherein the one or more portions of the composite material having the first additive are heated when said one or more portions of the composite material are exposed to ultrasound.

77. The composite material of claim 76, wherein the first additive absorbs the ultrasound; and wherein the first additive is heated by its absorption of ultrasound and/or wherein heat is generated by friction between the first additive and the one or more shape memory polymers when the first additive absorbs the ultrasound.

78. The composite material of claim 74, wherein the composite material undergoes the shape change only at the one or more portions thereof having the first additive exposed to the ultrasound.

79. The composite material of claim 74, wherein the first additive increases the ultrasound attenuation coefficient of the composite material or of the one or more portions thereof having the first additive by at least 100% compared to that of the same one or more shape memory polymers free of said first additive.

80. The composite material of claim 74, wherein the composite material or at least the one or more portions thereof are characterized by an ultrasound attenuation coefficient selected from the range of 0.05 dB/mm to 10 dB/mm.

81. The composite material of claim 74, wherein T_{cm,trans} is selected from the range of 25 to 100° C.

82. The composite material of claim 81, wherein T_{cm,trans} is the melt transition temperature (Tₘ) of the composite material.

83. The composite material of claim 74, wherein shape change is an expansion, a contraction, a twisting, an unraveling, a curling, an unfurling, an opening, a closing, a bending, an unbending, a folding, an unfolding, a straightening, a lengthening, a shortening, a redistribution or change in distribution of stress in the material, a redistribution or change in distribution of strain in the material, or any combination of these.

84. The composite material of claim 74, wherein the shape change occurs as a result of exposure of the composite material or the one or more portions thereof to ultrasound characterized by frequencies selected from the range of approximately 300 kHz to approximately 3 MHz and an energy intensity selected from the range of approximately 1 W/cm² to approximately 3 W/cm².

85. The composite material of claim 74, wherein the one or more portions having the first additive exhibit heating at a rate of 0.1° C./s to 5° C./s with exposure to ultrasound characterized by frequencies selected from the range of approximately 300 kHz to approximately 3 MHz and an energy intensity selected from the range of approximately 1 W/cm² to approximately 3 W/cm².

86. The composite material of claim 74, wherein the composite material is characterized by Young's modulus selected from the range of 1.0 MPa to 1000 MPa at NTP.

87. The composite material of claim 74, wherein the first additive comprises a plurality of inorganic particles.

88. The composite material of claim 74, wherein the first additive in the composite material is characterized by an ultrasound attenuation coefficient selected from the range of 0.05 dB/mm to 10 dB/mm.

89. The composite material of claim 74, wherein the first additive comprises a plurality of hollow particles.

90. The composite material of claim 74, wherein the first additive comprises a plurality of hollow glass beads, non-hollow glass beads, or any combination thereof.

91. The composite material of claim 74, wherein the first additive comprises a plurality of salt particles, a plurality of metal oxide particles, a plurality of metal particles, a plurality of organic particles, or any combination thereof.

92. The composite material of claim 74, wherein the first additive comprises a plurality of hollow glass microspheres and/or non-hollow glass microspheres characterized by a median diameter selected from the range of 15 to 1000 μm.

93. The composite material of claim 92, wherein the first additive comprises a plurality of hollow microspheres characterized by a median internal cavity diameter selected from the range of 1 to 100 μm and/or an average wall thickness selected from the range of 0.8 to 1.2 μm.

94. The composite material of claim 74, wherein a concentration of the first additive in the one or more shape memory polymers is selected from the range of 0.5 wt. % to 50 wt. % with respect to weight of the one or more polymers.

95. The composite material of claim 74, wherein the first additive is biologically inert and/or is substantially insoluble in a biological fluid under physiological conditions.

96. The composite material of claim 74, wherein the first additive is characterized by a density selected from the range of 0.01 to 22.5 g/cm³, a heat capacity selected from the range of 0.11 to 4.2 JC⁻¹g⁻¹, and a thermal conductivity selected from the range of 0.02 to 428.00 Wm⁻¹K⁻¹; and wherein the first additive comprises a plurality of particles having a median characteristic size selected from the range of 0.030 to 1000 μm.

97. The composite material of claim 74, wherein one or more additive-free crosslinked shape memory polymers equivalent to the one or more shape memory polymers of the composite material are characterized by a polymer-only transition temperature (T_{pol,trans}); and wherein T_{cm,trans} deviates from T_{pol,trans} by no more than 5° C. and/or 10%.

98. The composite material of claim 74, wherein the one or more shape memory polymers comprise crosslinking moieties derived from a crosslinking precursor selected from the group consisting of an organic peroxide having a 10-hour half-life temperature (HLT) at least 10° C. greater than a melt temperature (Tₘ) of the one or more shape memory polymers.

99. The composite material of claim 74, wherein the one or more shape memory polymers comprise crosslinking moieties derived from a crosslinking precursor selected from the group consisting of: a di(4-cyclooctenol) succinate, dicumyl peroxide (DCP), dibenzoyl peroxide (DBzP), di(tert-butyl) peroxide, and any combination thereof.

100. The composite material of claim 74, wherein shape memory polymer comprises poly(cyclooctene), polycaprolactone, poly(lactic acid), poly(lactic-co-glycolic acid), polyethylene, polypropylene, thermoplastic polyurethane (TPU), or any combination thereof.

101. A device comprising:
a composite material;
wherein the composite material comprises:
one or more shape memory polymers; and
a first additive provided in the one or more shape memory polymers; wherein:
(a) the first additive comprises a plurality of inorganic particles, (b) the first additive increases an ultrasound attenuation coefficient of the composite material compared to that of the same one or more shape memory polymers free of first additive, and/or (c) the first additive comprises a plurality of hollow particles;
the composite material is characterized by the composite transition temperature (T_{cm,trans}); and
the one or more portions of the composite material undergo the shape change from a temporary shape to a permanent shape when the one or more portions are heated to within 35° C. of T_{cm,trans} or a temperature approximately equal to or greater than T_{cm,trans}.

102. A method of using a composite material, the method comprising:
directing one or more focused ultrasound beams at one or more portions of the composite material;
thereby, heating the one or more portions to a temperature approximately equal to or greater than a composite transition temperature (T_{cm,trans}); and
thereby, causing the composite material to undergo a shape change at the one or more portions thereof;
wherein the composite material comprises:
one or more shape memory polymers; and
a first additive provided in the one or more shape memory polymers; wherein:
(a) the first additive comprises a plurality of inorganic particles, (b) the first additive increases an ultrasound attenuation coefficient of the composite material compared to that of the same one or more shape memory polymers free of first additive, and/or (c) the first additive comprises a plurality of hollow particles;
the composite material is characterized by the composite transition temperature (T_{cm,trans}); and
the one or more portions of the composite material undergo the shape change from a temporary shape to a permanent shape when the one or more portions are heated to within 35° C. of T_{cm,trans} or a temperature approximately equal to or greater than T_{cm,trans}.

103. A method of making a composite material, the method comprising:
polymerizing a monomer to form a first polymer;
crosslinking the first polymer in the presence of a crosslinking precursor and a first additive at a temperature approximately equal to or greater than a crosslinking temperature (T_{cm,perm}) to form the composite material having the crosslinked shape memory polymer and the first additive;
wherein the composite material comprises:
the shape memory polymer; and
the first additive provided in the shape memory polymer; wherein:
(a) the first additive comprises a plurality of inorganic particles, (b) the first additive increases an ultrasound attenuation coefficient of the composite material compared to that of the same one or more shape memory polymers free of first additive, and/or (c) the first additive comprises a plurality of hollow particles;
the composite material is characterized by the composite transition temperature (T_{cm,trans}); and
the one or more portions of the composite material undergo the shape change from a temporary shape to a permanent shape when the one or more portions are heated to within 35° C. of T_{cm,trans} or a temperature approximately equal to or greater than T_{cm,trans}.
